(19) United States
(12) Patent Application Publication
(10) Pub. No.: US 2024/0207532 A1
(43) Pub. Date: Jun. 27, 2024

(54) CAP FOR USE WITH VARIETY OF INJECTION PENS AND RELATED DEVICES AND METHODS

(71) Applicant: Bigfoot Biomedical, Inc., Milpitas, CA (US)

(72) Inventors: Michael J. Wakim, Milpitas, CA (US); Olga Smelyanets, Milpitas, CA (US); Christopher Kovalchick, Milpitas, CA (US); Rachael Price, Milpitas, CA (US); Aaron Townsend, Milpitas, CA (US); Peter Holst, Milpitas, CA (US); Liam Parrish, Milpitas, CA (US); Alex Martinez, Milpitas, CA (US); Alec Lindeman, Milpitas, CA (US); Samue Sayre, Milpitas, CA (US)

(21) Appl. No.: 18/398,051

(22) Filed: Dec. 27, 2023

Related U.S. Application Data
(60) Provisional application No. 63/477,307, filed on Dec. 27, 2022.

(51) Int. Cl. A61M 5/32

(52) U.S. Cl. CPC A61M 5/3202

(57) ABSTRACT

A cap for use with a variety of injection pens includes one or more adaptable elements. The one or more adaptable elements may be configured to removably couple the pen cap to a plurality of different geometries of a plurality of different injection pens.

### TECHNICAL FIELD

This disclosure relates, generally, to caps for drug injection pens and medication therapy management.

BACKGROUND

Injection pens for dosing drugs (also referred to as a “drug dosing pen,” “dosing pen,” “drug injection pen,” or “medicine injection pen”) are used to deliver various medications and for medication-based therapies. For example, some therapies may include growth hormones, insulin, fertility medication, Homozygous Familial Hypercholesterolemia (HoFH) treatment, without limitation.

### BRIEF SUMMARY

The various embodiments described below provide benefits and/or solve one or more of the foregoing or other problems in the art with devices and methods for a universal pen cap for medicinal injection pens. Embodiments include a pen cap for use with injection pens where the pen cap comprises one or more adaptable elements. The adaptable elements configured to removably couple the pen cap to a plurality of different geometries of a plurality of different injection pens.

Some embodiments include a pen cap for interfacing with injection pens where the pen cap includes means for removably coupling the pen cap to a plurality of different geometric shapes of a plurality of different injection pens.

Additional embodiments include a pen cap for interfacing with injection pens where the pen cap includes one or more adaptable elements. The adaptable elements configured to removably couple the pen cap to a plurality of different geometries of a plurality of different injection pens. Moreover, the pen cap further includes an electromechanical actuator. The electromechanical actuator coupled to the one or more adaptable elements and configured to actuate the one or more adaptable elements to adapt the one or more adaptable elements to a given geometry of a given injection pen.

Further embodiments include a pen cap for interfacing with injection pens where the pen cap comprises means for removably coupling the pen cap to a plurality of different geometric shapes of a plurality of different injection pens. The pen cap includes an electromechanical actuator that is operably coupled to the means and is configured to at least partially effectuate operation of the means.

Still further embodiments include a method of actuating an electromechanical pen cap including detecting a pen cap event using one or more sensors and actuating a clasping mechanism responsive to the event.

Still further embodiments include a pen cap for interfacing with medical injection pens where the pen cap includes one or more adjustable floor elements. The adjustable floor elements configured to adjust the distance an injection pen inserts into the pen cap and, where the one or more adjustable floor elements includes a floor block element defining a cavity configured to accommodate a geometry of an injection pen.

### BRIEF DESCRIPTION OF THE DRAWINGS

Various embodiments will be described and explained with additional specificity and detail through the use of the accompanying drawings in which:

FIG. 1A shows a side view of an injection pen inserted into a conventional pen cap;

FIG. 1B shows a side view of an injection pen separate from a conventional pen cap;

FIG. 2 shows multiple injection pens with correlating conventional pen caps;

FIG. 3A shows a side view of a universal pen cap in a disengaged configuration according to one or more embodiments of the present disclosure;

FIG. 3B shows a side view of the universal pen cap shown in FIG. 3A in an engaged configuration;

FIG. 4A shows a side view of a universal pen cap according to one or more additional embodiments of the present disclosure;

FIG. 4B shows a perspective view of the universal pen cap of FIG. 4A with a housing thereon;

FIG. 5A shows an enlarged perspective view of a cam element of the universal pen cap of FIG. 4.

FIG. 5B shows an enlarged perspective view of a cam assembly of the universal pen cap of FIG. 4 in a first orientation;

FIG. 5C shows an enlarged perspective view of the cam assembly of the universal pen cap of FIG. 4A in a second orientation;

FIG. 6A shows a side view of a universal pen cap in a disengaged position according to one or more embodiments of the present disclosure;

FIG. 6B shows a cross-sectional view of the universal pen cap of FIG. 6A in the disengaged position;

FIG. 6C shows a cross-sectional view of the universal pen cap of FIGS. 6A and 6B in an engaged position;

FIG. 7A shows a side view of a universal pen cap according to one or more embodiments of the present disclosure disengaged from an injection pen;

FIG. 7B shows a cross-sectional side view of the universal pen cap of FIG. 7A;

FIG. 7C shows a front view of the universal pen cap of FIGS. 7A viewed down a center longitudinal axis of the universal pen cap;

FIG. 8 shows a box diagram illustrating an operable connection between the universal pen cap of FIG. 7A and an electromechanical actuator according to one or more embodiments of the present disclosure;

FIG. 9A shows a perspective view of a universal pen cap according to one or more embodiments of the present disclosure;

FIG. 9B shows a cross-sectional side view of the universal pen cap of FIG. 9A extended over an injection pen;

FIG. 10A shows a side view of a universal pen cap according to one or more embodiments of the present disclosure;

FIG. 10B shows a cross-sectional side view of the universal pen cap of FIG. 10A according to one or more embodiments;

FIG. 11A shows a perspective view of a universal pen cap and a plurality of injection pens according to one or more embodiments of the present disclosure;

FIG. 11B shows a cross sectional side view of the universal pen cap and the plurality of injection pens of FIG. 11A;

FIG. 11C shows a cross-sectional side view of the universal pen cap of FIG. 11A with an injection pen inserted therein;

FIG. 12 shows a perspective view of a universal pen cap according to one or more embodiments of the present disclosure;

FIG. 13A shows a perspective view of a universal pen cap according to one or more embodiments;

FIG. 13B shows a front view of the universal pen cap of FIG. 13A in a disengaged configuration;

FIG. 13C shows a front view of the universal pen cap of FIG. 13A in an engaged configuration;

FIG. 14 shows a box diagram illustrating an operable connection between the universal pen cap of FIG. 13A and an electromechanical actuator;

FIG. 15A shows a perspective view of a universal pen cap in a disengaged configuration according to one or more embodiments of this disclosure;

FIG. 15B shows a perspective view of the universal pen cap of FIG. 15A in an engaged configuration according to one or more embodiments;

FIG. 15C shows a cross-sectional side view of the universal pen cap of FIG. 15A engaged with an injection pen according to one or more embodiments;

FIG. 16 shows a box diagram illustrating an operable connection between the universal pen cap of FIG. 15A and an electromechanical actuator;

FIG. 17 shows a cross-sectional side view of a universal pen cap, according to one or more embodiments of the present disclosure;

FIG. 18A shows a perspective view of a universal pen cap according to one or more embodiments of this disclosure;

FIG. 18B shows a front view of the universal pen cap of FIG. 18A looking down a longitudinal axis of the universal pen cap;

FIG. 19A shows a perspective view of a universal pen cap in an engaged configuration according to one or more embodiments;

FIG. 19B shows a perspective view of the universal pen cap of FIG. 19A in a disengaged configuration;

FIG. 20A shows a perspective view of a universal pen cap in an engaged configuration according to one or more embodiments of the present disclosure;

FIG. 20B is a perspective view of the universal pen cap of FIG. 20A in a disengaged configuration;

FIG. 21A is a perspective view of a universal pen cap according to one or more embodiments of this disclosure;

FIG. 21B is a side view the universal pen cap of FIG. 21A having an injection pen inserted therein;

FIG. 21C is a side view of the universal pen cap of FIGS. 21A and 22B in a disengaged condition;

FIG. 21D is a side view of the universal pen cap of FIGS. 21A-21C in an engaged condition;

FIG. 21E is a perspective view of the universal pen cap of FIGS. 21A-21D in the engaged condition;

FIG. 22A shows a side view of a pen clicking mechanism that may be used with the universal pen cap of FIGS. 21A-21B;

FIG. 22B shows a perspective view of the pen clicking mechanism of FIG. 22A in a disengaged position;

FIG. 22C shows a perspective view of the pen clicking mechanism of FIG. 22A in an engaged position;

FIG. 23A shows a perspective view of a universal pen cap having an electromechanical actuator, according to one or more embodiments of the present disclosure;

FIG. 23B shows a cross-sectional perspective view of the universal pen cap of FIG. 23A;

FIG. 24 shows a semi-transparent side view of a floor block element according to one or more embodiments of the present disclosure;

FIG. 25A shows a semi-transparent side view of a universal pen cap having adjustable floor elements according to one or more embodiments of the present disclosure;

FIG. 25B shows an exploded side view of the universal pen cap having adjustable floor elements of FIG. 25A;

FIG. 26A shows a floor block element partially inserted into a universal pen cap according to one or more embodiments;

FIG. 26B shows the floor block element of FIG. 26A having an injection pen inserted therein;

FIG. 27A shows a cross-sectional side view of a universal pen cap including an adjustable floor system in a first position according to one or more embodiments of the present disclosure;

FIG. 27B shows a cross-sectional side view of the universal pen cap of FIG. 27A including the adjustable floor system in a second position;

FIG. 28A shows a cross-sectional side view of a universal pen cap including an adjustable floor system in a first position according to one or more embodiments of the present disclosure;

FIG. 28B shows a cross-sectional side view of the universal pen cap of FIG. 28A including the adjustable floor system in a second position;

FIG. 29 is a cross-sectional side view of a universal pen cap including an adjustable floor system according to one or more embodiments of the present disclosure;

FIG. 30 shows a flow diagram of a method of operation for a universal pen cap according to one or more embodiments of the present disclosure;

FIG. 31 a block diagram of an exemplary system that includes a universal pen cap, one or more sensors, and an electromechanical actuator, according to one or more examples;

FIG. 32 shows a flow diagram of a method for actuating one or more adaptable elements of a universal pen cap;

FIG. 33 shows a flow diagram of a method for actuating one or more adaptable elements of a universal pen cap; and

FIG. 34 illustrates a flow diagram of an exemplary computing device in accordance with one or more embodiments.

FIG. 35 illustrates a schematic diagram of an exemplary computing device in accordance with one or more embodiments.

FIG. 36A illustrates a side explosion view of a portion of a universal pen cap, according to another embodiment.

FIG. 36B illustrates a side explosion view of a clamping mechanism of the universal pen cap shown in FIG. 36A.

FIG. 36C illustrates a side explosion view of a clicker mechanism of the universal pen cap shown in FIG. 36A.

FIG. 37A illustrates a front perspective view of a pen clamp, according to one embodiment.

FIG. 37B illustrates a front perspective view of two pen clamps clamping a dosing pen, according to one embodiment.

FIG. 38 illustrates a side transparent view of a portion of a universal pen cap, according to one embodiment.

FIG. 39A-1 illustrates a side view of a clicker mechanism in State O, according to one embodiment.

FIG. 39A-2 illustrates a schematic view of the clicker mechanism in State O, according to one embodiment.

FIG. 39B-1 illustrates a side view of a clicker mechanism in State 1, according to one embodiment.

FIG. 39B-2 illustrates a schematic view of the clicker mechanism in State 1, according to one embodiment.

FIG. 39C-1 illustrates a side view of a clicker mechanism in State 2, according to one embodiment.

FIG. 39C-2 illustrates a schematic view of the clicker mechanism in State 2, according to one embodiment.

FIG. 39D-1 illustrates a side view of a clicker mechanism in State 2.5, according to one embodiment.

FIG. 39D-2 illustrates a schematic view of the clicker mechanism in State 2.5, according to one embodiment.

FIG. 39E-1 illustrates a side view of a clicker mechanism in State 3, according to one embodiment.

FIG. 39E-2 illustrates a schematic view of the clicker mechanism in State 3, according to one embodiment.

FIG. 39F-1 illustrates a side view of a clicker mechanism in State 4, according to one embodiment.

FIG. 39F-2 illustrates a schematic view of the clicker mechanism in State 4, according to one embodiment.

FIG. 40A illustrates a front view of a second clicker body of a clicker mechanism viewed down a center longitudinal axis of the universal pen cap, according to one embodiment.

FIG. 40B illustrates a front view of a first clicker body of a clicker mechanism interacting with the second clicker body shown in FIG. 40A viewed down a center longitudinal axis of the universal pen cap, according to one embodiment.

FIG. 41 illustrates an internal side view of a portion of a universal pen cap with an ingress wall, according to one embodiment.

FIG. 42 illustrates a top perspective view of a portion of a universal pen cap, according to one embodiment.

FIG. 43 illustrates a side view of a frictional clasp used for clamping a dosing pen, according to one embodiment.

FIG. 44A illustrates a front view of the frictional clasp shown in FIG. 43 clamping a dosing pen in a first orientation viewed down a center longitudinal axis of the universal pen cap, according to one embodiment.

FIG. 44B illustrates a front view of the frictional clasp shown in FIG. 43 clamping a dosing pen in a second orientation viewed down a center longitudinal axis of the universal pen cap, according to one embodiment.

FIG. 44C illustrates a front view of the frictional clasp shown in FIG. 43 clamping a dosing pen in a third orientation viewed down a center longitudinal axis of the universal pen cap, according to one embodiment.

### DETAILED DESCRIPTION

The illustrations presented herein are not actual views of any injection delivery and data collection system, or any component thereof, but are merely idealized representations, which are employed to describe the present invention.

Diabetes mellitus is a chronic metabolic disorder caused by the inability of a person's pancreas to produce sufficient amounts of the hormone insulin such that the person's metabolism is unable to provide for the proper absorption of sugar and starch. The inability to absorb those carbohydrates sometimes leads to hyperglycemia, i.e., the presence of an excessive amount of glucose within the blood plasma. Hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities.

Often, a permanent therapy is necessary to maintain a proper blood glucose (amount of glucose in a person's bloodstream or a glucose value (also referred to herein as an “estimated glucose value”) representative of the same, such as a blood glucose level taken by a blood glucose meter or a glucose level generated by a glucose monitor) within normal limits Maintaining a proper glucose level is conventionally achieved by regularly supplying insulin to a person with diabetes (PWD). Maintaining a proper glucose value may create a significant cognitive burden for a PWD (or a caregiver) and affect many aspects of the PWD's life. For example, the cognitive burden on a PWD may be attributed to, among other things, tracking meals and constant check-ins and minor course corrections of glucose values. The adjustments of glucose values by a PWD may include taking insulin, tracking insulin dosing and glucose, deciding how much insulin to take, how often to take it, where to inject the insulin, and how to time insulin doses in relation to meals and/or glucose fluctuations.

The following example of a typical daily routine for a PWD further illustrates the significant cognitive burden of a PWD. In the morning, the first thoughts/actions by a PWD are often related to their glucose, such as, what is their glucose value? How was their glucose value overnight? And how are they currently feeling? Upon checking their glucose values (e.g., using a blood glucose meter or monitor), a PWD may then consider what actions to take, such as adjusting their morning activities, changing when or what to eat for breakfast, or determining to take rapid-acting (RA) insulin and deciding where to inject the rapid-acting (RA) insulin. Before they even eat breakfast (or any meal), a PWD considers the amount of food and types of food they plan to eat, perhaps modifying their RA insulin dose based on the carbohydrate content of the food they choose to eat. Before they administer RA insulin, the PWD will try to remember when they took their last dose of insulin, what happened the last time they ate a particular meal and how they felt.

Before leaving the house, a PWD considers, among other things, whether they have enough supplies for glucose monitoring or insulin dosing. This may include batteries, charged devices, backup supplies, glucose testing supplies, and insulin supplies to treat for high glucose values. Additionally, a PWD needs to consider any physical activities (e.g., walking kids to school, going to the gym, riding a bike) that will affect their glucose because exercise may cause their blood glucose to go lower than expected. Even before driving a vehicle, a PWD checks their glucose to determine if it is at a safe level for driving.

As lunchtime approaches, a PWD considers their glucose prior to eating lunch, such as what time they may expect to eat, what they expect to eat throughout the day. As such, a PWD tallies up the carbohydrates and adjusts insulin doses in their head. A PWD also considers what insulin doses were recently taken and whether those doses may still be working to lower blood glucose. This is all done in parallel with whatever they are doing in their busy day, and so the PWD often forgets or fails to fully consider all of the factors described above.

Throughout the day, a PWD often checks glucose levels, especially on days when their activities vary from a typical day. This constant thinking, checking, planning may be exhausting, especially when each check requires decisions, math, and possible behavior changes. Additionally, during the day, a PWD may check inventory on supplies, speak with a health care provider (HCP), refill prescriptions, and contact their health insurance to discuss their therapy and/or supplies.

In the evening, a PWD may have to take a daily insulin dose of long-acting (LA) insulin. Additionally, the PWD may determine if their glucose is holding steady before they fall asleep. If they use an infusion pump, they have to check if their insulin pump is low on insulin and whether they need to refill it before sleep. If they have a continuous glucose monitor, they have to check and see if it is working. Even then, based on what they ate for dinner, the nighttime insulin might not keep their glucose steady. Glucose levels in the night may interfere with sleep as well as add anxiety that could disrupt sleep.

Accordingly, managing diabetes requires significant attention to detail throughout the day. Even with careful planning and self-monitoring, a PWD may skip doses, double dose, or dose the wrong amount and/or type of insulin. Insufficient insulin may result in hyperglycemia, and too much insulin may result in hypoglycemia, which may result in clumsiness, trouble talking, confusion, loss of consciousness, seizures, or death.

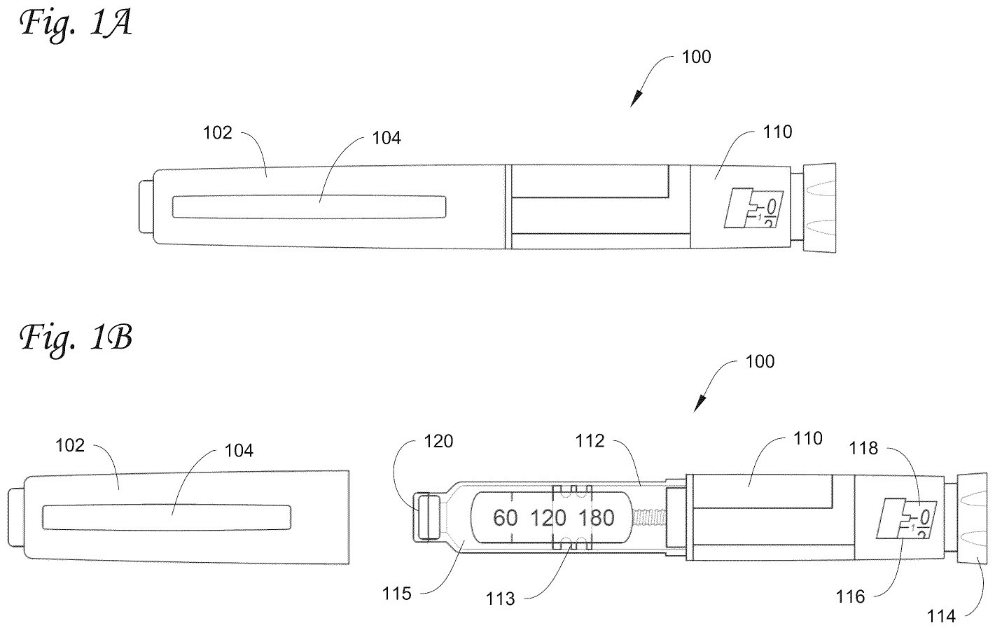

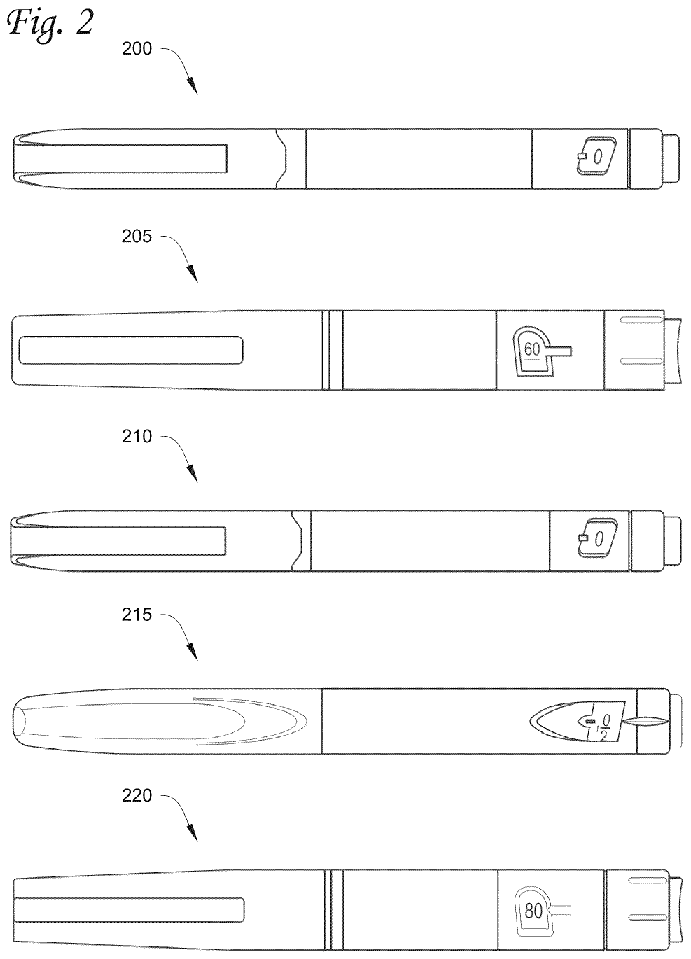

One of the most common methods of administering insulin doses involves using a drug dosing pen (a drug dosing pen utilized to dose insulin may be referred to herein as an “insulin pen”). Because a PWD may need to take insulin at a moment's notice, a PWD is often forced to carry around one or more drug dosing pens (e.g., See FIG. 1A and FIG. 1B). Dosing pens often include a cap to improve portability and to prevent loss of the medicinal drug contained inside as well as to protect against accidental punctures or injections. In addition to the different types of drugs/medicines (e.g., different types of insulin) that can be contained in a dosing pen, the geometry of different dosing pens may vary widely. As shown in FIG. 1A and FIG. 1B, a dosing pen 100 is provided. The dosing pen 100 includes a pen cap 102 with a cap clip 104, and a dosing pen body 110. The dosing pen body 110 includes a cartridge holder 112 for housing a removable pen cartridge 115 and a plunger 113, a dose knob 114, and a dose window 116 with a dose indicator 118. The dosing pen body 110 also includes a rubber seal 120 disposed at an end opposite the dose knob 114 and adjacent to where the removable pen cartridge 115 is disposed. For example, dosing pens may differ in pen barrel diameter, pen barrel length, label placement, nib geometry, nib placement, and other features. Moreover, different brands of insulin pens may vary widely in their shape characteristics, even for insulin pens having the same type of insulin dose (e.g., see FIG. 2). In particular, FIG. 2 shows a Toujeo® Max SoloStar® insulin pen 200, a Tresiba® FlexTouch® U-200 insulin pen 205, a Toujeo® SoloStar® insulin pen 210, a Lantus® SoloStar® U-100 insulin pen 215, and a Tresiba® FlexTouch® U-100 insulin pen 220. Furthermore, over the course of therapy, a user may switch between multiple insulin brands based on insurance, therapy, preference, without limitation. Typically, each dosing pen has its own pen cap specifically designed to work with the dosing pen's geometry. Accordingly, a PWD may have multiple dosing pens, where the caps for each of the dosing pens may differ widely in shape and size.

A challenge for many PWDs is diabetes fatigue syndrome (DFS). DFS is a multifactorial syndrome that is commonly accompanied by fatigue or increased fatigability in people that have diabetes. The plethora of different insulin pens (including the potentially widely variable geometry between each pen) may increase learning obstacles for a user as well as increasing the user's cognitive burden when managing their diabetes on top of the already overwhelming list of tasks a PWD has to worry about on a daily basis. These problems are exacerbated further for users that have DFS or other cognitive impairing conditions. Because of the highly specific dosing requirements for using insulin to treat diabetes, these learning and cognitive burdens imposed by the varying physical characteristics of insulin pens may create a greater risk of dosing mistakes or lead to the inability to apply a dose, which may, as mentioned above, result in clumsiness, trouble talking, confusion, loss of consciousness, seizures, or even death.

Embodiments of the present disclosure include a universal pen cap that is adapted to receive a plurality medicinal injection pens (e.g., insulin injection pens) having varying geometries, and in particular, varying geometries at the tip ends thereof. For example, a universal pen cap for use with injection pens includes one or more adaptable elements arranged to removably couple the universal pen cap to a plurality of different geometries of a plurality of different injection pens. In various embodiments, the universal pen cap includes a mechanical actuator configured to modify one or more adaptable elements. In various embodiments the universal pen cap includes an electromechanical actuator that coupled to the one or more adaptable elements for modification thereof. The electromechanical actuator is adapted to actuate the one or more adaptable elements to adjust the one or more adaptable elements to a select geometry to match, such as substantially match, a given geometry of a given injection pen, and in particular, a given geometry of a tip end thereof. In various embodiments the universal pen cap includes one or more sensors for sensing various pen cap events, such as disengagement and engagement of the universal pen cap from and to the pen. The one or more sensors configured to enable the universal pen cap to automatically configure the universal pen cap responsive to a sensed action by a user. In various embodiments the universal pen cap includes adjustable floor elements that adjust the distance an injection pen inserts into the universal pen cap. The adjustable floor elements configured to adjust mechanically by a user or electromechanically by an electromechanical actuator operably coupled to the adjustable floor elements. In various embodiments, universal pen cap includes one or more batteries for powering various elements included in the universal pen cap (e.g., an electromechanical actuator, without limitation).

Furthermore, in various embodiments, the universal pen cap of an injection pen (e.g., an insulin pen) is configured for dose data capture. In various embodiments, the universal pen cap includes a display screen for displaying one or more of an estimated glucose value (EGV), units for the EGV, a trend indicator for the EGV, a recommended dosage, an identification of the type of insulin, a recommended site injection, a time and amount of a previous dosage, and/or an insulin on board value to remind a user about their most recent dosage. In various embodiments, the universal pen cap includes buttons for receiving meals information, insulin dose information, responses to recommendations, and the like, from a user. In various embodiments, the buttons are chosen from physical buttons, capacitive or resistive touch buttons, buttons on the display, or a combination/sub-combination thereof. In various embodiments, the display screen includes a touch screen configured to include one or more capacitive touch buttons in a user interface thereof. In various embodiments, a universal pen cap for dose-capture includes one or more indicator lights, configured to light up to indicate that it is transferring data, light up to indicate that the user's attention is needed, and/or light up to indicate whether a dose capture functionality is or is not working.

Embodiments of the universal pen cap of the present disclosure may be advantageous over conventional injection pen caps typically used for medicinal/drug injection pens. For example, the conventional approach of having a pen cap being designed to fit on only one specific pen geometry can impose cognitive burdens on PWD's trying to manage a plurality of different injection pens on top of the already cognitively demanding task inherent in managing diabetes, especially PWDs with DFS. In contrast, the universal pen cap of the present disclosure is adapted to be used with many types of injection pens having varying geometries, thus allowing a PWD to use a disclosed universal pen cap with one or more of multiple types of pens resulting in consistency, predictability, and reusability for a PWD, thereby reducing cognitive burdens on the PWD, particularly in day-to-day insulin management. This reduction in cognitive burden may lead to less stress, enable faster and easier application of medicinal doses, and reduce the likelihood of failure to apply a necessary medicinal dose. Additionally, the inclusion of sensors and electromechanical actuators in various embodiments may enable automated insertion and removal of an injection pen and therefore provide additional advantages including greater usability, particularly for those PWDs who may have minor to sever physical impairments that may cause them to struggle removing a conventional pen cap, which is typically pressure fitted to the injection pen. For example, PWD's with DFS may struggle to remove conventional pen caps, as their physical condition may not allow them to produce enough grip strength to grasp and pull the pen cap from the injection pen. In contrast, various embodiments herein may allow for removal of a universal pen cap that requires relatively little physical exertion by a user.

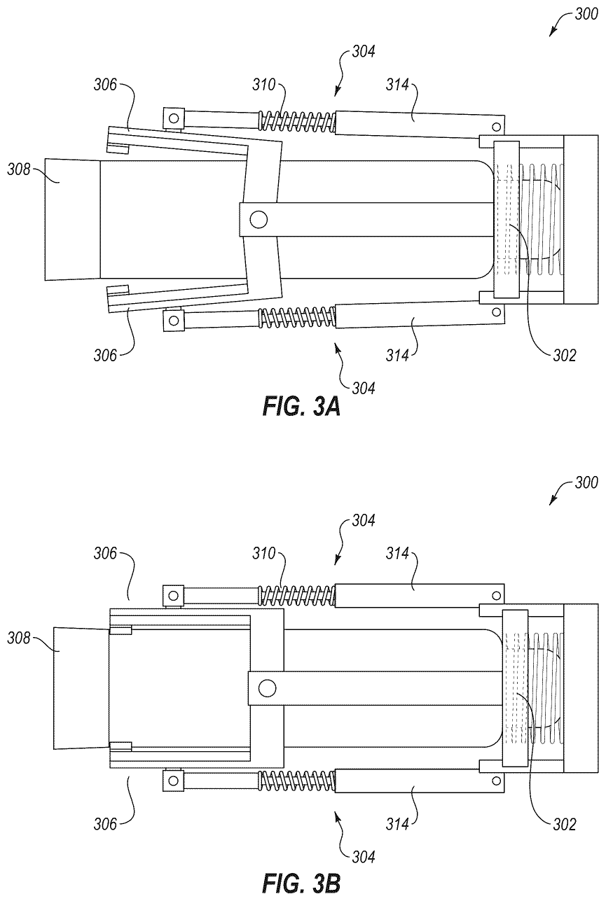

FIG. 3A is a side view of a universal pen cap 300 in a disengaged configuration according to one or more embodiments of the present disclosure. FIG. 3B is a side view of universal pen cap 300 of FIG. 3A in an engaged configuration. Referring to FIG. 3A and FIG. 3B together, in various embodiments, the universal pen cap 300 includes at least one compression actuator 302, actuating arms 304, and engagement members 306 that are operably coupled to compression actuator 302. In various embodiments, the at least one compression actuator 302 is adapted to cause the engagement members 306 to swing radially inward toward a center longitudinal axis of the universal pen cap 300 and engage (e.g., contact) the injection pen 308 in response to displacement of one or more portions of the at least one compression actuator 302, such as by an injection pen 308 being inserted into the universal pen cap 300 and pressed against at least one compression actuator 302.

In various embodiments, the actuating arms 304 are rotatably coupled to the engagement members 306 at a longitudinal end of the each of the actuating arms 304. For example, the actuating arms 304 in various embodiments are coupled to the engagement members 306 via a hinge connection. The actuating arms 304 each include an arm 314 and a biasing element 310 (e.g., a spring, without limitation). In various embodiments, the biasing element 310 is oriented along a longitudinal axis of the arm 314 and is configured to compress responsive to the actuation of the compression actuator 302 thereby causing the actuating arm 304 to bias the engagement members 306 radially inward and' at least partially cause the engagement members 306 to engage the injection pen 308. Accordingly, when the universal pen cap 300 is in a disengaged position (e.g., as shown in FIG. 3A), an injection pen (e.g., injection pen 308) may be inserted into the universal pen cap 300 which, upon substantial insertion displace at least part of the compression actuator 302, which in turn causes the biasing elements 310 of the actuating arms 304 to compress and also cause the engagement members 306 to pivot and rotate radially inward toward a center longitudinal axis of the universal pen cap 300 and engage the injection pen.

In various embodiments, the compression actuator 302 includes an end portion, a contact portion, a biasing element, and one or more connection arms. The end portion is adapted to receive the engagement portion. The contact portion is adapted to be contacted by the injection pen 308, such as at an end tip thereof and move axially relative to the end portion. The one or more connection arms extend from the contact portion in an axial direction opposite the end portion. The biasing element is positioned axially between the end portion and the contact portion and is adapted to bias the contact portion axially away from the end portion. In these embodiments, the engagement members 306 include a connection portion and an engagement portion. The connection portion rotatably connects to the one or more connection arms, such as at a distal end of the one or more connection arms via a joint. In these embodiments, the connection portion includes an annular shape, such as about half an annulus, with a connection to the one or more connection arms at an end thereof. In the embodiment illustrated, each end of the connection portion is rotatably coupled to a distal end of a connection arm, with each connection arm being on opposing sides of the universal pen cap 300, such as being circumferentially offset by 180 degrees or about 180 degrees. The engagement portion extends axially from the connection portion in a direction opposite the end portion. In the embodiment illustrated, the engagement portion is circumferentially offset from the ends thereof and from the joint formed with each connection arm by 90 degrees or about 90 degrees. With the engagement portion being clocked 90 degrees or about 90 degrees from the joints that the connection portion forms with the distal end of the connection arms, rotation of the engagement members 306 about the joints results in radial movement of a distal end of the engagement portion (distal relative to the connection portion) allowing the engagement members 306 to clamp down on the injection pen 308 inserted into the universal pen cap 300. In various embodiments, the engagement members 306 further include a clamping portion protruding radially inward from the distal end of the engagement portion. In embodiments, the clamping portion is adapted to directly contact the injection pen 308 and is adapted to clamp the injection pen 308 with sufficient force to secure the injection pen within the universal pen cap 300. In various embodiments, the universal pen cap 300 includes multiple actuating arms 304 with each actuating arm being circumferentially aligned with the engagement portion. In the embodiment illustrated, a corresponding actuating arm 304 the rotatable connection between the actuating arm 304 and the engagement portion is formed at a joint positioned between the distal and proximal ends of the engagement portion. In various embodiments, engagement between the injection pen 308 and contact portion causes the contact portion to compress biasing element and move axially towards the end portion, causing the connection arms to also move axially towards the end portion. Due to the connections of the connection portion to the connection arms and the engagement portion to the actuating arms 304, the axial movement of the connection arms causes the distal ends of the engagement portions to rotate radially inward and contact the injection pen 308 to secure the injection pen within the universal pen cap 300.

In various embodiments, when the universal pen cap 300 is in an engaged position (e.g., as shown in FIG. 3B) a subsequent actuation of an actuated compression actuator 302 causes the biasing elements 310 to pivot and rotate radially outward, thereby decreasing the inward radial force exerted by the actuating arm 304 on the engagement members 306 to release the injection pen 308 and enable removal of the injection pen 308.

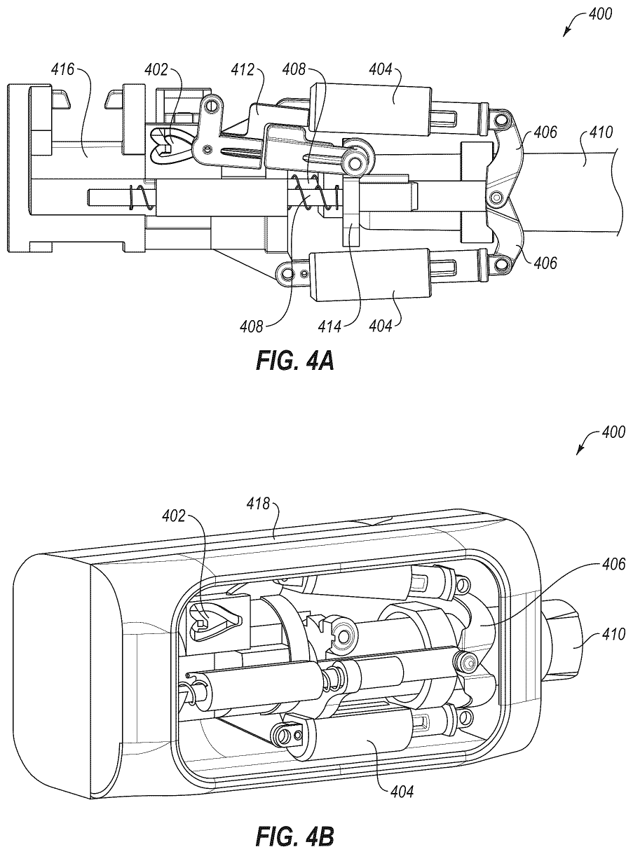

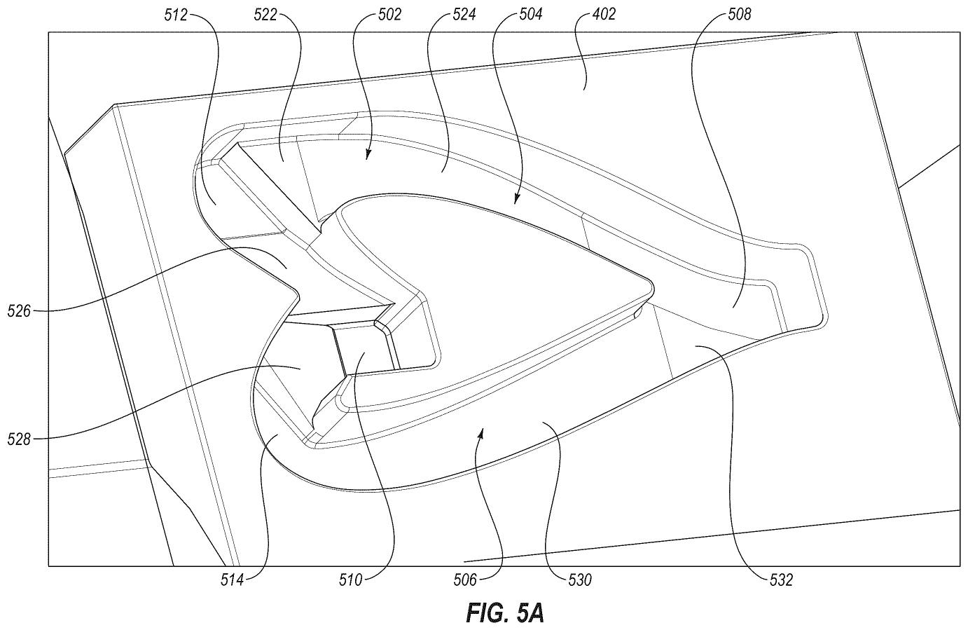

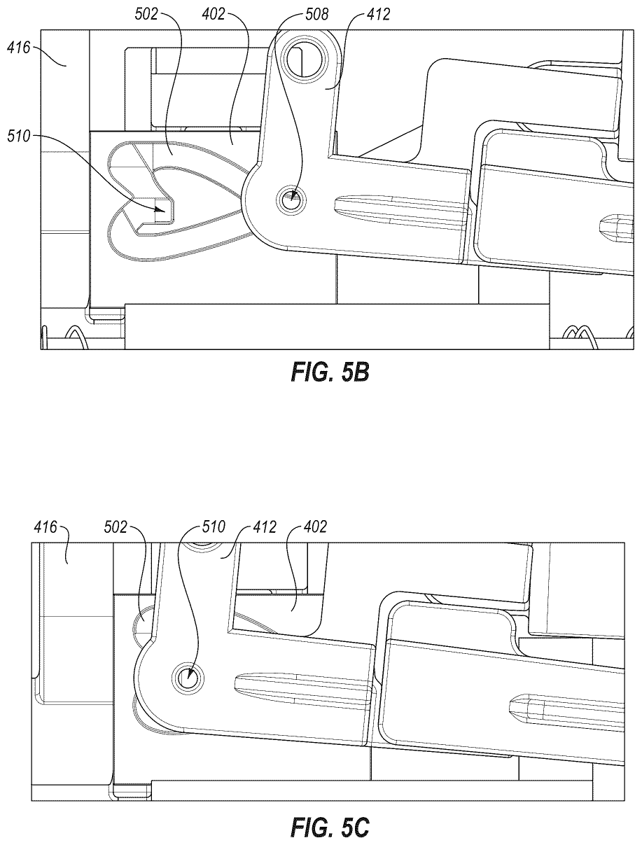

FIG. 4A is a side view of a universal pen cap 400 according to one or more embodiments. FIG. 4B shows a perspective view of the universal pen cap of FIG. 4A with a housing thereon. Referring to FIGS. 4A and 4B, in various embodiments, the universal pen cap 400 includes engagement members 406, actuating arms 404, a cam element 402, guide element 412, interface element 414, and body frame 416. In various embodiments, the engagement members 406 are rotatably coupled to the actuating arms 404, which in turn are mounted to the body frame 416. The engagement members 406 are configured to pivot relative to the actuating arms 404. The interface element 414 is sized and shaped to abut against a longitudinal end of an injection pen 410 when an injection pen 410 is inserted into the universal pen cap 400. The interface element 414 is coupled to the engagement members 406 and the guide element 412. Furthermore, the interface element 414 is coupled to the body frame 416 via one or more biasing elements 408, which biases the interface element 414 relative to body frame 416 in an axial direction (e.g., in a direction opposite to which an injection pen 410 is inserted into the universal pen cap 400). The engagement members 406 are configured to pivot relative to the interface element 414. The guide element 412 is rotatably coupled to the interface element 414 and is engaged with the cam element 402, which is described in further detail below. While a particular cam element 402 is illustrated in FIGS. 4A-5C, one skilled in the art will appreciate that other types of cam elements can also be used in actuating the universal pen cap 400.

In various embodiments, the engagement members 406 are sized and shaped to engage an injection pen 410. For instance, the engagement members 406 are be sized and shaped to define an opening there between sized to receive the injection pen 410. In various embodiments, the engagement members 406 have general jaw shapes and are configured to clamp the injection pen 410 in operation. In the embodiment illustrated, engagement between the injection pen 410 and the interface element 414 causes the interface element to translate towards the body frame 416 in the axial direction. The translation of the interface element 414 causes the joints between the interface element 414 and the engagement members 406 to also move in the axial direction towards the body frame 416. The axial movement of these joints causes the engagement members 406 to rotate relative to respective actuating arms 404 at the joint therebetween and causes the joints between the engagement members 406 and the actuating arms 404 to move radially inward. This radially inward movement causes the actuating arms 404 to rotate relative to the body frame 416. Due to the relative movements of the components, the engagement members 406 move radially inward and contact the injection pen 410 to secure the injection pen 410 within the universal pen cap 400, such as via clamping.

In various embodiments, the actuating arms 404 include one or more biasing members biasing the engagement members 406, to which the actuating arms 404 are coupled, in one or more directions away from the body frame 416. The actuating arms 404 are coupled to the engagement members 406 at radially outermost portions of the engagement members 406 such that when the engagement members 406 pivot relative to the actuating arms 404, the engagement members 406 rotate radially inward or outward.

As can be seen in FIG. 4B, in various embodiments, the universal pen cap 400 includes a housing 418 configured to encase the various elements of the universal pen cap 400 (e.g., engagement members 406, actuating arms 404, cam element 402, guide element 412, interface element 414, and body frame 416, without limitation). While the housing 418 is illustrated with regards to the one or more embodiments of FIGS. 4A and 4B, the housing 418 or a similar housing may be utilized with the various embodiments disclosed herein.

FIG. 5A is an enlarged perspective view of cam element 402, according to one or more embodiments. FIG. 5B is an enlarged view of cam element 402 and the guide element 412 when universal pen cap 400 is in a disengaged configuration, according to one or more embodiments. FIG. 5C is an enlarged view of cam element 402 and guide element 412 when universal pen cap 400 is in an engaged configuration.

Referring to FIGS. 4-5C together, in various embodiments, the cam element 402 includes a groove path 502 having a first pathway 504 and a second pathway 506 formed therein. The first pathway 504 includes a general hook shape that generally starts in a first direction and curves back to a second direction, opposite the first direction, and the second pathway 506 includes a general hook shape or a reverse hook shape. The first pathway 504 and the second pathway 506 are connected together at both longitudinal ends of the first pathway 504 and the second pathway 506 such that together, the first pathway 504 and the second pathway 506 form a general heart-shape groove where the second pathway 506 generally mirrors the first pathway 504. The cam element 402 is adapted to guide movement of a pin of the guide element 412 along the first pathway 504 from a first resting area 508 to a second resting area 510 and along the second pathway 506 from the second resting area 510 to the first resting area 508. In various embodiments, the groove path 502 is formed to prevent the pin from entering the second pathway 506 from the first resting area 508 and from entering the first pathway 504 from the second resting area 510.

The first pathway 504 includes the first resting area 508, a first sloped region 524, a first raised portion 522, a first recessed portion 512, and a second raised portion 526. The second pathway 506 includes the second resting area 510 connected to the second raised portion 526 of the first pathway 504, a second sloped region 528, a second recessed portion 514, and a third sloped region 530 connecting to the first resting area 508 of the first pathway.

The first sloped region 524 of the first pathway 504 extends from the first resting area 508 to the first raised portion 522. A depth of the first sloped region 524, relative to a top surface of the cam element 402, decreases from the first resting area 508 to the first raised portion 522. In various embodiments, the grade of the first sloped region 524 is such that a pin of the guide element 412 does not catch or stall thereon due to friction between the pin and a surface of the first sloped region 524. In various embodiments, the grade of the first sloped region 524 is variable. The first recessed portion 512 adjoins the first raised portion 522 and is formed at a depth lower than the first raised portion 522 relative to the top surface. The difference in depth formed between the first raised portion 522 and the first recessed portion 512 being adapted to prevent the pin of the guide element 412 from traveling from the first recessed portion 512 to the first raised portion 522. The first raised portion 522 terminates at an abrupt edge transitioning the first pathway 504 from the first raised portion 522 to the first recessed portion 512. For instance, the first recessed portion 512 includes a deeper depth relative to the first raised portion 522 such that when a pin of the guide element 412 travels along the first pathway 504, the pin ascends upward from the first resting area 508 to the first raised portion 522 along the first sloped region 524 and then falls into the first recessed portion 512. The second raised portion 526 slopes upward from the first recessed portion 512 toward the second pathway 506. The second raised portion 526 is formed with a variable slope. The second raised portion 526 abruptly end with an edge (e.g., a cliff edge) at the second resting area 510 of the second pathway 506 with the edge being formed at a depth higher than that of an end thereof adjacent to the first raised portion 522. The second resting area 510 is formed at a depth lower than the edge of the second raised portion 526. The difference in depth formed between the second resting area 510 and the second raised portion 526 being adapted to prevent the pin of the guide element 412 from traveling from the second resting area 510 to the second raised portion 526.

The second sloped region 528 of the second pathway 506 slopes upward from the second resting area 510 and abruptly end with an edge (e.g., a cliff edge) at the second recessed portion 514 of the second pathway 506 with the edge being formed at a depth higher than an end adjoining the second resting area 510 and at a depth higher than the second recessed portion 514. The difference in depth formed between the second recessed portion 514 and the edge of the second sloped region 528 being adapted to prevent the pin of the guide element 412 from traveling from the second recessed portion 514 to the second sloped region 528. In various embodiments, the end adjoining the second resting area 510 includes an edge with the end adjoining the second resting area 510 having less depth than the second resting area 510. This edge is formed to prevent the pin from leaving the second resting area 510 without a force being applied on the pin of the guide element 412. The third sloped region 530 of the second pathway 506 extends from the second recessed portion 514 toward the first resting area 508 and abruptly end with an edge (e.g., a cliff edge) at the first resting area 508 of the first pathway 504. A depth of the third sloped region 530, relative to the top surface of the cam element 402, decreases from the second recessed portion to the edge of the third sloped region 530. In various embodiments, the grade of the third sloped region 530 is such that a pin of the guide element 412 does not catch or stall thereon due to friction between the pin and a surface of the third sloped region 530. In various embodiments, the grade of the third sloped region 530 is variable. The difference in depth formed between the first resting area 508 and the edge of the third sloped region 530 being adapted to prevent the pin of the guide element 412 from traveling from the first resting area 508 to the third sloped region 530.

Referring to FIGS. 4-5C together, during operation, when engagement members 406 are in a disengaged configuration, an injection pen 410 is inserted into the universal pen cap 400 in a first axial direction (e.g., a direction extending into the universal pen cap 400). In various embodiments, during insertion of the injection pen 410 into the universal pen cap 400, the injection pen 410 abuts the interface element 414 and causes the interface element 414 to translate along a center longitudinal axis of the universal pen cap 400 in the first axial direction. Causing the interface element 414 to translate along the center longitudinal axis of the universal pen cap 400 in the first axial direction causes the interface element 414 to pull on the engagement members 406 and to push on the guide element 412 in the first axial direction. Pulling on the engagement members 406 causes the engagement members 406 to pivot about connections with the actuating arms 404 and rotate radially inward toward the injection pen 410.

Additionally, in various embodiments, pushing the guide element 412 in the first axial direction causes a pin of the guide element 412 to travel along the first pathway 504 from first resting area 508 along the first sloped region 524 to the first raised portion 522 and into the first recessed portion 512. The first recessed portion 512 is configured to provide a mechanical stop for the pin of the guide element 412 and is configured to prevent further movement of the guide element 412 in the first axial direction. As a result, the stopping the pin of the guide element 412 within the first recessed portion 512 is configured to provide feedback to the user that the injection pen 410 has been fully inserted into the universal pen cap 400.

Moreover, when a user releases the injection pen 410, the biasing elements 408 are configured to cause the interface element 414 to translate along the center longitudinal axis of the universal pen cap 400 in a second axial direction opposite the first axial direction at least some distance. Causing the interface element 414 to translate along the center longitudinal axis of the universal pen cap 400 in the second axial direction causes the interface element 414 to pull on the guide element 412 in the second axial direction. Pulling the guide element 412 in the second axial direction causes the pin of the guide element 412 to travel from the first recessed portion 512 along the second raised portion 526 and into the second resting area 510; furthermore, biasing provided by the biasing elements 408 are configured to at least substantially prevent the pin from leaving the second resting area 510 absent an intentional interaction by a user. The edge formed between the second resting area 510 and the second sloped region 528 is configured to further substantially prevent the pin from leaving the second resting area 510 absent an intentional interaction by the user. Moreover, one of ordinary skill in the art will recognize that the edge at the interface of the first raised portion 522 and the first recessed portion 512 are configured to prevent the pin of the guide element from travelling backward along the first pathway 504. Likewise, the biasing members 704 and the first sloped region 524 provide resistance to the pin of the guide element 412 travelling along the first pathway 504 such that the pin of the guide element 412 does not typically travel along the first pathway 504 unintended. Furthermore, the edge at the interface of the first raised portion 522 and the first recessed portion 512 are configured to provide an audible and haptic feedback click to the user when the injection pen 410 has been sufficiently inserted into the universal pen cap 400. Still further, the edge at the interface of the second raised portion 526 and the second resting area 510 are configured to provide an audible and haptic feedback click to the user indicating that that the engagement members 406 are sufficiently engaged with the injection pen 410. For example, when the pin of the guide element 412 rests within the second resting area 510, the universal pen cap 400 is in an engaged configuration and is configured to clasp the injection pen 410 with the engagement members 406.

Furthermore, when the pin of the guide element 412 rests within the second resting area 510, a subsequent push by a user on the injection pen 410 in the first axial direction causes the guide element 412 to translate in the first axial direction which thereby causes the pin of the guide element 412 to travel along the second pathway 506 from the second resting area 510 along the second sloped region 528 and into the second recessed portion 514. The second recessed portion 514 is configured to provide a mechanical stop for the pin of the guide element 412 and is configured to further prevent movement of the guide element 412 in the first axial direction. As a result, the stopping of the pin of the guide element 412 within the second recessed portion 514 is configured to provide feedback to the user that the injection pen 410 has been pushed a sufficient amount to allow the universal pen cap 400 to move to an unengaged configuration.

Accordingly, when a user releases the injection pen 410, the biasing elements 408 are configured to cause the interface element 414 to translate along the center longitudinal axis of the universal pen cap 400 in the second axial direction. Causing the interface element 414 to translate along the center longitudinal axis of the universal pen cap 400 in the second axial direction causes the interface element 414 to push on the engagement members 406. Pushing on the engagement members 406 are configured to cause the engagement members 406 to pivot about connections with the actuating arms 404 and rotate radially outward away from the injection pen 410. Moreover, causing the interface element 414 to translate along the center longitudinal axis of the universal pen cap 400 in the second axial direction causes the interface element 414 to pull on the guide element 412 in the second axial direction. Pulling the guide element 412 in the second axial direction causes the pin of the guide element 412 to travel along the second pathway 506 from the second recessed portion 514 along the third sloped region 530 to the third raised portion 532 and into the first resting area 508. When the pin of the guide element 412 rests within the first resting area 508, the universal pen cap 400 is in a disengaged configuration and enables removal of the injection pen 410 or a subsequent insertion of an injection pen or re-engagement of injection pen 410. Moreover, the stopping of the pin of the guide element 412 within the first resting area 508 is configured to provide feedback to the user that the injection pen has been disengaged by the engagement member 406 and indicates a user may remove the injection pen 410.

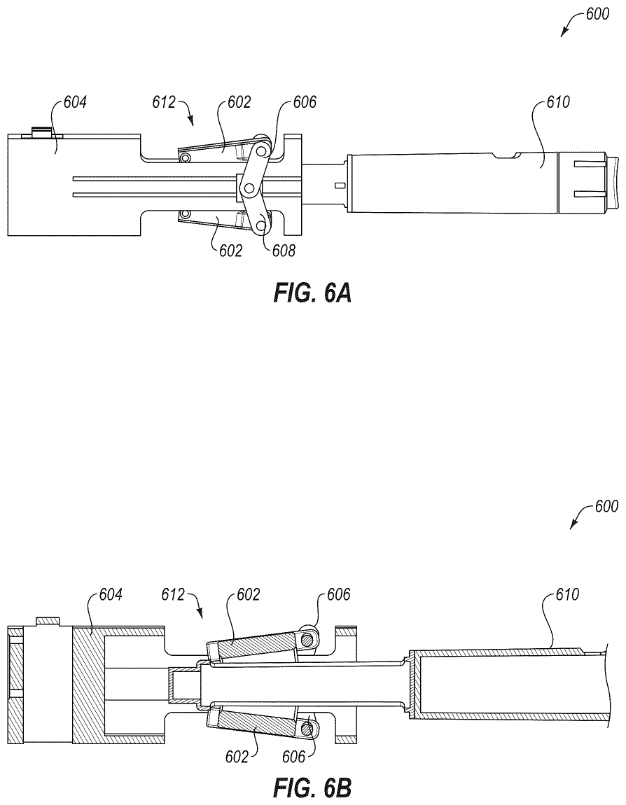

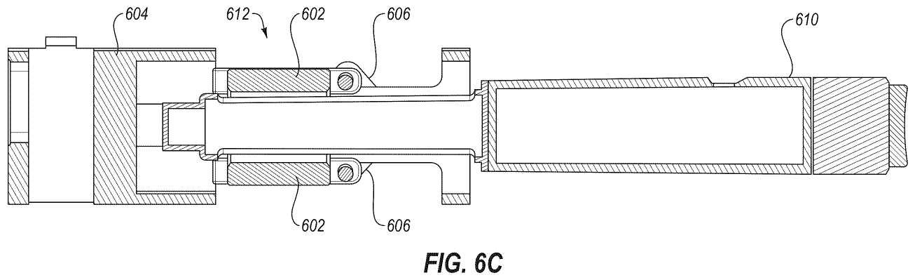

FIG. 6A is a side view of a universal pen cap 600 in a disengaged position according to one or more embodiments of the present disclosure. FIG. 6B is a cross-sectional view of the universal pen cap 600 of FIG. 6A in the disengaged position. FIG. 6C is a cross-sectional view of the universal pen cap 600 of FIGS. 6A and 6B in an engaged position. Referring to FIGS. 6A-6C, in various embodiments, in the engaged position, the universal pen cap 600 is engaged with an injection pen 610. In the disengaged position, the universal pen cap 600 is disengaged from the injection pen 610. In various embodiments, the universal pen cap 600 includes a clamping assembly 612 and body 604. In various embodiments, the clamping assembly 612 includes clasping elements 602 and hinge arms 606/608. In various embodiments, the clasping elements 602 are positioned so as to apply a clamping force to the injection pen 610 and are configured to hold the injection pen 610 in place while in the engaged position. In the embodiment illustrated in FIGS. 6A-6C, the clasping elements 602 are positioned on opposing sides of the body 604 with contact surfaces thereof generally facing one another, such as being circumferentially positioned about one-hundred-eighty degrees from one another relative to the axis of the universal pen cap 600. In embodiments, the clasping elements 602 are adapted to move axially relative to the body 604. In various embodiments, the clamping assembly 612 includes three or more (without limitation) clasping elements 602.

A top end (i.e., the end closest to a closed end of the universal pen cap 600) is positioned radially inward relative to a bottom end (i.e., the end closest to an open end of the universal pen cap 600) of the clasping elements 602. In various embodiments, the bottom end of the clasping elements 602 is rotatably connected to one or more hinge arms 606 at a first longitudinal end of each of the one or more hinge arms 606. Hinge arms 606 are, in turn, rotatably connected to body 604 at a second longitudinal end of each hinge arm 606/608, the second longitudinal end opposite the first longitudinal end of each hinge arm 606/608. In various embodiments, two hinge arms 606, connected to respective clasping elements 602, connect to the body 604 at the same fixed position and to each other at the respective longitudinal ends forming a clasping linkage. In various embodiments, the universal pen cap 600 includes a clasping linkage joining the clasping elements 602 and the body 604 on each lateral side of the clasping elements 602.

In operation, upon insertion of the injection pen 610, the injection pen 610 engages the contact surface of each of the clasping elements 602 at or adjacent to the top end thereof, as can be seen in FIGS. 6A and 6B. As the injection pen 610 is further inserted into the universal pen cap 600, the clasping elements 602 are moved, due to the engagement with injection pen 610, in the axial direction towards the top end of the universal pen cap 600. This axial movement causes rotation of the clasping elements 602 about the fixed position on the body 604 causing an angle of the clamping linkage to reduce, which brings the bottom ends of the clasping elements closer together, which brings more of the contact surface of each of the clasping elements 602 into contact with the injection pen 610, as can be seen in FIG. 6C. In various embodiments, bringing the bottom ends closer together also results in a clamping force being applied to the injection pen 610 while the clasping elements 602 are in the engaged position illustrated in FIG. 6C. Upon removal of the injection pen 610, the clasping elements 602 are pulled away from the top end of the universal pen cap 600 and towards the fixed position which causes the bottom end of the clasping elements 602 to move radially outward, reducing the amount of contact between the contact surface of each of the clasping elements 602 and the injection pen 610 and reducing the clamping force. In various embodiments, each clamping linkage includes a mechanical stop which prevents the hinge arms 606 from rotating too far to prevent the first longitudinal ends from moving too far axially towards the bottom end beyond the fixed position to prevent a clamping force being applied during removal of the injection pen 610. In other embodiments, the universal pen cap 600 includes a mechanical stop that prevents axial movement of the clasping elements 602 beyond a certain position relative to a bottom of the body 604 to prevent a clamping force being applied during removal of the injection pen 610.

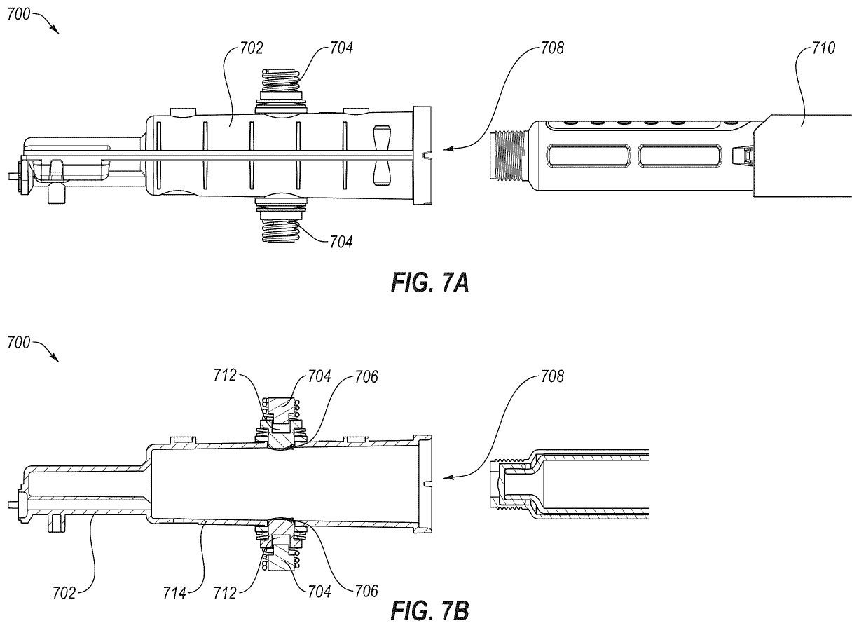

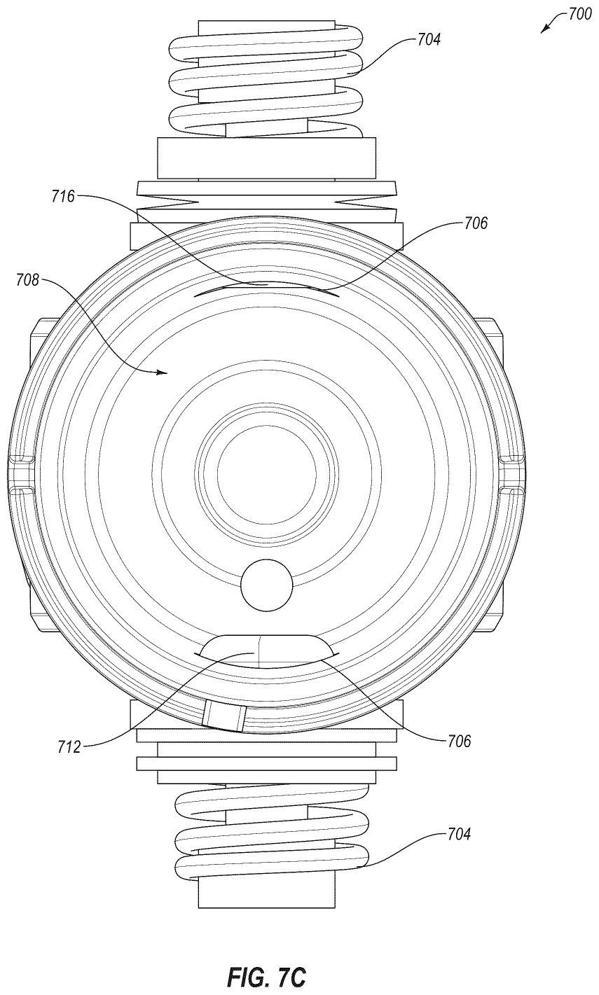

FIG. 7A is a side view of an example universal pen cap 700 adapted to receive an injection pen 710 according to one or more embodiments of the present disclosure. FIG. 7B is a cross-sectional side view of the universal pen cap 700 of FIG. 7A according to one or more embodiments of the present disclosure. Referring to FIGS. 7A and 7B together, in various embodiments, the universal pen cap 700 includes an outer frame element 702 defining one or more first apertures 706 and a second aperture 708. For example, the outer frame element 702 includes an outer wall 714 with the one or more first apertures 706 formed therein and the second aperture 708 is defined by the outer frame element 702 at a longitudinal end thereof. The outer wall 714 may include an annular or a hollow cylindrical shape. In various embodiments, the universal pen cap 700 includes one or more catch members 712 coupled to the outer frame. In various embodiments, the universal pen cap 700 includes one or more biasing members 704, which is operably coupled to one or more catch members 712.

In operation, the one or more catch members 712 are configured to extend through the one or more first apertures 706 and engage an injection pen (e.g., injection pen 710) when an injection pen is inserted into the second aperture 708 of the outer frame element 702. Furthermore, the one or more biasing members 704 are configured to exert a push force on the one or more catch members 712, the push force causing the one or more catch members 712 radially inward toward a center longitudinal axis of the universal pen cap. For example, before insertion of injection pen 710, the one or more catch members 712, as a result of being urged by the one or more biasing members 704, extend through the outer frame element 702 into a cavity defined by the outer frame element 702, the cavity configured to receive an injection pen. Upon insertion of injection pen 710, the one or more catch members 712 are configured to apply an inward radial force on the injection pen 710 while still allowing the push force of a user to continue to insert the injection pen 710. When fully inserted, injection pen 710 abuts an inner surface of the outer frame element 702 while the one or more catch members 712 exert an inward radial force on the injection pen 710 responsive to the biasing members 704 resiliently urging the one or more catch members 712 radially inward. Moreover, the inward radial force exerted by the one or more catch members 712 via the one or more biasing members 704 maintains the injection pen 710 to be maintained within the outer frame element 702 and enables a pull force from a user to remove the injection pen 710 despite the engagement from the one or more catch members.

In various embodiments, the one or more biasing members 704 includes a biasing element (e.g., a spring, without limitation) configured to apply a spring force on the one or more catch members 712 such the spring pushes the catch member to extend through the one or more first apertures 706 and protrude into a cavity of the universal pen cap 700 toward a center longitudinal axis of the universal pen cap 700. In other embodiments, the one or more biasing members 704 includes an elastomer material positioned with respect to the one or more catch members 712 such that, the elastomer material pushes the one or more catch members 712 in a radially inward direction to extend through the first aperture 706 and protrude into a cavity of the universal pen cap 700 toward a center longitudinal axis of the universal pen cap 700. In other embodiments, the one or more catch members 712 includes an elastomer material such that the one or more catch members 712 protrudes through the one or more first apertures 706 and resistibly engage an injection pen (e.g., injection pen 710) that has been inserted into universal pen cap 700 without the need of the one or more biasing members 704.

In one or more embodiments, the one or more catch members 712 include an engagement surface that engages a surface of an injection pen. The engagement surface includes a substantially concave profile when viewed down a longitudinal axis of the universal pen cap 700 so as to substantially compliment the curvature of an injection pen inserted into universal pen cap 700. The one or more catch members 712 include a material having varying roughness and friction. For example, the one or more catch members 712 include rubber, plastic, steel, iron, or an elastomer material. In various embodiments, the catch member 712 includes additional material disposed on the engagement surface of the catch member 712 (e.g., an abrasive material disposed on the engagement surface of the catch member 712, without limitation). In various embodiments the engagement surface includes an irregular pattern, thus enabling the engagement surface to exert more friction when engaged with another surface (e.g., a surface of an injection pen).

FIG. 7C is a front view of a universal pen cap 700 viewed down a center longitudinal axis of universal pen cap 700 through a second aperture 708 showing an example of one or more catch members 712 at different levels of extension through one or more first apertures 706 according to one or more embodiments of the present disclosure. Referring now to FIGS. 7A-7C together, in various embodiments, the first aperture 706 is configured to allow radial translation of the one or more catch members 712 when an outward radial pressure is exerted on a catch member 712 and a biasing member 704 such as, for example, when an injection pen is inserted into second aperture 708.

Still referring to FIG. 7C, when an injection pen (e.g., injection pen 710) is substantially inserted into second aperture 708, the injection pen causes the catch member 712 and biasing member 704 to resistibly retract, as shown by retracted catch member 716. In this position, catch member 712 engages a surface of the injection pen. Further, when there is no injection pen inserted into second aperture 708, the biasing member 704 causes catch member 712 to protrude into a cavity of the universal pen cap 700.

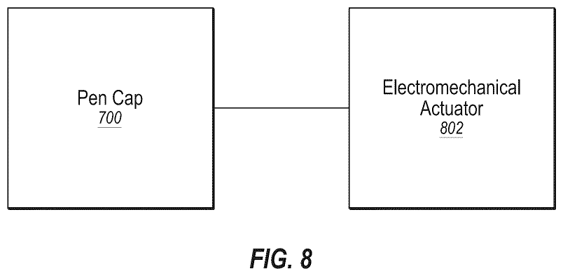

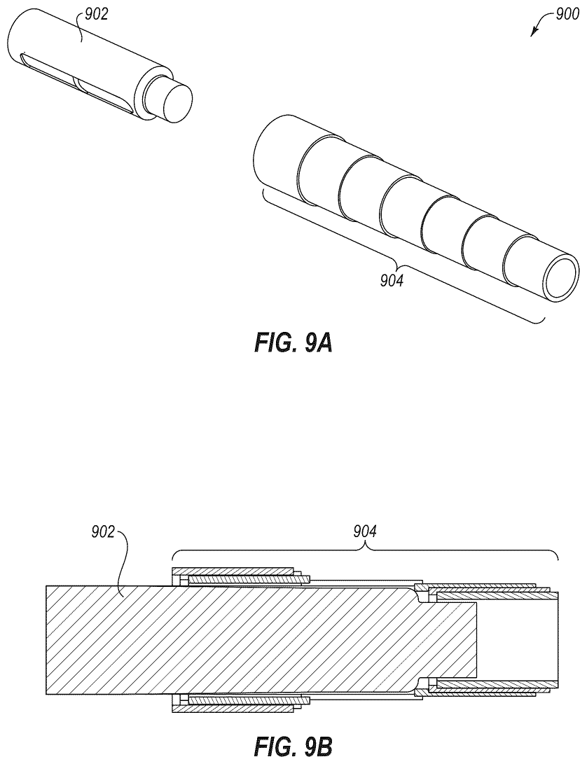

In various embodiments, universal pen cap 700 operably couples with an electromechanical actuator 802 (e.g., as shown in FIG. 8). The electromechanical actuator 802 is configured to actuate one or more catch members 712 such that actuation of the electromechanical actuator 802 causes the one or more catch members 712 to extend through the at least one first aperture 706. In various embodiments, the electromechanical actuator 802 includes a solenoid. In this embodiment, the one or more catch members 712 includes a magnetic material and be disposed within the solenoid such that actuation of the solenoid causes the one or more catch members 712 to translate along a center longitudinal axis of the solenoid. In other various embodiments the electromechanical actuator 802 includes a servo configured to, upon actuation of the servo, translate the one or more catch members through the at least one first aperture 706.

FIG. 9A is a perspective view of a universal pen cap 900 according to one or more embodiments of the present disclosure. FIG. 9B is a cross-sectional side view of a universal pen cap 900 extended over an injection pen according to one or more embodiments. Referring to FIGS. 9A and 9B together, in various embodiments, the universal pen cap 900 includes a tube having a plurality of consecutive segments 904. In various embodiments, the inner diameter of the tube incrementally decrease along a center longitudinal axis of the universal pen cap 900 with each segment of the plurality of consecutive segments 904, each having a respective inner diameter. In various embodiments, each respective inner diameter is substantially constant throughout the respective consecutive segment 904. In some embodiments, the outer diameter of the tube is substantially constant, while the tube is formed with a plurality of consecutive inner right circular cylinders with incrementally decreasing diameters.

Referring to FIG. 9B, in various embodiments the plurality of consecutive segments 904 is configured to telescopically extend in a first longitudinal direction of the universal pen cap 900 and collapse in a second longitudinal direction opposite the first longitudinal direction. In operation, universal pen cap 900 is configured to telescopically extend over an injection pen (e.g., injection pen 902) such that a segment of the plurality of consecutive segments 904 having an appropriate diameter to engage with a surface of the injection pen engages a surface of the injection pen with an interference fit.

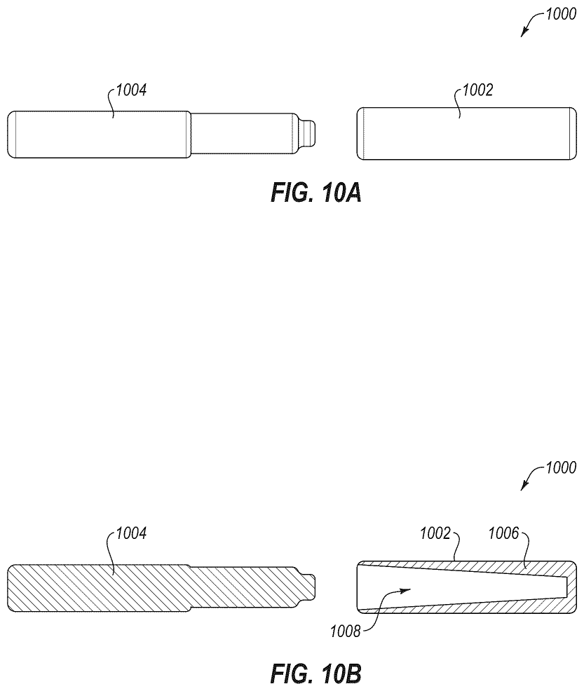

FIG. 10A is a side view of a universal pen cap 1000 according to one or more embodiments of the present disclosure. FIG. 10B is a cross-sectional side view of universal pen cap 1000 of FIG. 10A. Referring to FIGS. 10A and 10B together, in various embodiments, the universal pen cap 1000 includes an outer wall 1006 including an inner frusto-conical cavity 1008 including a frusto-conical shape formed therein. The inner frusto-conical cavity 1008 including a narrowing inner diameter along a center longitudinal axis of the universal pen cap 1000 with a larger diameter end of the frusto-conical shape at an open end of the outer wall 1006. In various embodiments the outer surface 1002 of universal pen cap 1000 includes a substantially equal diameter along the center longitudinal axis of the universal pen cap. In other embodiments, the outer surface 1002 includes a narrowing diameter complimentary to the size and shape of the frusto-conical cavity 1008.

In operation, the universal pen cap 1000 is configured to receive an injection pen 1004 within the frusto-conical cavity 1008 such that a surface of the injection pen 1004 mates with a surface of the frusto-conical cavity 1008 in an interference fit when the injection pen 1004 is inserted into universal pen cap 1000.

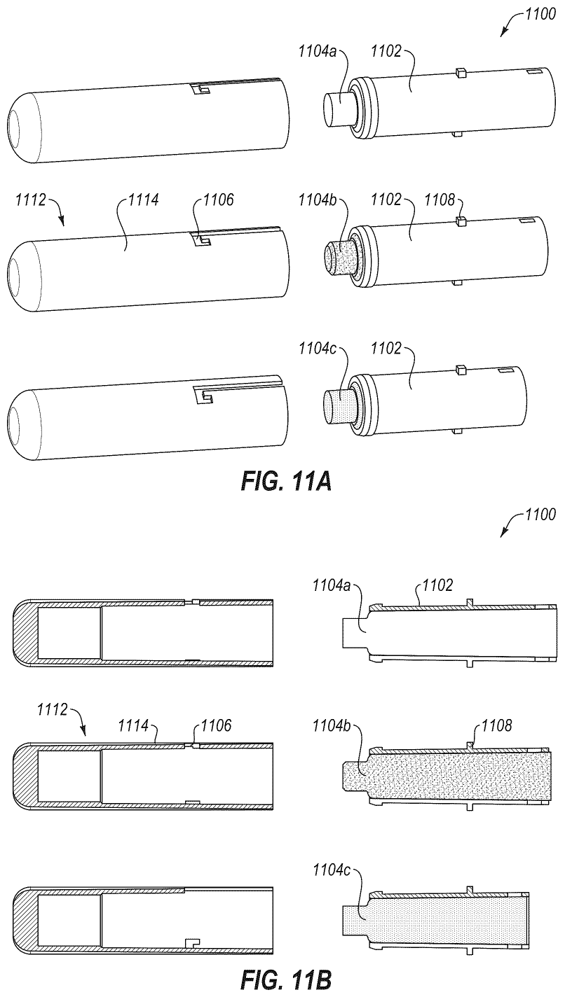

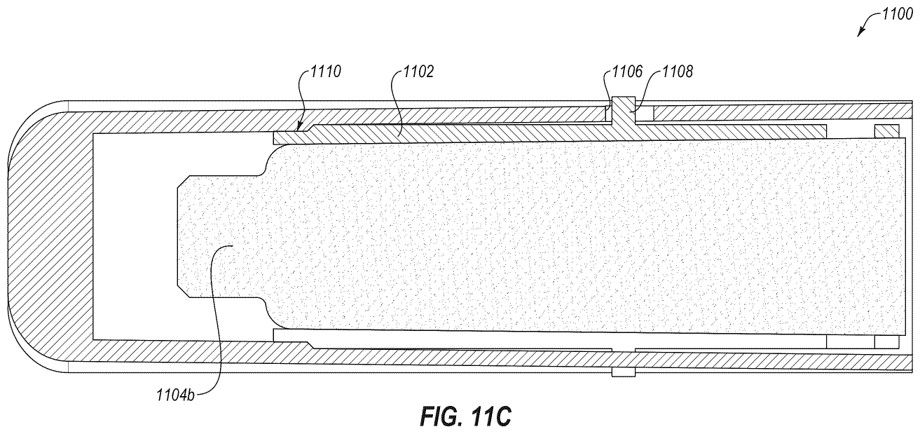

FIG. 11A is a perspective view of a universal pen cap 1100 for receiving a plurality of injection pens 1104a-c according to one or more embodiments of this disclosure. FIG. 11B is a cross sectional side view of the universal pen cap 1100 of FIG. 11A for receiving a plurality of injection pens 1104a-c. Referring to FIGS. 11A and 11B together, universal pen cap 1100 includes an outer shell 1112, a slot 1106, and a sleeve element 1102. Sleeve element 1102 includes a radially extending member 1108.

In various embodiments, the outer shell 1112 includes an outer wall 1114 shaped to at least partially surround a longitudinal end of an injection pen (e.g., any one of injection pens 1104a-c). The outer wall 1114 includes an open end formed therein. The slot 1106 is formed in the outer wall 1114 and extends radially through the outer wall 1114 and also extends axially along a portion of the outer wall 1114. For example, slot 1106 defines a cutout portion from the outer wall 1114 beginning at a longitudinal end of the outer shell 1112 extending at least some distance along a longitudinal axis of the outer shell 1112. In various embodiments, the slot 1106 is formed with a hook shape including a first axial section extending axially in a first direction along the outer wall 1114 from the open end of the outer wall 1114, a circumferential portion extending circumferentially from an end of the first axial section, and a second axial section extending axially in a second direction, opposite the first direction, partially towards the open end of the outer wall 1114. Additionally, sleeve element 1102 is adapted to removably couple to the longitudinal end of an injection pen (e.g., injection pens 1104a-c). In various embodiments, the sleeve element 1102 includes a body and the radially extending member 1108. In various embodiments, the body includes an annular shape formed with an internal cavity adapted to receive an injection pen. Radially extending member 1108 extends radially outward from the body and sized and shaped to slide along the slot 1106 when an injection pen is inserted into the universal pen cap and is adapted to interface with the slot to removably secure the injection pen to the outer shell 1112.

In various embodiments, the sleeve element 1102 is configured to fit over and encompass at least part of a plurality of different injection pen geometries. For example, in various embodiments, sleeve element 1102 includes a deformable material adapted to deform/stretch to conform to a shape of different injection pen geometries, such as an elastomeric material, where the sleeve element 1102 stretches and fits around at least a longitudinal end of an injection pen.

FIG. 11C is a cross-sectional side view of a sleeve element 1102 fitted over an injection pen and removably securing the injection pen to outer shell 1112, according to various embodiments of this disclosure. In various embodiments, sleeve element 1102 is at least partially made from an elastomeric material such that, when an injection pen (e.g., injection pen 1104b) is encompassed by the sleeve element 1102, the elastomeric material creates at least one elastomeric seal 1110 with the outer shell 1112 when the sleeve element 1102 and injection pen are removably secured to the outer shell 1112 by way of the slot 1106 and the at least one radially extending member 1108.

Referring again to FIGS. 11A-C together, in various embodiments, the internal cavity of the outer shell 1112 is formed with a tip receiving section and a body receiving section. The tip receiving section is adapted to receive a top of the injection pen and is formed with an inner diameter that is smaller than that of the body receiving section forming a lip there between. The body receiving section is adapted to receive sleeve element 1102 and a portion of the injection pen covered by the sleeve element 1102. In various embodiments, a portion of the sleeve element 1102 at or adjacent to a leading edge of the sleeve element 1102 includes a diameter that is larger than the tip receiving section. In various embodiments, the interference between the portion of the sleeve element 1102 and the lip acts as a seal. In various embodiments, the interference between the portion of the sleeve element 1102 and the lip acts as a stop, preventing from further insertion of the injection pen into the universal pen cap 1100, which controls a depth of insertion of the injection pen. In various embodiments, the portion of the sleeve element 1102 includes a flange that protrudes radially outward from the body of the sleeve element 1102.

Referring now to FIGS. 11A-C together, in operation, a user may removably coupled the sleeve element 1102 around an injection pen such that the sleeve element 1102 at least partially surrounds a lateral surface of the injection pen. Moreover, when sleeve element 1102 is fitted over an injection pen (e.g., injection pen 1104b), the injection pen, upon insertion into the outer shell 1112 the one or more radially extending members 1108 are configured to slide along the slot 1106 in a first axial direction. When the injection pen 1104b has been fully inserted, the injection pen, and thereby the sleeve element 1102 are turned in the direction of the slot 1106 and then a pull force pulls the one or more radially extending members 1108 into the substantially hook-shaped groove defined by slot 1106. In various embodiments, a biasing member pushes on the inserted injection pen 1104b when the injection pen 1104b has been fully inserted into the outer shell 1112. For example, upon insertion of the injection pen 1104b within the outer shell 1112, a biasing member produces a force in a second axial direction, the second axial direction opposite the first axial direction. Therefore, when the injection pen is inserted and the one or more radially extending members 1108 have reached a longitudinal end of the slot 1106, the injection pen 1104b is turned in the direction of the hook shaped groove defined by the slot 1106 such that, when the push pressure of insertion is released, the biasing member forces the injection pen 1104b, and thereby the sleeve element 1102 and the one or more radially extending members 1108, to translate at least some distance in the second axial direction until the one or more radially extending members abut against a side of the hook-shaped groove defined by the slot 1106. In this position, the sleeve element 1102 via the one or more radially extending member 1108 engages the outer shell 1112 to secure the injection pen 1104b within the outer shell 1112.

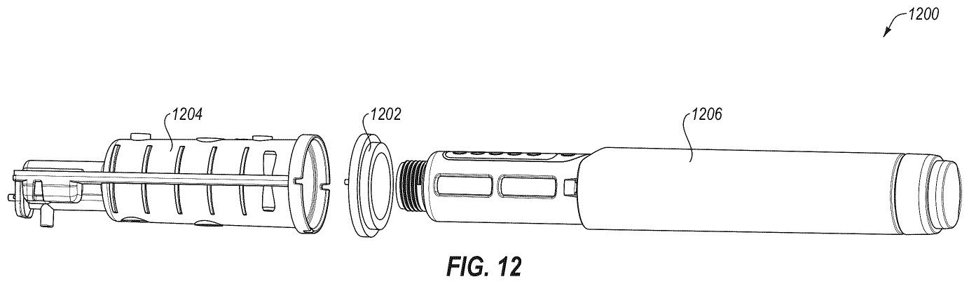

FIG. 12 is a perspective view of a universal pen cap 1200 according to one or more embodiments of this disclosure. In various embodiments, the universal pen cap 1200 includes an outer shell 1204 and a ring element 1202.

In various embodiments, the ring element 1202 is adapted to removably couple to an injection pen such that the ring element circumferentially surrounds the injection pen 1206. In various embodiments, where the ring element 1202 is made from an elastomeric material, the ring element 1202 forms an elastomeric seal with the outer shells 1204 while the ring element 1202 is coupled with an injection pen and while the injection pen 1206, and thereby the ring element 1202, is inserted into the outer shell 1204. The ring element 1202 may be of any material sufficient to form an interference fit with the outer shell 1204. For example, in various embodiment, the ring element 1202 includes an elastomeric material. However, one of ordinary skill in the art will appreciate that Ring element 1202 may be formed of any material sufficient to form an interference fit with the outer shell 1204.

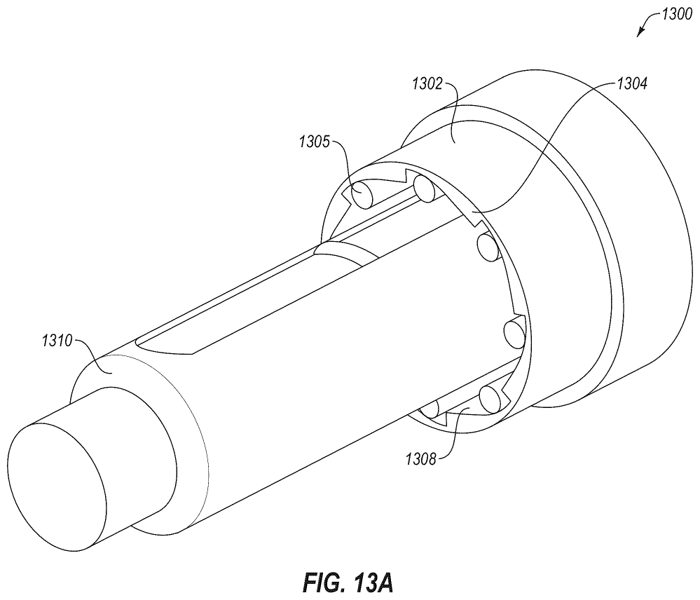

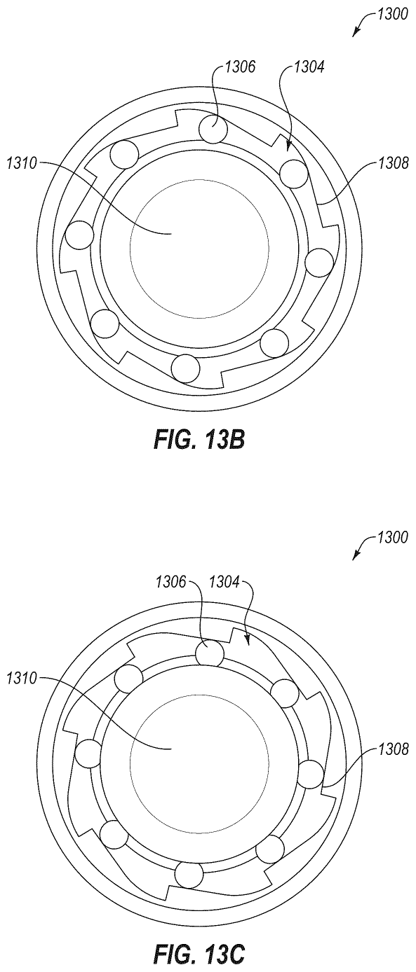

FIG. 13A is a perspective view of a universal pen cap 1300 according to one or more embodiments of the present disclosure. FIG. 13B is a front view of universal pen cap 1300 in a disengaged configuration. FIG. 13C is a front view of universal pen cap 1300 in an engaged configuration. Referring to FIGS. 13A-13C together, in various embodiments, the universal pen cap 1300 includes a collar 1302 and a plurality of sloped protrusions 1304 defining cavities, and a plurality of roller elements 1306 disposed within respective cavities formed by the sloped protrusions 1304. In various embodiments, the sloped protrusions 1304 extend radially inward from the collar 1302, the thickness of each of the sloped protrusions 1304 increasing in a circumferential direction. In various embodiments, the sloped protrusions 1304 are evenly spaced circumferentially around the collar 1302. In various embodiments, the sloped protrusions 1304 define a plurality of sloped surfaces 1308 adapted to contact the roller elements 1306. In various embodiments, the sloped protrusions 1304 define sloped recesses adapted to receive a portion of a respective roller element 1306.

In various embodiments, the universal pen cap 1300 includes at least one roller element retainer chosen from an internal track, a cage, and a flexible collar configured to secure the roller elements 1306 therein. In various embodiments, each roller element 1306 attaches to a protrusion that is configured to run in the track. In various embodiments, a stopper is configured to limit movement of the roller elements 1306.

In various embodiments, each of the roller elements 1306 are disposed adjacent to a respective sloped protrusion adapted to contact an outer radial surface of the injection pen 1310 and respective sloped surfaces 1308 while the injection pen 1310 is inserted into the universal pen cap 1300. In various embodiments, the roller elements 1306 and the sloped protrusions 1304 are adapted for relative movement therebetween in the circumferential direction (herein after “relative rotation”). For example, during operation, an injection pen 1310 is inserted into the universal pen cap 1300 in a first axial direction (e.g., a direction extending into the universal pen cap 1300). Once the injection pen 1310 is inserted into universal pen cap 1300, relative rotation between the collar 1302 and the roller elements 1306 is caused by a user, such as by rotating the collar 1302 in a first rotational direction about a center longitudinal axis of the universal pen cap 1300. The relative rotation between the collar 1302 and the roller elements 1306 causes the roller elements 1306 to each slide or roll along and up a relative sloped surface 1308, thereby causing the roller elements 1306 to translate radially inward toward the center longitudinal axis of universal pen cap 1300 and engage the injection pen 1310 in an interference condition. Likewise, causing relative rotation between the collar 1302 and the roller elements 1306, such as by turning the collar 1302 in a second rotational direction opposite the first rotational direction about the center longitudinal axis of the universal pen cap 1300, causes each of the roller elements 1306 to slide or roll along and down the respective sloped surface 1308, thereby causing the roller elements 1306 to translate radially outward away from the center longitudinal axis of universal pen cap 1300 which causes the roller elements 1306 to reduce the radially interference with the injection pen 1310 and disengage the injection pen 1310. Disengaging the roller elements 1306 from the injection pen 1310 enables a user to remove the injection pen 1310 as well as enable subsequent insertion of injection pen 1310 or a different injection pen into universal pen cap 1300.

In various embodiments the roller elements 1306 include substantially cylindrical rods that are elongated along a longitudinal axis of the universal pen cap 1300. In various embodiments, each cylindrical rod includes a retaining feature configured to hook into a retaining feature (e.g., a track, a cage, between the collar 1302 and a flexible collar, without limitation). In other various embodiments, the roller elements 1306 include substantially spherical bearings. Roller element 1306 includes a durable material such as plastic, iron, steel, rubber, etc.

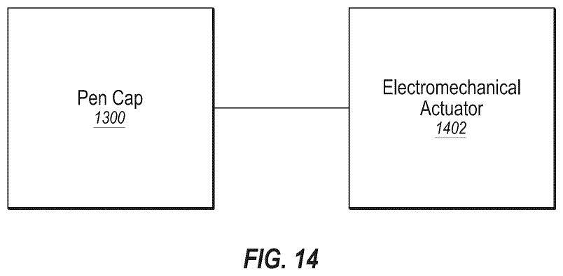

In various embodiments, the universal pen cap 1300 is operably coupled with an electromechanical actuator 1402 (e.g., as shown in FIG. 14). The electromechanical actuator 1402 is configured to cause the relative rotation between the collar 1302 and the roller elements 1306, such as cause rotation of the collar 1302 about a center longitudinal axis of the universal pen cap 1300, both to a disengaged configuration and to an engaged configuration. For example, in various embodiments the electromechanical actuator 1402 includes a solenoid configured to rotate the collar 1302 relative to the roller elements 1306. In yet another example, the electromechanical actuator 1402 includes a servo operably connected to the universal pen cap 1300 such that activation of the servo causes the collar 1302 to rotate about a central longitudinal axis of the universal pen cap 1300.

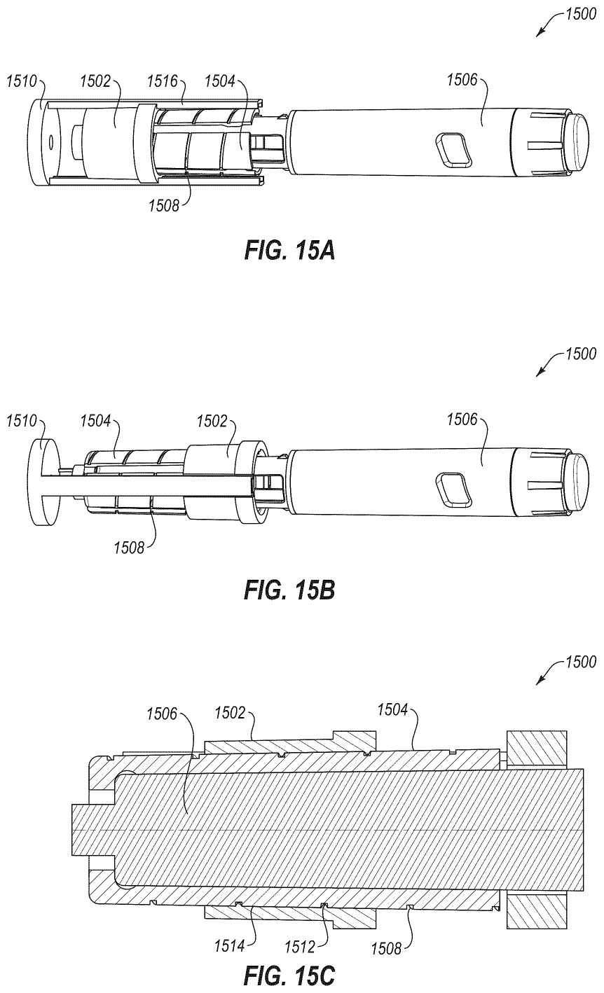

FIG. 15A is a perspective view of a universal pen cap 1500 in a disengaged configuration according to one or more embodiments of this disclosure. FIG. 15B is a perspective view of universal pen cap 1500 in an engaged configuration. FIG. 15C is a cross-sectional side view of universal pen cap 1500 engaged with an injection pen 1506. Referring to FIGS. 15A-15C together, in various embodiments, the universal pen cap 1300 includes a tapered collet 1504, an annular collar 1502, and an outer frame member 1510. In various embodiments, the collet 1504 defines a plurality of recesses 1508 that are formed within radially outer surfaces of the tapered collet 1504 and defines slots extending axially from end of the tapered collet 1504 with a larger diameter towards and end of the tapered collet 1504 with a smaller diameter. In various embodiments, the plurality of recesses is oriented relative to one another in a helical pattern. In various embodiments, the tapered collet 1504 defines a frusto-conical aperture that is configured to receive at least a portion of an injection pen 1506.

In various embodiments, the annular collar 1502 includes a radially inner surface 1514 that defines a central axial aperture configured to receive the tapered collet 1504 therethrough. In various embodiments, a diameter of the radially inner surface 1514 is larger than the smallest diameter of the tapered collet 1504 and smaller than a largest diameter of the tapered collet 1504. In various embodiments, the collar 1502 includes one or more protrusions 1512 that extend radially inward from the radially inner surface of the annular collar 1502. Moreover, the one or more protrusions 1512 are sized, shaped, and positioned to be received into respective recesses 1508 of the plurality of recesses 1508 and adapted to slide along the respective recesses 1508 during operation of universal pen cap 1500. In various embodiments, the translation of the annular collar 1502 in a first axial direction along the tapered collet 1504 causes at least a portion of the tapered collet 1504 to flex radially inward toward a longitudinal axis of the universal pen cap 1500. In various embodiments, the slots facilitate the radially inward flexing of the at least the portion of the tapered collet 1504 to flex. During operation, when universal pen cap 1500 is in a disengaged configuration (e.g., as shown in FIG. 15A), the universal pen cap 1500 receives an insulin pen (e.g., injection pen 1506) through the central axial aperture of tapered collet 1504. A user may then physically turn the annular collar 1502 (and thereby the outer frame member 1510) such that the one or more protrusions 1512 slide along the plurality of recesses 1508 thereby causing the annular collar 1502 to translate along a longitudinal axis of the universal pen cap 1500. The translation of annular collar 1502 toward an engaged configuration (e.g., as shown in FIG. 15B) is configured to cause the tapered collet 1504 to flex radially inward where the diameter of the tapered collet 1504 is greater than the diameter of the radially inner surface 1514 of the annular collar. The tapered collet 1504 is configured to engage the at least partially received injection pen responsive to the radially inward flexing of the tapered collet 1504.

In various embodiments, tapered collet 1504 includes one or more elongated members that extend longitudinally parallel to, and oriented circumferentially around, the central longitudinal axis of the universal pen cap 1500, the one or more elongated members configured to flex radially inward responsive to translation of the annular collar 1502.

In various embodiments, the universal pen cap 1500 includes an outer frame member 1510. The outer frame member 1510 includes one or more elongated guiding members 1516 that extend longitudinally parallel to the central longitudinal axis of the tapered collet 1504 and the universal pen cap 1500. The one or more elongated guiding members 1516 include one or more elongated recesses formed therein extending along the length of elongated guiding member 1516. The one or more elongated guiding members 1516 are configured to receive at least a portion of the annular collar 1502 within the elongated recesses of the elongated guiding member 1516 such that, when the annular collar 1502 is translated along recesses 1508 to an engaged configuration of the universal pen cap 1500, the received portion of the annular collar 1502 slides along the one or more elongated recesses, thereby rotating the outer frame member 1510 with the annular collar 1502. In this way, a user may cause the annular collar 1502 to slide along the one or more elongated recesses as well as the plurality of recesses 1508 and translate along a longitudinal axis of the universal pen cap 1500 by turning the outer frame member 1510. Accordingly, a user may cause the universal pen cap 1500 to go from a disengaged configuration (e.g., as shown in FIG. 15A) to an engaged configuration (e.g., as shown in FIG. 15B), and vice versa by turning the outer frame member 1510.

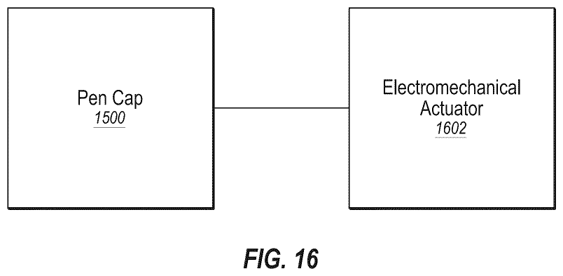

In various embodiments, universal pen cap 1500 includes an electromechanical actuator 1602 (e.g., as shown in FIG. 16) operatively coupled with the actuating components thereof. In particular, in various embodiments, the electromechanical actuator 1602 is configured to actuate one or more of the outer frame member 1510 or the annular collar 1502. In various embodiments, actuation of the electromechanical actuator 1602 causes the outer frame member 1510 and/or the annular collar 1502 to rotate such that the annular collar 1502 translates along a longitudinal axis of the universal pen cap 1500 between open and closed configurations of the universal pen cap 1500. In various embodiments, the electromechanical actuator 1602 includes a servo operably coupled to the outer frame member 1510 and/or the annular collar 1502 such that activation of the servo causes the annular collar 1502 to translate along a longitudinal axis of the universal pen cap 1500. Though discussed in terms of a specific example, one of ordinary skill in the art will appreciate that any conventional electromechanical actuator may be used so long as the electromechanical actuator is configured to cause the annular collar 1502 to translate along a longitudinal axis of the universal pen cap 1500.

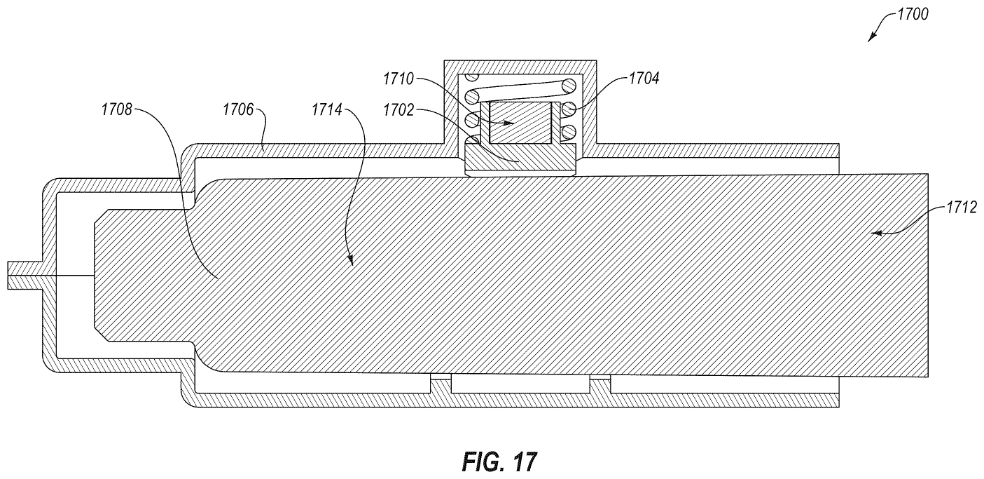

FIG. 17 is a cross-sectional side view of a universal pen cap 1700, according to one or more embodiments of the present disclosure. In various embodiments, the universal pen cap 1700 includes an outer frame element 1706 which defines a side cavity 1710, a body cavity 1714, and an aperture 1712. The universal pen cap 1700 includes an electromechanical actuator 1704 and engagement element 1702 both disposed within the side cavity 1710.

In various embodiments, the engagement element 1702 is positioned in side cavity 1710 in a position chosen from adjoining the body cavity 1714, adjacent to the body cavity 1714, and partially within the body cavity 1714 while in a disengaged position. The electromechanical actuator 1704 is adapted to actuate to cause the engagement element 1702 to protrude into body cavity 1714 and engage an injection pen (e.g., injection pen 1708) when an injection pen is inserted into the aperture 1712. The engagement element 1702 is adapted to contact the injection pen and secure at least a portion of the injection pen positioned in the aperture 1712 therein.

During operation, when the engagement element 1702 is in a disengaged configuration (e.g., before actuation of the electromechanical actuator 802), an injection pen 1708 is inserted into the universal pen cap 1700 in a first axial direction (e.g., a direction extending into the universal pen cap 1700). After the injection pen has been substantially inserted into the universal pen cap 1700, actuation of the electromechanical actuator 1704 causes the engagement element 1702 to translate radially inward toward a center longitudinal axis of the universal pen cap 1700 and toward injection pen 1708. The engagement element 1702 then engages the injection pen 1708 such that the position of injection pen 1708 is maintained within the universal pen cap 1700. The electromechanical actuator 802 is configured to then disengage the engagement element 1702 from the injection pen 1708 and enabling removal of the injection pen 1708.

In various embodiments, electromechanical actuator 1704 includes a solenoid actuator and engagement element 1702 includes a magnet. In at least some of these various embodiments, a compression element is at least partially disposed within the solenoid electromechanical actuator 1704 and adapted to cause the engagement element 1702 to translate along a longitudinal axis of the solenoid into body cavity 1714. In other various embodiments, the electromechanical actuator 1704 includes a servo actuator configured to cause the engagement element 1702 to protrude into body cavity 1714 responsive to the servo electromechanical actuator 1704 being actuated.

In one or more embodiments, the engagement element 1702 includes an engagement surface that engages a surface of an injection pen. In various embodiments, the engagement surface includes a substantially concave profile when viewed down a longitudinal axis of the universal pen cap 1700 so as to substantially compliment the curvature of an injection pen inserted into universal pen cap 1700. In various embodiments, the engagement element 1702 includes a material having varying roughness and friction. In some of these various embodiments, the engagement element 1702 includes at least one of rubber, plastic, steel, iron, or an elastomer material. In various embodiments, the engagement element 1702 includes a different material disposed on the engagement surface of the engagement element 1702. In some of these various embodiments, an abrasive material is disposed only on the engagement surface of the engagement element 1702. In various embodiments the engagement surface includes an irregular pattern, thus enabling the engagement surface to exert more friction when engaged with another surface, (e.g., a surface of an injection pen).

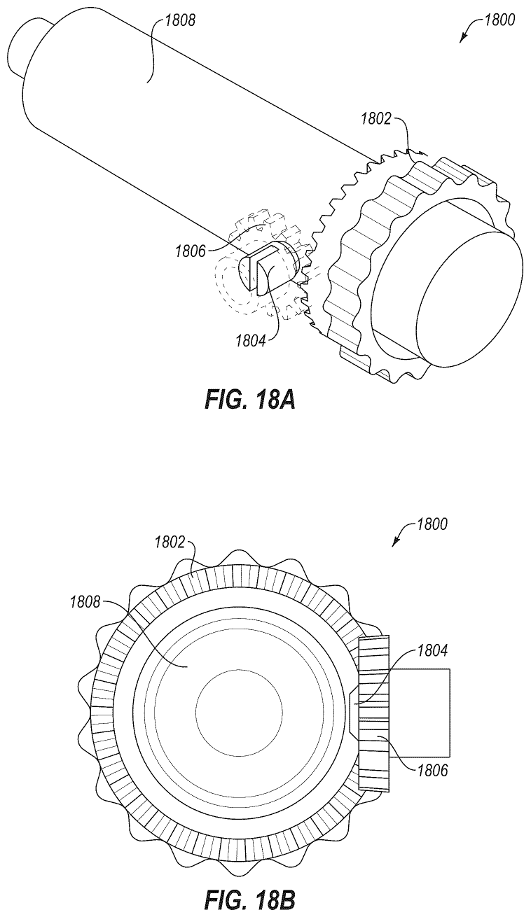

FIG. 18A is a perspective view of a universal pen cap 1800 (with a frame element not shown for clarity) according to one or more embodiments of this disclosure. FIG. 18B is a front view of universal pen cap 1800 (with the frame element not shown for clarity) looking down a longitudinal axis of universal pen cap 1800. Referring to both FIG. 18A and FIG. 18B together, in various embodiments, the universal pen cap 1800 includes an annular drive gear 1802, an annular receiving gear 1806 operably engaged with the annular drive gear, and an engagement member 1804 disposed within a central aperture of the annular receiving gear 1806.

In various embodiments, the annular drive gear 1802 forms a central aperture that is configured to receive at least a portion of an injection pen and at least a portion of the annular drive gear is configured to rotate circumferentially about a longitudinal axis of the universal pen cap 1800. In various embodiments, the annular receiving gear 1806 is configured to rotate about an axis thereof, such as an axis orthogonal to the longitudinal axis of the universal pen cap. The annular receiving gear 1806 is adapted to rotate and cause the engagement member 1804 to translate along an axis thereof which, in various embodiments, is an axis orthogonal to the longitudinal axis of the universal pen cap 1800. Accordingly, rotation of the annular drive gear 1802 engages the operably coupled annular receiving gear 1806, thereby causing the annular receiving gear 1806 to rotate and cause the engagement member to translate toward a center longitudinal axis of the universal pen cap 1800.

In various embodiments the annular drive gear 1802 is configured to receive a portion of an injection pen (e.g., injection pen 1808) such that the annular drive gear 1802 circumferentially surrounds the injection pen. When the annular drive gear 1802 has received at least a portion of the injection pen, the annular drive gear 1802 rotates in a first rotational direction about a central longitudinal axis of the universal pen cap 1800. The annular drive gear 1802 is adapted to rotate to cause the engagement member 1804 to translate in the axial direction thereof, such as an axis orthogonal to the longitudinal axis of the universal pen cap, which thereby causes the engagement member 1804 to engage the injection pen.

In various embodiments, universal pen cap 1800 includes a frame element. In various embodiments, the frame element is a substantially cylindrical frame member received within and rotationally coupled to the central aperture of the annular drive gear 1802. The annular receiving gear 1806 is rotationally coupled to the frame element. In this example, the frame element includes a first aperture in a lateral wall thereof and a second aperture defined at a longitudinal end thereof, where the second aperture is configured to at least partially receive an injection pen (e.g., injection pen 1808). In various embodiments, the engagement member 1804 is configured to translate through the first aperture and engage an injection pen when an injection pen is at least partially received in the second aperture. In various embodiments, either one or both of the annular drive gear(s) 1802 and the annular receiving gear 1806 is coupled to the frame element and adapted to rotate independently from the frame element.

In various embodiments, the frame element is adapted to receive the annular receiving gear 1806 at least partially therein and to receive a first portion of the annular drive gear 1802 therein, the portion being adapted to engage with the annular receiving gear 1806. A second portion of the annular drive gear 1802 is adapted to be exterior to the frame element, accessible to a user, and rotatable relative to the frame element.

In various embodiments, universal pen cap 1800 includes only one gear, (e.g., only annular receiving gear 1806) including the engagement member 1804 being configured to translated by a user by turning the annular receiving gear 1806. In these various embodiments, the universal pen cap 1800 includes a gripping member (e.g., a thumb screw head, abrasive grip, etc.) coupled to the annular receiving gear 1806 to enable ergonomic rotation of the annular receiving gear 1806 by a user. In various embodiments, the annular receiving gear 1806 is rotationally coupled to the frame element with at least a portion thereof positioned outside of the frame element for access thereto by a user.

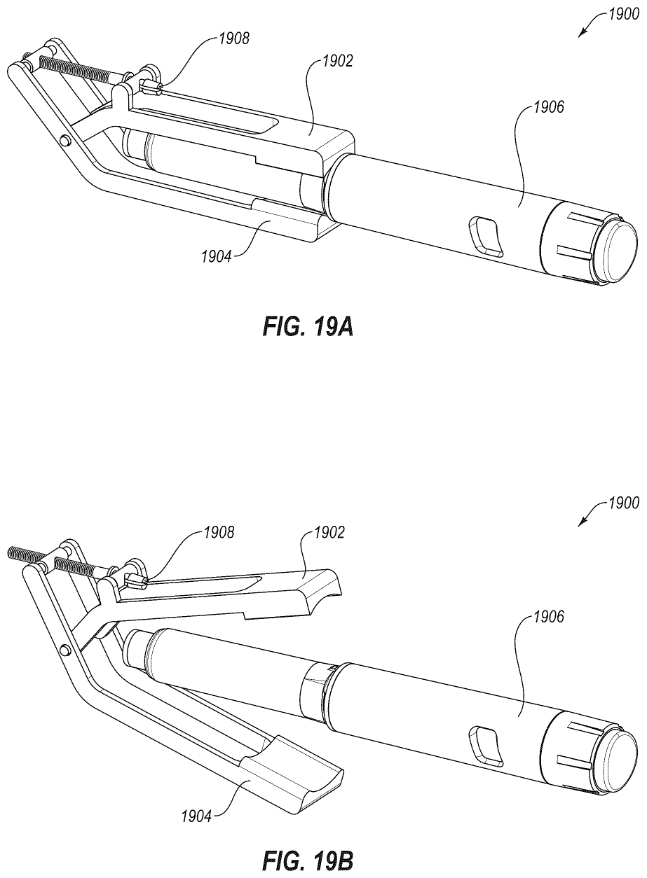

FIG. 19A is a perspective view of a universal pen cap 1900 in an engaged configuration according to one or more embodiments of the present disclosure. FIG. 19B is a perspective view of universal pen cap 1900 in a disengaged configuration. Referring to both FIG. 19A and FIG. 19B together, in various embodiments, the universal pen cap 1900 includes a clamp apparatus including a first arm 1902 and a second arm 1904, the second arm rotatably coupled to the first arm 1902. In various embodiments, the first arm 1902 includes a first body portion and a first coupling portion. The first body portion is adapted to generally extend in an axial direction of the universal pen cap 1900. The first coupling portion extends from the first body portion at an obtuse angle. In the embodiment illustrated, the first coupling portion includes two arms extending from an end of the first body portion. In various embodiments, the first arm 1902 also includes a first engagement portion extending from the first body portion transverse to the first body portion and in a radially inward direction relative to an axis of the universal pen cap 1800. The first engagement portion is adapted to contact the injection pen while the universal pen cap 1900 is in an engaged configuration. In some of these various embodiments, the first body portion, the first coupling portion, and the first engagement portion are formed as a unitary structure.

In various embodiments, the second arm 1904 includes a second body portion and a second coupling portion. The second body portion is adapted to generally extend in an axial direction of the universal pen cap 1900. The second coupling portion extends from the second body portion at an obtuse angle. In the embodiment illustrated, the second coupling portion includes two arms extending from an end of the second body portion. The first coupling portion and the second coupling portion are adapted to rotationally couple. In various embodiments, the second arm 1904 also includes a second engagement portion extending from the second body portion transverse to the second body portion and in a radially inward direction relative to an axis of the universal pen cap 1800. The second engagement portion is adapted to contact the injection pen while the universal pen cap 1900 is in an engaged configuration. In some of these various embodiments, the second body portion, the second coupling portion, and the second engagement portion are formed as a unitary structure.

In various embodiments, the universal pen cap 1900 includes a screw element 1908 configured to move the first arm 1902 and the second arm 1904 between a disengaged configuration an engaged configuration. In the embodiment illustrated, the distal end of the first engagement portion rotationally couples between ends of second engagement portion with the obtuse angles of the first arm 1902 and the second arm 1904 facing each other. In the embodiment illustrated, the first arm 1902 includes a mounting bracket. In this embodiment, the universal pen cap further includes mounting pins with screw holes formed therein adapted to receive the screw element 1908. A first mounting pin is connected to the mounting bracket of the first arm and a second mounting pin is connected to the second coupling portion, such as at a distal end of the second coupling portion relative to the connection between the second coupling portion and the second body portion. In other embodiments, respective mounting pins are formed as a unitary structure with the first arm 1902 and the second arm 1904 respectively. In various embodiments, one of the mounting elements defines a through hole, while the other of the mounting elements defines a threaded-through hole. In various embodiments, the screw element 1908 includes a threaded portion that engages the threaded hole and a locking groove configured to maintain the screw element's 1908 position relative to the through hole along a longitudinal axis of the screw element 1908 while allowing the screw element 1908 to rotate. For example, in the embodiment illustrated, the second mounting pin defines the threaded-through hole and the first mounting pin defines the through hole. In this configuration, the screw element 1908 is configured to translate along its axis relative to the second mounting element and the second arm 1904 and cause the first arm 1902 to rotate relative to the second arm 1904, the rotation being about the point of connection between the first coupling portion and the second coupling portion.

During operation, when the first arm 1902 and the second arm 1904 are in a disengaged position, an injection pen 1906 may be inserted between the first arm and the second arm. While the injection pen 1906 is between the first arm 1902 and the second arm 1904, the screw element 1908 is configured to be rotated, which causes the first arm 1902 rotate about the point of connection relative toward the second arm and swing radially inward toward the second arm 1904. This rotation causes the first arm 1902 and the second arm to engage injection pen 1906. As a result, when the first arm 1902 and the second arm have engaged the injection pen 1906, the universal pen cap 1900 is in an engaged position and is configured to clasp the injection pen 1906 with the first arm 1902 and the second arm 1904.

In various other embodiments, the universal pen cap 1900 includes a torsion spring positioned about a point of connection between the first arm 1902 and the second arm 1904. In some of these various other embodiments, the torsion spring engages the first arm 1902 and the second arm 1904 such that the torsion spring resistibly urges the first arm 1902 and the second arm 1904 inwardly about the point of connection between the first arm 1902 and the second arm 1904 to an engaged configuration of the universal pen cap 1900. In these various embodiments, the first arm 1902 and second arm 1904 include a first and second leverage portion, respectively, that each extends past the point of connection between the first arm 1902 and second arm 1904. The first arm 1902 and the second arm 1904 are configured, in response to user exerting an inward force on the first and second leverage portions against the urging of the torsion spring, rotate about the point of connection to a disengaged configuration. While the inward force is maintained on the first and second leverage portions against the urging of the torsion spring, the universal pen cap 1900 is configured to receive an injection pen (e.g., injection pen 1906) between the first arm 1902 and the second arm 1904. Additionally, the universal pen cap 1900 is configured to in response to the user releasing the inward force on the first and second leverage portions, the universal pen cap 1900 engages the injection pen 1906 as the torsion spring causes the first arm 1902 to rotatably swing radially inward relative to the second arm 1904 and about the point of connection between the first arm 1902 and the second arm 1904. The first arm swinging radially inward causes the first arm 1902, and thereby the second arm 1904, to engage the injection pen 1906 placed there between.

In various embodiments, an electromechanical actuator is coupled to at least one arm chosen from the first arm 1902 and the second arm 1904, such as via the screw element 1908. The electromechanical actuator is configured to rotate at least one arm chosen from the second arm 1904 relative to the first arm 1902 and the first arm 1902 relative to the second arm 1904.

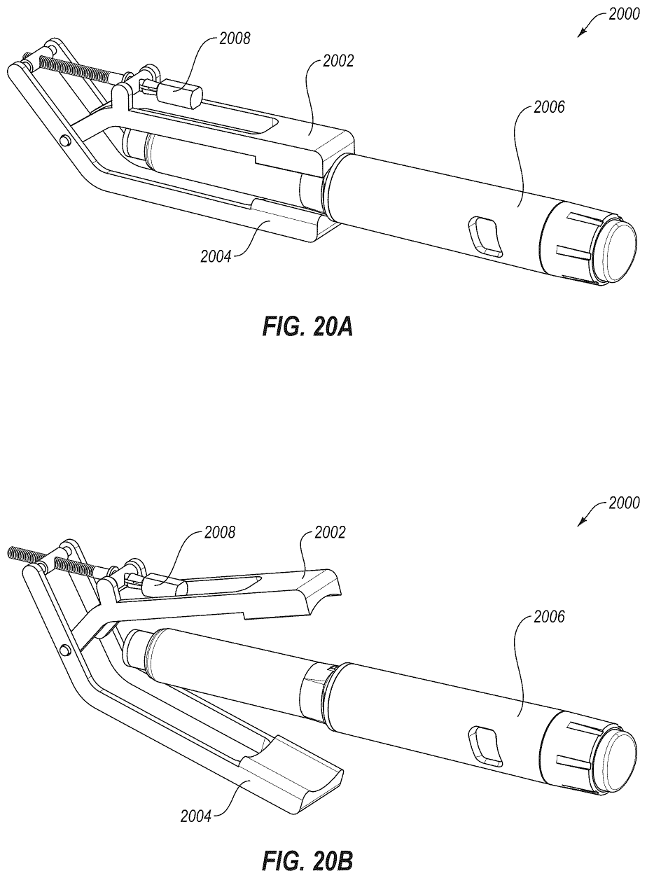

FIG. 20A is a perspective view of a universal pen cap 2000 in an engaged configuration according to one or more embodiments of the present disclosure. FIG. 20B is a perspective view of universal pen cap 2000 in a disengaged configuration. Referring to both FIG. 20A and FIG. 20B together, in various embodiments, the universal pen cap 2000 includes a first arm 2002 and a second arm 2004 rotatably coupled to the first arm 2002. In various embodiments, the universal pen cap 2000 includes an electromechanical actuator 2008 that is coupled to each of the first arm 2002 and the second arm 2004. In various embodiments, the electromechanical actuator 2008 is configured to rotate the second arm 2004 relative to the first arm 2002. The universal pen cap 2000 may be substantially similar to the universal pen cap 1900 but for the inclusion of the electromechanical actuator 2008 and the operations it provides. In particular, in various embodiments, the first arm 2002 and the second arm 2004 includes the various features and connections as the first arm 1902 and the second arm 1904 described above.

In various embodiments, the first arm 2002 and second arm 2004 are configured to receive an injection pen (e.g., injection pen 2006). In some of these various embodiments, the universal pen cap 2000 is configured to receive an injection pen 2006 between the first and second arm 2002, 2004 while in a disengaged configuration (e.g., as shown in FIG. 20B). Upon actuation, the electromechanical actuator 2008 is configured to cause the first arm 2002 to rotate radially inward about a point of connection between the first arm 2002 and the second arm 2004 and relative to the second arm 2004 to an engaged position (e.g., as shown in FIG. 20A). Accordingly, upon actuation of the electromechanical actuator 2008, the first arm 2002 and the second arm 2004 are configured to engage the injection pen 2006 inserted between the first and second arm 2002, 2004.

In various embodiments, electromechanical actuator 2008 may be a solenoid actuator. In other various embodiments, electromechanical actuator 2008 may be a servo actuator. In various embodiments, electromechanical actuator 2008 may be configured to draw power only during period of configuration changes to cause the universal pen cap to transfer or adjust between configuration states (e.g., between an open and closed position as shown in FIGS. 17B and 17A, respectively), such as a transition from a first configuration of the universal pen cap not intended to engage an injection pen to a second configuration intended to engage an injection pen.

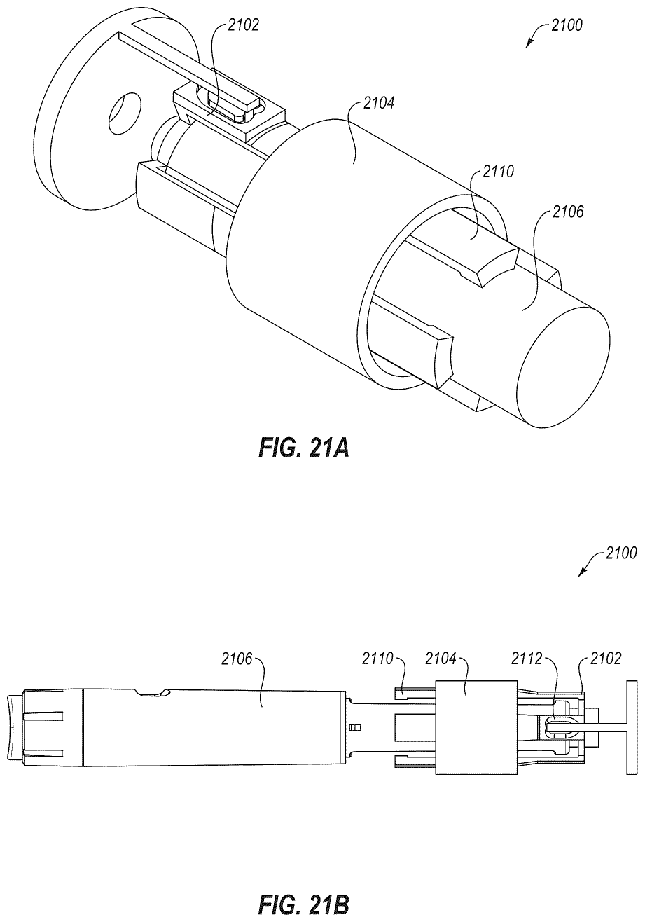

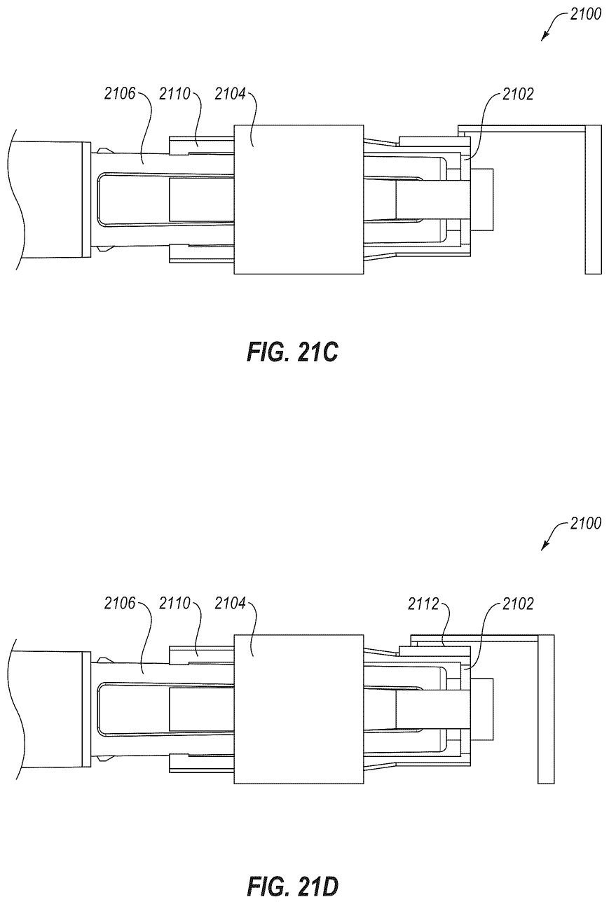

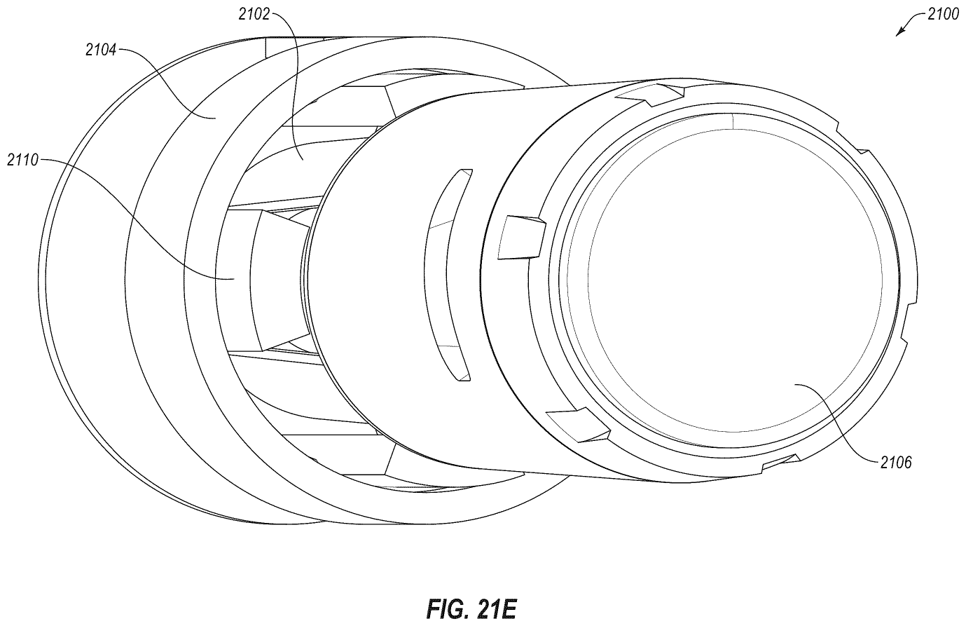

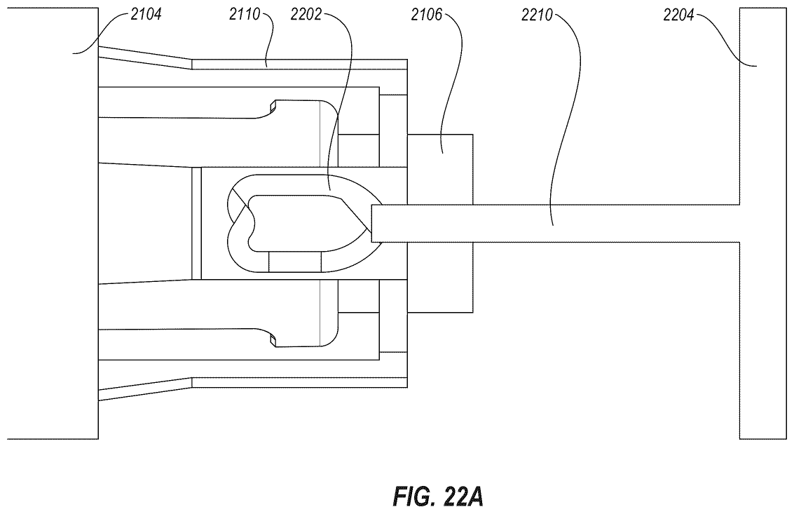

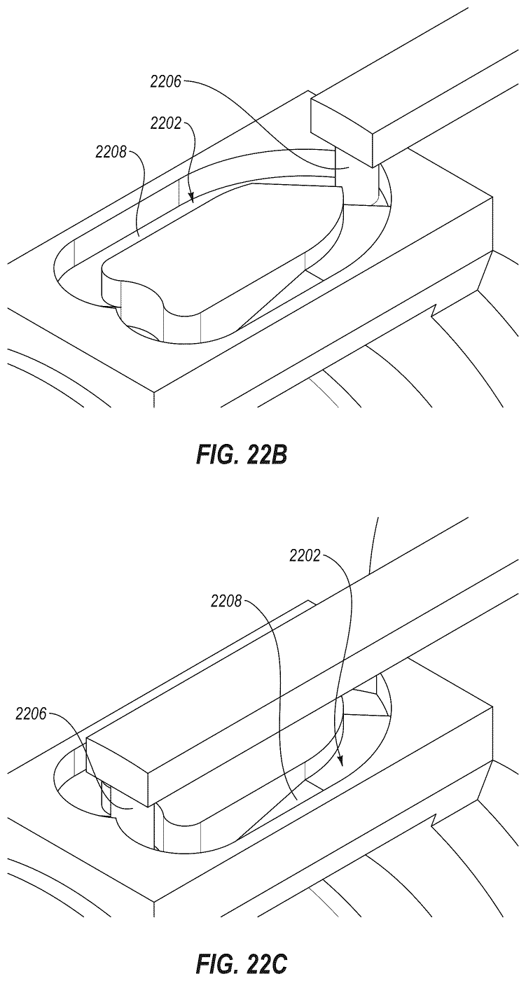

FIG. 21A is a perspective view of a universal pen cap 2100 according to one or more embodiments of this disclosure. FIG. 21B is a side view of universal pen cap 2100. FIG. 21C is a side view of the universal pen cap 2100 of FIGS. 21A and 22B in a disengaged condition. FIG. 21D is a side view of the universal pen cap 2100 of FIGS. 21A-21C in an engaged condition. FIG. 21E is a perspective view of the universal pen cap 2100 of FIGS. 21A-21D in the engaged condition;

Referring to FIGS. 21A-21E, in various embodiments, the universal pen cap 2100 includes a collet 2102 and an outer sleeve 2104. In various embodiments, the collet 2102 includes an end portion and a plurality of elongated members 2110 oriented circumferentially around a center longitudinal axis of the universal pen cap and extending from the end portion and in an axial direction relative to the center longitudinal axis of the universal pen cap 2100, such as extending parallel to the center longitudinal axis. In various embodiments, each of the elongated members 2110 of collet 2102 includes a tapered portion with a tapered width along a longitudinal length thereof. The tapered width is in the radial direction and increases from the end of the tapered portion proximal to the end portion to the end of the tapered portion proximal to an open end of the collet 2102 (distal to the end portion).

The outer sleeve 2104 is disposed about the plurality of elongated members 2110. In various embodiments, the outer sleeve 2104 includes an annular shape, such as a hollow right circular cylinder. The outer sleeve 2104 and the elongated members 2110 are configured for relative movement therebetween with the outer sleeve 2104 sliding relative to the radially outermost surfaces of the elongated members 2110 based on the relative movement therebetween. In various embodiments, the outer sleeve 2104 is configured to engage the elongated members 2110 at a point along a lateral surface of the elongated members 2110. For example, in various embodiments, the elongated member 2110 includes a tapered width with the diameter of an inner radial surface of the outer sleeve 2104 engaging the elongated members where the circumference of the collet is greater than the circumference of the inner radial surface of the outer sleeve 2104, thus causing the plurality of elongated members 2110 to flex radially inward toward a center longitudinal axis of the universal pen cap 2100. Accordingly, when an injection pen (e.g., injection pen 2106) is inserted into universal pen cap 2100, the elongated members 2110 engage with the injection pen responsive to translating the outer sleeve 2104 relative to the elongated members 2110 along a longitudinal axis of the universal pen cap 2100 and thereby causing the elongated member 2110 to flex radially inward toward a center longitudinal axis of the universal pen cap 2100 into an engaged condition, as illustrated in FIGS. 21D and 21E.

In various embodiments, the inner radial surface of the outer sleeve 2104 is a right circular cylinder that includes a diameter that is larger than an outer diameter of the tapered portion at the end proximal to the end portion and smaller than an outer diameter of the tapered portion at the end distal to the end portion. In various embodiments, the inner radial surface of the outer sleeve 2104 is tapered complementary to the tapered elongated members 2110 such that the inner radial surface of the outer sleeve 2104 will engage substantially complimentarily with one or more tapered outer surfaces of the elongated members 2110 as the relative axial position of the outer sleeve 2104 and the universal pen cap 2100 is changed to position of the outer sleeve 2104 to a position further from the end portion along the longitudinal axis of the universal pen cap 2100, thus causing the plurality of elongated members 2110 to flex radially inward toward a center longitudinal axis of the universal pen cap 2100 into the engaged condition. In various embodiments, the taper of the inner radial surface is substantially complementary to the taper of the tapered portion in a first axial position along the collet 2102 with the inner diameters of the outer sleeve 2104 being substantially the same as the outer diameters of the tapered portion. Changing a position of the outer sleeve 2104 at a second position away from the end portion in the axial direction relative to the first position, where the outer diameters of the tapered portion are larger than the inner diameters of the outer sleeve 2104 at corresponding positions, results in radial interference between the outer sleeve 2104 and the elongated members 2110 that causes the radially inward flex of the elongated members.

In various embodiments the universal pen cap 2100 includes a clicker mechanism 2112 configured to move the collet 2102 relative to the outer sleeve 2104 and the outer sleeve 2104 is fixed relative to an outer cover of the universal pen cap 2100. In some of these various embodiments, the outer sleeve 2104 is formed with the outer cover as a unitary structure.

An example clicker mechanism is shown in FIGS. 22A-C. Referring now to FIGS. 22A-C, FIG. 22A is a side view of an example clicker mechanism. FIG. 22B is a perspective view of a clicker mechanism in a closed position. FIG. 22C is a perspective view of a clicker mechanism in an open position. In various embodiments, the clicker mechanism includes a button 2204, a guide element 2210, a biasing member, and a cam element 2202. The button 2204 is positioned at a closed end of the universal pen cap 2100, opposite an open end of the universal pen cap 2100 that is adapted to receive the injection pen 2106. The guide element 2210 extends axially from the button 2204 and is adapted to engage the cam element 2202. In some of these various embodiments, the guide element 2210 is formed as a unitary structure with the button 2204. The biasing member is positioned between the button 2204 and the collet 2102, such as the end portion of the collet 2102. The biasing member is adapted to bias the collet 2102 away from the cap 2100. In various embodiments, the biasing member is a spring, such as a coil spring. The cam element 2202 is adapted to interact with the guide element 2210 to guide a position of the cam element 2202 relative to the button 2204. In various embodiments, the collet 2102 includes the cam element 2202 formed therein, such as in one of the elongated members 2110. In other various embodiments, the cam element 2202 is fixed to the collet 2102. The cam element 2202 includes a groove path 2208 that is adapted to guide the guide element 2210 between two positions formed in the groove path 2208. In some of these various embodiments, the groove path 2208 is substantially similar to the groove path 502 including the first pathway 504, the second pathway 506, and the various components thereof, described above with regards to FIGS. 4-5C. The guide element 2210 includes a pin 2206 (refer to FIGS. 22B and 22C) that is received in the groove path 2208 and is adapted to move along the groove path 2208.

In various embodiments, during operation when an injection pen is inserted into universal pen cap 2100, the universal pen cap 2100 is configured such that when a user applies a push force to the clicker mechanism, and in particular, the button 2204, until a click is heard or felt, the applied force releases the pin 2206 from a first position in the groove path 2208 and allows the biasing member to cause the pin 2206 to move to a second position in the groove path 2208 and cause the elongated members 2110 to translate along a longitudinal axis thereof and into an interference condition with the outer sleeve 2104, thereby causing the elongated members 2110 to flex radially inward toward a center longitudinal axis of the universal pen cap 2100 and engage the injection pen 2106 inserted therein. The universal pen cap 2100 is configured such that when a user applies a subsequent push force to the clicker mechanism until a click is heard or felt, the force causes the pin 2206 to release from the second position and push the pin back to the first position to cause the elongated members 2110 to translate back along the longitudinal axis thereof out of the interference condition with the outer sleeve 2104 such that the inward radial force of the elongated members 2110 is reduced or eliminated to reduce engagement between the elongated members 2110 and the injection pen 2106, thereby allowing a user to remove the injection pen 2106 from the universal pen cap 2100.

In various embodiments, when the pen clicker is in a disengaged configuration (e.g., as shown in FIG. 22C), the universal pen cap 2100 is configured to receive an injection pen 2106 therein and to receive a push force applied to button 2204 by a user, thereby releasing the pin from the first position in the groove path 2208 and allowing the biasing member to push the collet 2102 and the pin 2206 to travel along groove path 2208 to the second position with the universal pen cap 2100 in an engaged configuration (e.g., as shown in FIG. 22B). In various embodiments, pin 2206 is urged inward, such as by a second biasing element, toward a surface of the groove path 2208 such that, when pin 2206 is moved into a recess of the groove path 2208, the user will feel and/or hear a click caused by the pin 2206 striking the surface upon entering a recess in the groove path 2208. When the clicker mechanism is in an engaged configuration, the universal pen cap 2100 is configured to receive a push force applied to the button 2204 by the user until a click is heard or felt and, in response to a release of the button 2204, the pin 2206 travels along the groove path 2208 to an open position. In various embodiments, moving the universal pen cap 2100 from an open position to a closed position, or vice versa, causes the outer sleeve 2104 to translate along a longitudinal axis of universal pen cap 2100 relative to the elongated members 2110 rather than cause the elongated members 2110 to move relative to the outer sleeve 2104.

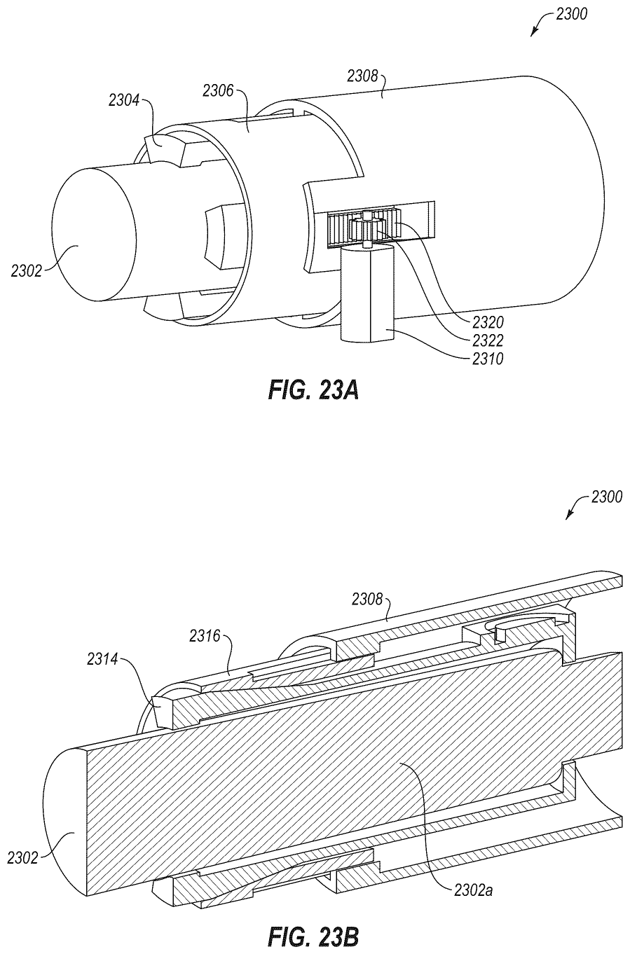

In various embodiments, universal pen cap 2100 includes an electromechanical actuator 2310 as shown by universal pen cap 2300 in FIG. 23A and FIG. 23B. FIG. 23A is a perspective view of a universal pen cap 2300 having an electromechanical actuator 2310, according to one or more embodiments of the present disclosure. FIG. 23B is a cross-sectional perspective view of the universal pen cap 2300 of FIG. 23A. Referring to both FIG. 23A and FIG. 23B together, universal pen cap 2300 may be similar to universal pen cap 2100 but for the inclusion of electromechanical actuator 2310. In particular, in various embodiments, the universal pen cap 2300 includes a collet 2304 that is the same or substantially similar to the collet 2102 and includes an outer sleeve 2306 that is the same or substantially similar to the outer sleeve 2104. In various embodiments, the electromechanical actuator 2310 is configured to slide the outer sleeve 2104 along the radially outermost surfaces of a plurality of elongated members 2314 of the collet 2304. Optionally, the electromechanical actuator 2310 is configured to slide the outer sleeve 2104 along the radially innermost surface of a further sleeve 2308.

In various embodiments, the electromechanical actuator 2310 includes a servo actuator. In some of these various embodiments, the electromechanical actuator 2310 includes a gear 2322 that rotates about a longitudinal axis of the electromechanical actuator 2310. Furthermore, the outer sleeve 2306 includes a ridged receiving portion 2320 disposed, at least partially, on an outer radial surface of outer sleeve 2306 along a longitudinal axis of universal pen cap 2300 and include a series of ridges substantially complimentary to gear 2322 such that rotation of gear 2322 is configured to cause outer sleeve 2306 to translate along a longitudinal axis of universal pen cap 2300 relative to the elongated members 2314. Also depicted is an injection pen 2302

In other various embodiments, electromechanical actuator 2310 includes a solenoid actuator. In some of these various embodiments, the electromechanical actuator 2310 at least partially surrounds outer sleeve 2306. Furthermore, the outer sleeves 2306 include a magnetic material such that actuation of the solenoid electromechanical actuator 2310 surrounding the outer sleeve 2104 causes the outer sleeve 2306 to translate along a longitudinal axis of the universal pen cap 2300. In various embodiments, the electromechanical actuator 2310 is configured to actuate by an actuation device. In various embodiments, the actuation device includes an input chosen from a button and a switch.

In some cases, pen caps (including at least one of the various universal pen cap embodiments disclosed herein) may obscure or encompass varying amounts of an injection pen depending on the length of the injection pen or the length of the pen cap. This may lead to potential issues as injection pens often include important information contained on labels often disposed on a lateral surface of the injection pen, which may be covered or obscured by a universal pen cap. Accordingly, in one or more embodiments of the present disclosure, a universal pen cap includes one or more adjustable floor elements configured to adjust the distance an injection pen may be inserted into a universal pen cap. In various embodiments, the one or more adjustable floor elements floor block element that defines a cavity configured to accommodate a geometry of an injection pen. For example, in various embodiments, the universal pen cap includes a floor block element and a driving mechanism configured to adjust the positioning of the floor block element along a longitudinal axis and within the universal pen cap, thus adjusting the distance an injection pen may be inserted within the universal pen cap. In various embodiments the driving mechanism includes an electromechanical actuator. In other various embodiments, the driving mechanism includes mechanical actuator configured to be actuated by a user.

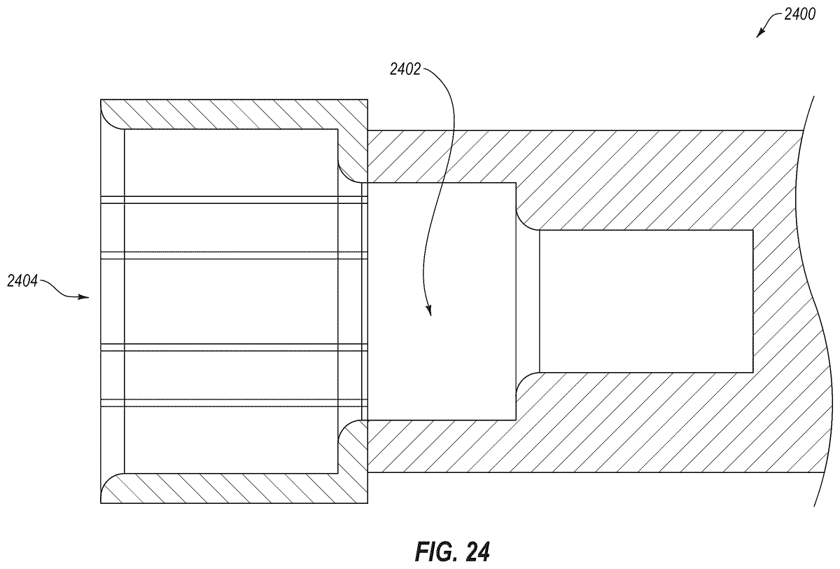

Referring now to FIG. 24, FIG. 24 is a semi-transparent side view of an example floor block element 2400 according to one or more embodiments of the present disclosure. In various embodiments, the floor block element 2400 defines a cavity 2402 and an aperture 2404. In various embodiments, the cavity 2402 is configured to accommodate at least part of the tip of an injection pen when an injection pen is inserted into aperture 2404. In some of these various embodiments, as shown in FIG. 24, the cavity 2402 includes a space defined to account for a needle and/or a needle cover on the tip of an injection pen.

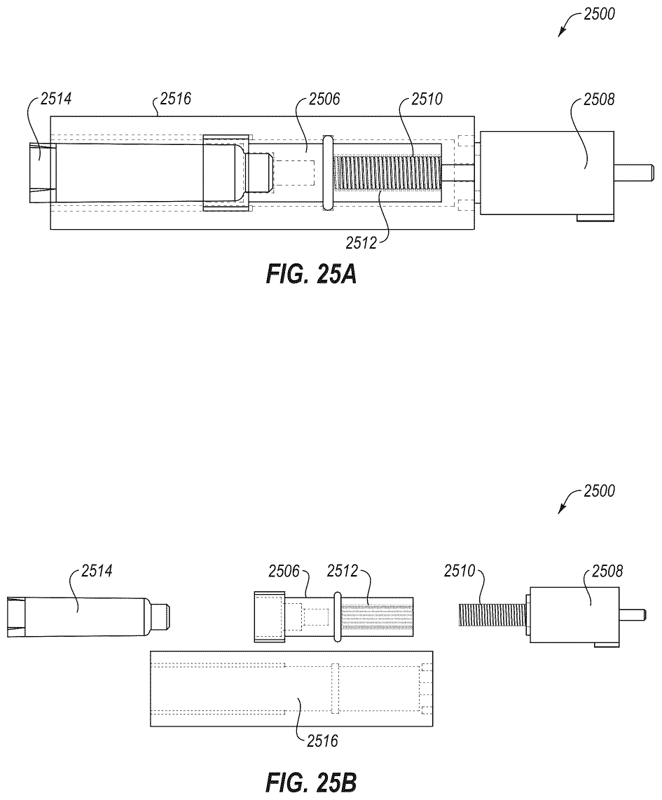

FIG. 25A is a semi-transparent side view of a universal pen cap including adjustable floor elements 2500 according to one or more embodiments of this disclosure. FIG. 25B is a side exploded view of a universal pen cap having adjustable floor elements 2500. Referring to both FIG. 25A and FIG. 25B together, in various embodiments, the adjustable floor elements 2500 includes a floor block element 2400 and an electromechanical actuator 2508. The electromechanical actuator 2508 is operably coupled to a leadscrew element 2510 and floor block element 2400 includes a receiving threaded portion 2512 configured to receive the leadscrew element 2510.

In various embodiments, the electromechanical actuator 1704 is configured to rotate leadscrew element 2510 about a center longitudinal axis of leadscrew element 2510. Furthermore, the leadscrew element 2510 is configured to mate with the receiving threaded portion 2512 such that rotation of leadscrew element 2510 in a first circumferential direction causes translation of the floor block 2506 in the direction of a longitudinal axis of the floor block element 2400 in a first axial direction and rotation of leadscrew element 2510 in a second circumferential direction, opposite the first circumferential direction, causes translation of the floor block 2506 in the direction of a longitudinal axis of the floor block element 2400 in a second axial direction, opposite the first axial direction. In various embodiments, the floor block 2506 is positioned within an outer tube element (e.g., pen cap barrel 2516) such that an injection pen (e.g., injection pen 2514), when inserted into the universal pen cap barrel, is received into the floor block 2506.

Accordingly, a user may adjust the depth at which an injection pen (e.g., injection pen 2514) is insertable into an outer tube element (e.g., pen cap barrel 2516) by translating the floor block 2506 within the pen cap barrel by actuating electromechanical actuator 2508.

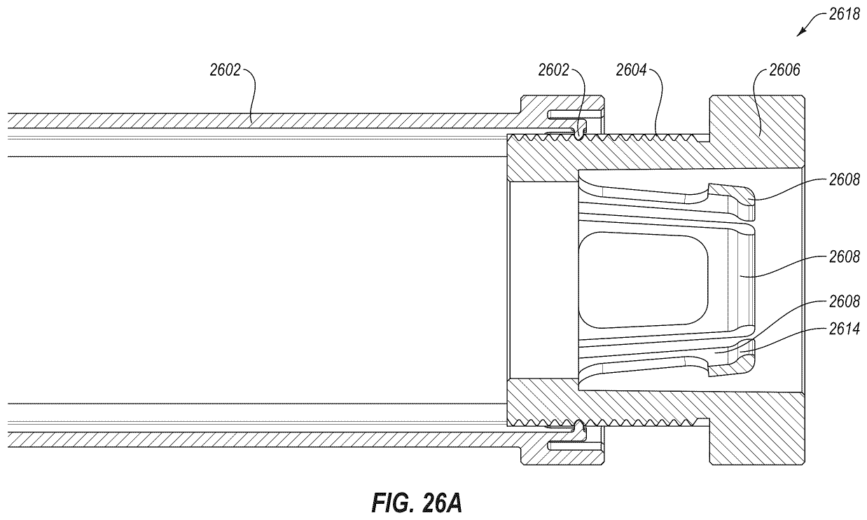

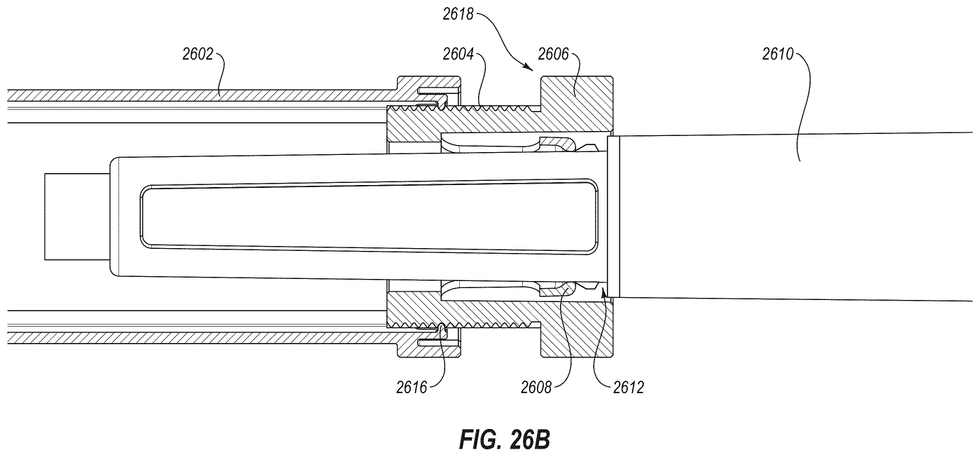

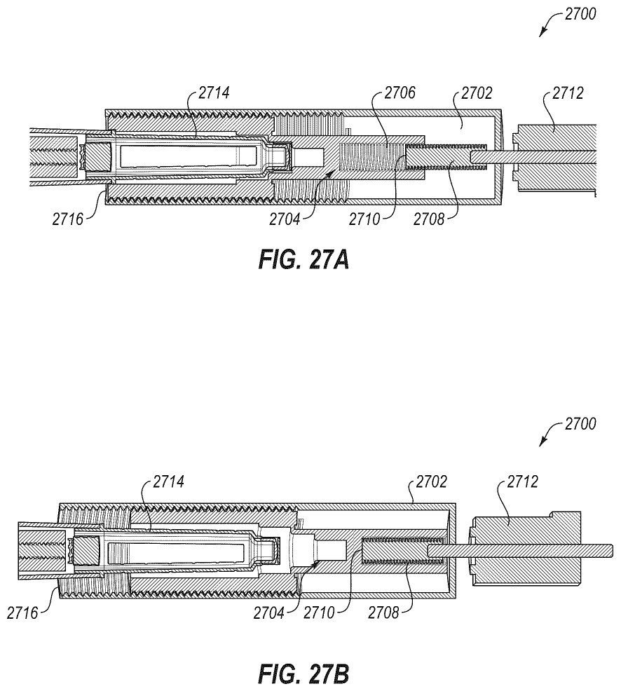

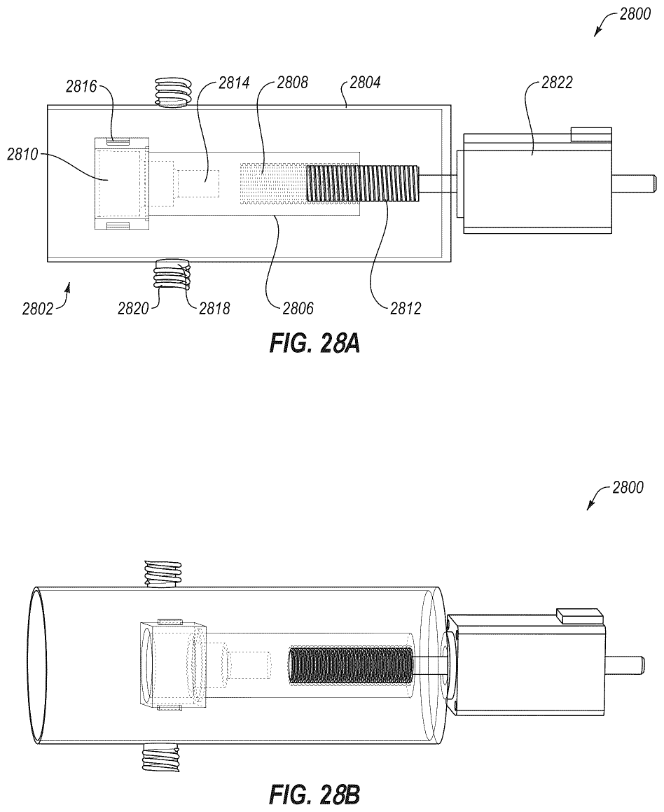

FIG. 26A is a cross-sectional side view of an annular floor block 2606 coupled with a pen cap 2602, according to one or more embodiments of the disclosure. FIG. 26B is a semi-cross sectional side view of an injection pen partially inserted within the annular floor block 2606 shown in FIG. 26A. Referring to both FIGS. 26A and 26B together, in various embodiments, the annular floor block 2606 includes a body, an external flange 2618, and an internal flange. The body includes an annular shape and ridges 2604 disposed circumferentially around at least part of an outer cylindrical surface of the annular shape. In various embodiments, the ridges 2604 extend cylindrically in rings or ring sectors. In other various embodiments, the ridges 2604 define external threads. The external flange 2618 extends radially outward from a first end of the body and defines a lip adapted to abut an end of the pen cap 2602. The internal flange extends radially inward from an inner surface of the body at a second end of the body opposite the first end, the inner surface defining a cavity. The second end being adapted to be received within the end of the pen cap 2602. In various embodiments, the annular floor block 2606 includes one or more flexible arms 2608 disposed within the cavity formed within the body of the annular floor block 2606 and extending adjacent to the inner circumferential surface and in an axial direction away from the second end and towards the first end, which is a direction opposite the pen cap 2602. In various embodiments, the one or more flexible arms 2608 are cantilevered over at least part of the inner circumferential surface. For example, in the embodiment illustrated, the one or more flexible arms 2608 extend axially from an annular surface defined by the internal flange with a cantilevered configuration within the cavity and adjacent to at least part of the inner surface.

In various embodiments, the one or more flexible arms 2608 is sized, shaped, and angled such that the one or more flexible arms 2608 allow at least partial insertion of an injection pen (e.g., injection pen 2610) as shown in FIG. 26B. In some of these various embodiments, the one or more flexible arms 2608 extend primarily in an axial direction and also partially in a radially inward direction toward a center longitudinal axis of the annular floor block 2606 such that, upon insertion on an injection pen, the one or more flexible arms 2608 flex outwardly toward the inner lateral surface of the annular floor block 2606 to accommodate the circumference of the injection pen. In various embodiments, the one or more flexible arms are configured to engage with a feature of an injection pen. As shown in FIG. 26B, injection pens commonly feature a ridge or lip (e.g., injection pen lip 2612) located circumferentially where the pen cartridge meets the pen handle. Accordingly, in various embodiments, the one or more flexible arms 2608 includes a floor block lip 2614 configured to engage the injection pen lip to prevent the one or more flexible arms 2608 from sliding over a surface of the injection pen. In various embodiments, the floor block lip 2614 protrudes inward. In some of these various embodiments, the lip 2614 includes a bulbous shaped cross-section that protrudes radially inward at an end of the one or more flexible arms 2608.

In various embodiments the plurality of ridges 2604 is configured to mate complimentary with a pen cap ridge 2616 such that the pen cap ridge 2616 fits between two ridges 2604 of the plurality of ridges 2604 as shown in FIG. 26A and FIG. 26B. In various embodiments, when the one or more flexible arms 2608 engage with the injection pen lip 2612, in response to continued push force exerted by a user, the annular floor block 2606 translates along a longitudinal axis of the pen cap 2602 until the pen cap 2602 engages with the floor block lip 2618. In some of these various embodiments, when a user has inserted an injection pen (e.g., injection pen 2610) within the annular floor block 2606 to the point where the one or more flexible arms 2608 engage the injection pen lip 2612, in response to continued push force exerted by the user, the plurality of ridges 2604 translate over the pen cap ridge 2616 until the floor block lip 2618 engages with the pen cap 2602. Accordingly, if a user switches from a shorter pen cartridge to a longer pen cartridge, the user would push the injection pen into the pen cap 2602, the injection pen and the one or more flexible arms 2608 engage earlier and continued push force automatically translate the annular floor block 2606 until either the tip of the injection pen engages with the pen cap 2602 or until the pen cap 2602 engages with the floor block lip 2618. Furthermore, if a user were then to change to a longer injection pen, a user may manually pull the annular floor block to a desired length, and in various embodiments, where the ridges 2604 define external threads, the user may manually turn the annular floor block to modify the length of the pen cap 2602 and annular floor block 2606 combination. In various embodiments, the annular floor block 2606 includes a button operably connected to one or more springs configured to release the annular floor block 2606. The one or more springs are adapted and arranged to translate the annular floor block 2606 between the longest and the shortest position of the annular floor block 2606 relative to the pen cap 2602 responsive to actuation of the button.

As noted above, in other various embodiments, the plurality of ridges 2604 define external threads and be in the form of helical ridges extending circumferentially at least once around an outer lateral surface of the annular floor block 2606. The helical ridges are configured to operably mate with the pen cap ridge 2616 such that rotation of the pen cap 2602 or the annular floor block 2606 will cause the pen cap ridge 2616 to thread between adjacent helical ridges/threads to allow the annular floor block 2606 to translate along a longitudinal axis within the pen cap 2602.

FIG. 27A is a cross-sectional side view of a universal pen cap 2700 including an adjustable floor system 2704 in a first position according to one or more embodiments of this disclosure. FIG. 27B shows a cross-sectional side view of the universal pen cap of FIG. 27A including the adjustable floor system 2704 in a second position. In various embodiments, the universal pen cap 2700 includes a housing 2702 configured to receive an injection pen 2714. In various embodiments, the adjustable floor system 2704 includes an internal thread 2706 formed in the housing 2702 and an externally threaded shaft 2708 configured to thread into the internal thread 2706. The externally threaded shaft 2708 includes an end 2710 configured to define an adjustable floor that is movable by threading the externally threaded shaft 2708 into or out of the internal thread 2706. In various embodiments, in response to an injection pen 2714 being inserted into the housing 2702, the externally threaded shaft 2708 is moved within the pen barrel 2702 to adjust an internal length of the housing 2702 from an opening 2716 to the end 2710 of the externally threaded shaft 2708 to accommodate the length of the injection pen 2714 or a length of the portion of the injection pen 2714 configured to be received within a pen cap. In various embodiments, the externally threaded shaft 0708 is configured to lock in place within the housing 2702 utilizing indentations (e.g., teeth or hooks on the inside of the barrel, without limitation). In various embodiments, the universal pen cap 2700 includes an electromechanical driving mechanism 2712, (e.g., a motor, a servo or solenoid mechanism, without limitation). The electromechanical driving mechanism 2712 is configured to turn the externally threaded shaft 2708 and cause the end 2710 to move in an axial direction relative to an axis thereof to change a position of the end 2710 relative to the opening 2716.

FIG. 28A is a cross-sectional side view of a universal pen cap 2800 including an adjustable floor system 2802 according to one or more embodiments of the present disclosure. FIG. 28B shows a cross-sectional side view of the universal pen cap 2800 of FIG. 28A including the adjustable floor system 2802 in a second position. In various embodiments, the adjustable floor system includes an inner tube 2804, a rotating nut 2806, and a threaded stump 2812. The inner tube 2804 is configured to receive the rotating nut 2806 therein. The rotating nut 2806 includes an internal thread 2808 formed at one end and a cavity 2810 formed at an opposing end thereof. The cavity 2810 is configured to receive a portion of the injection pen including a tip thereof. The threaded stump 2812 includes external threads configured to mate with the internal thread 2808 of the rotating nut 2806. The rotating nut 2806 is configured to rotate on the threaded stump 2812 resulting in axial translation of the rotating nut 2806 within the inner tube 2804 along a longitudinal axis thereof. With movement of the rotating nut 2806 within the inner tube 2804, the position of the cavity 2810 is movable to a known position to account for the correct length for receiving a particular injection pen within the universal pen cap 2800. The rotating nut 2806 may translate along the threaded stump 2812 by various mechanisms. In various embodiments, the cavity 2810 includes a cutout 2814 configured to receive the needle and needle cover of the injection pen. In various embodiments, the adjustable floor system 2802 includes magnets 2816 positioned in the rotating nut 2806 (e.g., positioned radially outward from the cavity 2810 and evenly spaced circumferentially about the rotating nut 2806). In various embodiments, the magnets 2816 are permanent magnets. In various embodiments, the adjustable floor system 2802 includes offset stumps 2818 extending radially outward from the inner tube 2804 and coils 2820 wrapped around the offset stumps 2818. In various embodiments, the offset stumps 2818 are evenly spaced circumferentially about the inner tube 2804 (e.g., on the top and bottom of the inner tube 2804, without limitation). In various embodiments, the adjustable floor system 2802 includes an electromechanical driver 2822 configured to rotate the threaded stump 2812. In various embodiments, the combination of the magnets 2816, the coils 2820, and the electromechanical driver 2822 are configured to cause the rotating nut 2806 to translate within the inner tube 2804 in an axial direction thereof by translating the rotating nut 2806 along the threaded stump 2812. While a single internal thread 2808 and threaded stump 2812 combination is illustrated in FIGS. 28A and 28B, in various embodiments, the adjustable floor system 2802 includes multiple internal threads 2808 formed within the rotating nut 2806, each arranged with a corresponding threaded stump 2812.

In various embodiments, the calibration of any of the adjustable floor systems disclosed herein (e.g., adjustable floor system 2704 and adjustable floor system 2802, without limitation) are configured for self-calibration or pre-programmed calibration (without limitation).

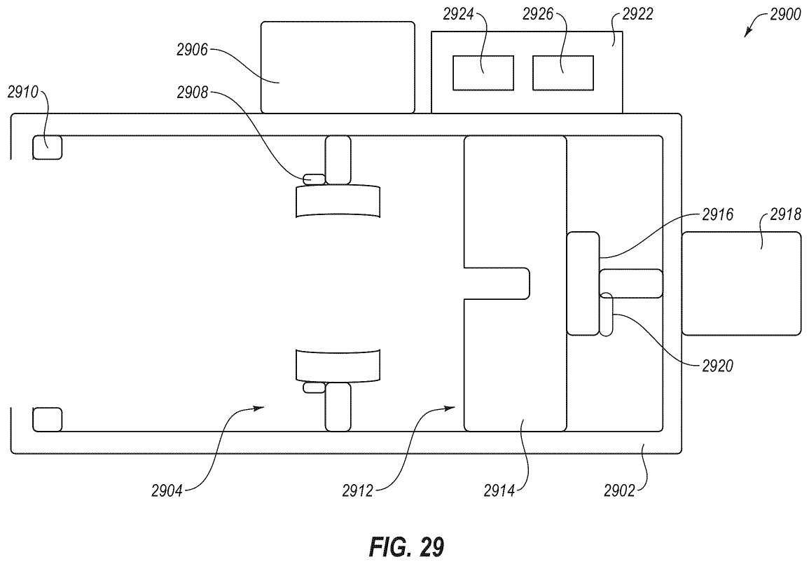

FIG. 29 is a cross-sectional side view of a universal pen cap 2900 including an adjustable floor system 2912 according to one or more embodiments of the present disclosure. In various embodiments, the universal pen cap 2900 includes an inner tube 2902, a clasping mechanism 2904, a clasping mechanism motor 2906, a release sensor 2908, an entry sensor 2910, an insertion sensor 2920, the adjustable floor system 2912, and a controller 2922. The clasping mechanism 2904 is configured to actuate from a disengaged condition to an engaged condition to secure a tip of an injection pen within the inner tube 2902 by applying a compressive force thereto while in the engaged condition. The clasping mechanism motor 2906 is configured to actuate the clasping mechanism 2904 between the disengaged and engaged conditions in response to signals received from the controller 2922.

In various embodiments, the release sensor 2908 is configured to detect a pull by a user attempting to cause relative axial movement between the injection pen and the universal pen cap 2900 in opposing directions (i.e., by sending signals to the controller 2922). In various embodiments, the release sensor 2908 includes a force sensor (e.g., a load cell, strain-gauge, or pressure sensor to detect a force indicative of a user attempting to pull the injection pen from the universal pen cap 2900, without limitation). For example, in various embodiments, the release sensor 2908 is operably coupled to one or more adaptable elements of the universal pen cap 2900 (e.g., the clasping mechanism 2904 and clasping mechanism motor 2906, without limitation) and, in response to detecting a force on the adaptable elements engaging the injection pen that is caused by a user attempting to remove the universal pen cap 2900 from the injection pen, data indicative of an attempted removal of an injection pen from the universal pen cap 2900 that causes the clasping mechanism motor 2906 to actuate the clasping mechanism 2904 from the engaged condition to the disengaged condition to release the injection pen. In various embodiments, the clasping mechanism motor 2906 and the release sensor 2908 are operatively coupled to the controller 2922 and the controller is configured to cause the clasping mechanism motor 2906 to release the injection pen responsive to receiving the data indicative of an attempted removal of the injection pen from the release sensor 2908. In various embodiments, the controller is configured to cause the clasping mechanism motor 2906 to actuate the clasping mechanism 2904 to the disengaged condition responsive to a detected force being greater than a predetermined threshold.

In various embodiments, the release sensor 2908 is configured to detect a force exerted on the injection pen by the one or more adaptable elements (e.g., a clamping force applied by the clasping mechanism 2904, without limitation). For example, in various embodiments, the release sensor 2908 is configured to detect the force with which the clasping mechanism 2904 engages the injection pen and, responsive to detecting the force, communicate data corresponding with that force to the controller 2922.

In various embodiments, the entry sensor 2910 is configured to detect insertion of an injection pen into the universal pen cap 2900 (e.g., insertion within the inner tube 2902, without limitation). In various embodiments, the entry sensor 2910 is positioned adjacent an opening of the inner tube 2902 and is configured the presence of an object such as an injection pen at the opening of the inner tube 2902. Though discussed in terms of particular locations on a universal pen cap, one of ordinary skill in the art will appreciate that, in various embodiments, the entry sensor 2910 is placed anywhere on the universal pen cap 2900 within range of the opening of the universal pen cap 2900 to detect the presence of an injection pen at or adjacent to the mouth/opening of the universal pen cap 2900 (e.g., during insertion or removal).

In various embodiments, the entry sensor 2910 includes a proximity sensor configured to detect an object that comes within a detectable proximal distance thereto. In various embodiments, the proximity detector includes at least one sensor chosen from an optical sensor configured to detect shapes, lights, or movement within an operable distance from the optical sensor, and an infrared sensor configured to detect transmissive or reflective infrared indicators within an operable distance of the infrared sensor. In various embodiments, the entry sensor 2910 is configured to send data to the controller 2922 where the data is indicative of the detection of a detected pen entry event.

In various embodiments, the adjustable floor system 2912 includes an adjustable floor 2914, an adjustable floor actuator 2916 configured to move the adjustable floor 2914 axially within the inner tube 2902, and an adjustable floor motor 2918 configured to cause actuation of the adjustable floor actuator 2916. In various embodiments, the adjustable floor system 2912 includes any combination of one or more aspects of the adjustable floor systems disclosed herein.

In various embodiments, the insertion sensor 2920 is configured to detect when an injection pen has been fully inserted into the universal pen cap 2900. In various embodiments, the insertion sensor 2920 is configured detect a position of an injection pen within the universal pen cap 2900 such that actuation of one or more adaptable elements of the universal pen cap will secure the injection pen within the universal pen cap 2900.

In various embodiments, the insertion sensor 2920 is located at a bottom (e.g., at an inner closed distal end) of the universal pen cap 2900 opposite an opening thereof. In various embodiments, the insertion sensor 2920 is located at or adjacent to the adjustable floor system and is configured to detect a proximity of an end of the injection pen to the adjustable floor system 2912 (e.g., proximity to, such as contact with, an adjustable floor 2914 of the adjustable floor system 2912, without limitation). A person of ordinary skill in the art will appreciate positions and configurations of the insertion sensor 2920 for detection of an injection pen that indicates that the injection has been substantially inserted into the universal pen cap 2900 (e.g., within a predetermined amount of full insertion of a tip of the injection pen within the universal pen cap 2900, without limitation). In various embodiments, the insertion sensor 2920 includes at least one sensor chosen from a proximity sensor and a force sensor. In various embodiments, the proximity sensor is configured to detect the location of an object within a detectable distance of the proximity sensor. In various embodiments, the proximity sensor includes at least one of optical, audio, and infrared sensing capabilities to detect the presence and/or location of a tip of the injection pen positioned within the inner tube 2902. In various embodiments, the force sensor is configured to detect a force applied thereto by the tip of the injection pen that is indicative of a user pushing an injection pen into a fully inserted position within the universal pen cap 2900. In various embodiments, the force sensor includes at least one sensor chosen from a load cell, strain-gauge, or pressure sensor to detect the force of an injection pen being inserted into the universal pen cap 2900.

In various embodiments, the clasping mechanism 2904 is configured to actuate into the engaged condition in response to detection of the injection pen by the insertion sensor 2920 (e.g., detection of the injection pen indicating substantial insertion of the injection pen within the universal pen cap 2900, without limitation). In various embodiments, the insertion sensor is operatively coupled to the controller 2922 and is configured to send data thereto, which data is indicative of at least substantial insertion of the injection pen within the universal pen cap 2900.

In various embodiments, the controller is configured to send instructions to the clasping mechanism motor 2906 to cause the clasping mechanism motor 2906 to actuate the clasping mechanism 2904 into the engaged condition with the injection pen.

In various embodiments, the controller 2922 includes a processor 2924 and memory 2926. The memory storing computer-executable instructions that, when executed, cause the processor 2924 to control the universal pen cap 2900, and in various embodiments, capture data related to the insertion and removal of the injection pen and other data related thereto, such as the actuation of the clasping mechanism 2904 between the engaged and disengaged conditions.

In various embodiments, the processor 2924 is configured to compare data received from the insertion sensor 2920 to at least one threshold value chosen from a force value threshold detected by a force sensor and a proximity value detected by a proximity sensor. In various embodiments, the at least one threshold value is indicative of an injection pen substantially inserted within the universal pen cap 2900.

In various embodiments, any of the entry sensor 2910, the release sensor 2908, and the insertion sensor 2920 further include a sensor configured for detecting at least one condition chosen from contamination, wear, and damage to the universal pen cap 2900 and/or to the injection pen. In various embodiments, the processor 2924 is configured to determine levels of contamination, wear, and damage to the universal pen cap 2900 in response to data received from the sensors. In various embodiments, the processor 2924 is configured to determine an amount of force exerted by the clasping mechanism 2904 necessary to sufficiently engage the injection pen to secure the injection pen within the universal pen cap 2900. For example, different surface frictions of materials used on the exterior of injection pens may require different forces applied by the clasping mechanism 2904 to sufficiently engage the injection pen.

In various embodiments, the processor 2924 is configured to detect operational errors of universal pen cap 2900 (e.g., failure to engage or disengage the injection pen, without limitation). In various embodiments the processor 2924 is configured to cause display of messages or error reports to one or more user interface elements (e.g., a display screen) included in the universal pen cap or to transmit the messages or error reports to a separate user device.

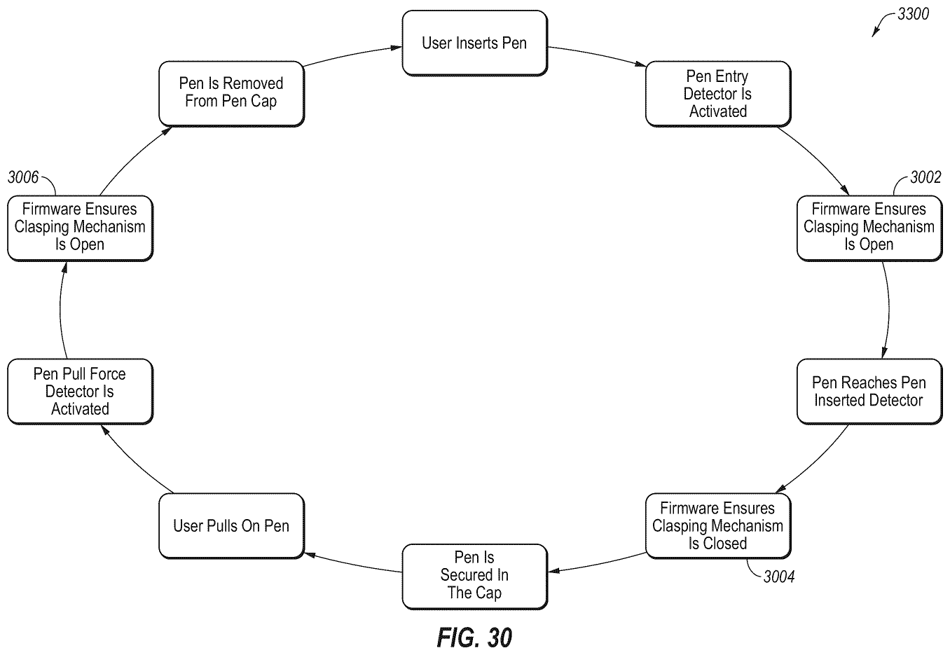

FIG. 30 shows a flow diagram of a method 3000 of operation for a universal pen cap according to one or more embodiments of the present disclosure. In various embodiments, the method includes, in response to detecting insertion of an injection pen into the universal pen cap, ensuring that a clasping mechanism is open at act 3002. In various embodiments, detection of the insertion of the injection pen is performed by a controller 2922 based on signals received from an entry sensor 2910 and comparing the data to predetermined values for that data. In various embodiments, the injection pen is inserted into the universal pen cap by a user of the injection pen.

In various embodiments, the method 3000 includes, in response to the injection pen being substantially inserted into the universal pen cap, clasping the injection pen with a clasping mechanism at act 3004. In various embodiments, act 3004 includes the controller 2922 causing the clasping mechanism to actuate into an engaged configuration. In various embodiments, determining that the injection pen is substantially inserted into the universal pen cap includes comparing data received from an insertion sensor 2920 to one or more predetermined values indicative of a position of the injection pen.

In various embodiments, the method 3000 further includes, in response to detecting relative force between the injection pen and the universal pen cap, releasing the injection pen from the clasping mechanism at act 3006. In various embodiments, detecting relative force between the injection pen and the universal pen cap includes the controller 2922 receiving data from the release sensor 2908 indicative of an axial force applied between the universal pen cap 2900 and the injection pen and comparing the data to a threshold value indicative of a user's attempt to remove the injection pen from the universal pen cap 2900.

Referring again to FIG. 29, in various embodiments, the controller 2922 is configured to calibrate the adjustable floor system 2912 (i.e., self-calibration of the universal pen cap 2900). In various embodiments, the controller 2922 utilizes the data obtained from the he entry sensor 2910, the release sensor 2908, and the insertion sensor 2920 to determine relative information related to the injection pen and the universal pen cap 2900 (e.g., the position of the tip of the injection pen relative to the adjustable floor 2914 with the injection pen fully inserted and the force applied to the injection pen while the clasping mechanism 2904 is in the engaged condition, without limitation). In various embodiments, the controller 2922 is configured to adjust various components of the universal pen cap 2900 in response to the data obtained (e.g., adjust a position of the adjustable floor 2914 to position a tip of the injection pen within a predetermined threshold of the adjustable floor 2914 and to adjust a force applied to the injection pen by the clasping mechanism 2904, such as a force above a first threshold to ensure the injection pen is properly secured and below a second threshold to prevent damage to the injection pen, without limitation).

In various embodiments, the controller 2922 is configured to move the adjustable floor 2914 with the insertion of the injection pen until a predesignated feature is detected. For example, the entry sensor 2910 detects the injection pen and movement thereof, and in response, the controller 2922 causes the adjustable floor motor 2918 to cause the adjustable floor 2914 to move with the injection pen until the predesignated feature reaches a predetermined positioned. In various embodiments, the predesignated feature is a label on the injection pen, and the adjustable floor is moved, for example, until a leading edge of the label is detected, resulting in the label being viewable with the injection pen substantially inserted into the universal pen cap. In various embodiments, the predesignated feature includes at least one of an indentation, protrusion, and marking positioned on the injection pen in a predetermined position on an exterior of the injection pen relative to the placement of the label to ensure the label is not obstructed upon insertion of the injection pen into the universal pen cap. In various embodiments, the predesignated feature includes a step down of the pen handle to the pen cartridge.

The various embodiments, the controller includes a pre-programmed calibration that includes predetermined relative positioning of various components (e.g., adjustable floor position and clasping mechanism position in the engaged and disengaged conditions, without limitation) based on pen geometry. In various embodiments, when a user starts utilizing a medication, they select the medication through a feature on a mobile device within an app or on the universal pen cap display. This then prompts the adjustable floor to move to the correct position within the universal pen cap based on the length of the pen so as to not obstruct the label. In various embodiments, the controller 2922 obtains data related to the pen geometry from an application of an associated user device (e.g., user scans a barcode, such as a linear or matrix (2D) barcode, which identifies the geometry of the injection pen that is provided by the user device to the controller 2922, without limitation).

As discussed herein, a PWD suffering from DFS may sometimes exhibit symptoms that make it difficult to remove or safely cover a medical injection pen. Because of the inherent danger in a PWD being unable to apply a necessary dose or to safely cover a needle, it may be advantageous to enable automatic engagement or disengagement of the universal pen cap from the injection pen to facilitate easier removal or covering of the injection pen, which may further decrease the cognitive strain that a PWD must endure on a daily basis and may help to ensure that a PWD is able to administer a needed dose of drugs/medicine.

In various embodiments, the controller 2922 is configured for data capture events associated with the removal and replacement of the universal pen cap 2900 from and to the injection pen. The data capture events may be indicative of a dosing event, without limitation. In various embodiments, the data capture events include receiving measurements from one or more sensors (e.g., the entry sensor 2910, the release sensor 2908, and the insertion sensor 2920, without limitation) at the controller 2922, comparing the measurements to predetermined threshold values, identifying an event has occurred, and providing the data or derivatives of the data associated with the event to at least one of the user, such as via a display screen, and to an external device associated with the universal pen cap, such as a user device. For example, a universal pen cap event may include, but is not limited to, detecting the entry of an injection pen into a universal pen cap, detecting a pen being inserted into a universal pen cap, detecting a force indicative of an attempt to remove a universal pen cap from an injection pen, detecting a force of one or more adaptable elements used to engage an injection pen, detecting movement or orientation of a universal pen cap, and detecting temperature of a universal pen cap or drugs/medicine within an injection pen inserted into a universal pen cap.

In various embodiments, one or more sensors (e.g., the entry sensor 2910, the release sensor 2908, and the insertion sensor 2920, without limitation) include an accelerometer. In various embodiments, the universal pen cap is configured to automatically wake (e.g., begin using power) responsive to a detected shaking or orientation change of the accelerometer. In some of these various embodiments, one or more adaptable elements included on a universal pen cap are configured to actuate responsive to received data (e.g., a detected shake or orientation change) of an accelerometer. In various embodiments, the universal pen cap is configured to automatically enter a sleep mode (e.g., enter a state of low or no power consumption) responsive to receiving no data from the accelerometer and other sensors for a predetermined period of time.

Additionally, in various embodiments, the universal pen cap also is configured for greater injection pen retention by preventing one or more adaptable elements included in a universal pen cap from being actuated unintentionally, such as from unintended motions like shaking or dropping the universal pen cap. For example, in various embodiments, the accelerometer is configured to detect motions such as drops or incidental or accidental motion of the injection pen that result from the normal day-to-day movements of a user holding or carrying the universal pen cap in contrast to an intentional force applied to the injection pen and the universal pen cap to remove the injection pen. In these embodiments, the accelerometer is configured to ignore the detected motions. Moreover, in various embodiments, the accelerometer is configured to gather information or data pertaining to a drop of the injection pen and universal pen cap, which may be used to assess safety, warranty, and device failure information. In various embodiments, the universal pen cap is configured to display information pertaining to the drop to a user interface device (e.g., a display) included on the universal pen cap and/or to send data pertaining to the drop to a user device associated with the universal pen cap.

In various embodiments, the universal pen cap includes a temperature sensor configured to sense the temperature of the universal pen cap, a temperature of the injection pen, and/or the temperature of drugs/medicine inside the injection pen inserted into the universal pen cap. In various embodiments, the controller is configured to alert a user responsive to the temperature sensor detecting a temperature that is above or below one or more predetermined thresholds. In various embodiments, the alert includes information on the temperature of medication in the injection pen that is inserted into the universal pen cap. In various embodiments, the universal pen cap includes a display on an exterior thereof configured to display usage patterns such as, for example, the temperature of the universal pen cap or the temperature of the drugs/medicine inside the injection pen inserted into the universal pen cap over a period of time.

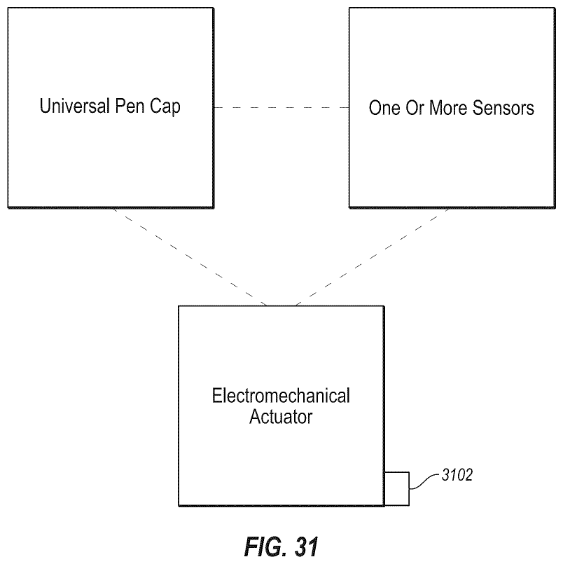

FIG. 31 shows a block diagram of a system 3100. System 3100 includes a universal pen cap, one or more sensors, and an electromechanical actuator.

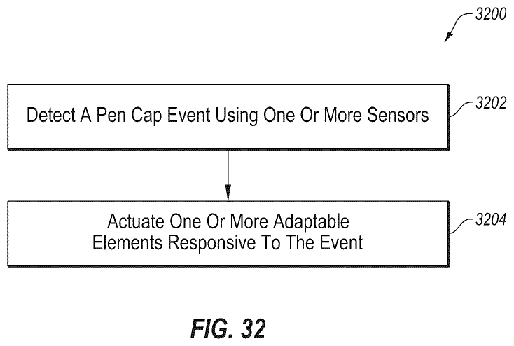

FIG. 32 shows a flow diagram of a method 3200 of an operation of a universal pen cap (e.g., any of the universal pen caps of the present disclosure) according to one or more embodiments of the present disclosure. In operation 3202 of the method 3100, the universal pen cap is configured to detect a universal pen cap event using one or more sensors. In operation 3204 of the method 3200, the universal pen cap actuates one or more adaptable elements responsive to the detecting the universal pen cap event.

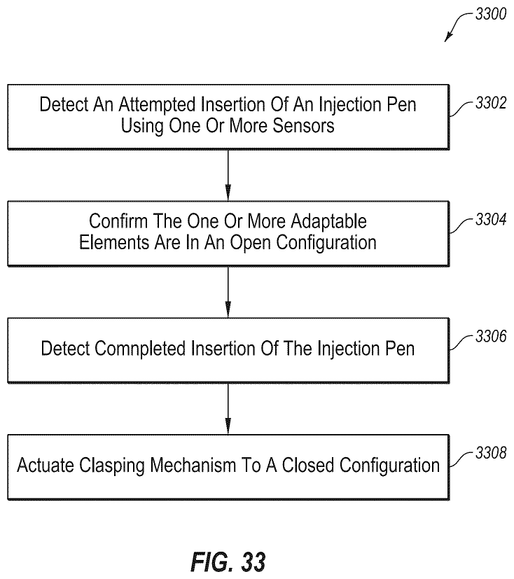

FIG. 33 shows a flow diagram of a method 3300 of actuating one or more adaptable elements of a universal pen cap responsive to detecting an insertion of an injection pen into the universal pen cap. In operation 3302, the universal pen cap detects an attempted insertion of an injection pen using one or more sensors. In operation 3304, the universal pen cap confirms that one or more adaptable elements are in a disengaged configuration. In various embodiments, responsive the one or more sensors detecting that the one or more adaptable elements are in an engaged configuration and are not engaged with an injection pen, the universal pen cap causes the one or more adaptable elements to move to a disengaged configuration to enable subsequent insertion of the injection pen. In operation 3306, the pen cap detects a completed insertion of the injection pen into the universal pen cap. In various embodiments, a completed insertion includes the injection pen being inserted such that the tip of the injection pen is within a predetermined distance of a closed longitudinal end of the universal pen cap. In some of these various embodiments, a completed insertion includes the injection pen being inserted such that the tip of the injection pen is within a floor block element (e.g., any of the floor block elements of the present disclosure, without limitation). Moreover, a completed insertion includes the injection pen being placed within the universal pen cap to substantially conform to a predetermined positioning. In operation 3308, the universal pen cap actuates the one or more adaptable elements to an engaged configuration. In various embodiments, upon insertion of an injection pen into a universal pen cap (e.g., a universal pen cap discussed herein, without limitation), the universal pen cap actuates the one or more adaptable elements, causing them to removably engage the injection pen.

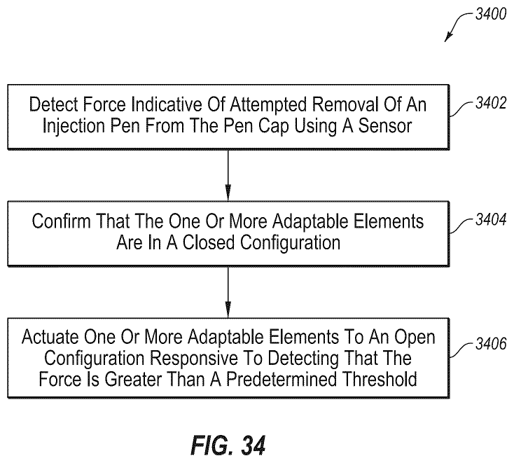

FIG. 34 shows a flow diagram of a method 3400 of actuating one or more adaptable elements of a universal pen cap responsive to a detected force. In various embodiments, method 3400 is performed by a device or system, such as a universal pen cap for medicinal injection pens disclosed herein. In operation 3402, the universal pen cap detects a force indicative of an attempted removal of an injection pen from the universal pen cap using a sensor. In various embodiments, a sensor detects the force created by a user attempting to pull a universal pen cap from an injection pen where the universal pen cap is engaged with the injection pen. In operation 3404, the universal pen cap confirms that the one or more adaptable elements are in an engaged configuration. For example, a sensor detects that the one or more adaptable elements are engaged with an injection pen that has been inserted into the universal pen cap. In other various embodiments, the sensor detects that the one or more adaptable elements are in an engaged configuration but are not engaged with an injection pen. In operation 3406, the universal pen cap actuates the one or more adaptable elements to a disengaged configuration responsive to detecting that the force is greater than a predetermined threshold. In various embodiments moving to a disengaged configuration causes the one or more adaptable elements to no longer engage with the injection pen. In various embodiments, a disengaged configuration causes the one or more adaptable elements to reduce the force exerted on the injection pen to reduce engagement with the injection pen such that a user is able to remove the injection pen. In various embodiments the universal pen cap performs an additional operation to check to confirm whether the one or more adaptable elements have moved to a disengaged configuration. In various embodiments, the universal pen cap detects a removal of an injection pen after the one or more adaptable elements have moved to a disengaged configuration.

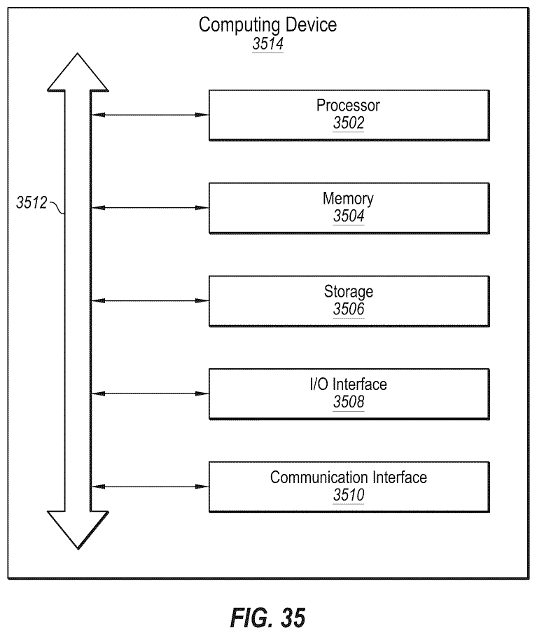

FIG. 35 is a block diagram of an exemplary computing device 3514 that configured to be utilized within and/or as a portion of the universal pen caps disclosed herein. In various embodiments, the computing device 3514 is configured (e.g., programmed, without limitation) to perform one or more of the processes described above. One will appreciate that one or more computing devices may implement the computing device 3514. The computing device 3514 includes a processor 3502, a memory 3502, a storage device 3506, an I/O interface 3508, and a communication interface 3510, communicatively coupled by way of a communication infrastructure 3512. While an exemplary computing device is shown in FIG. 35, the components illustrated in FIG. 35 are not intended to be limiting. Additional or alternative components may be used in other various embodiments. Furthermore, in various embodiments, the computing device 3514 includes fewer components than those shown in FIG. 35. Components of the computing device 3514 shown in FIG. 35 will now be described in additional detail.

In one or more embodiments, the processor 3502 includes hardware for executing instructions, such as those making up a computer program. As an example, and not by way of limitation, to execute instructions, the processor 3502 retrieves (or fetch) the instructions from an internal register, an internal cache, the memory 3504, or the storage device 3506 and decode and execute them. In one or more embodiments, the processor 3502 includes one or more internal caches for data, instructions, or addresses. As an example and not by way of limitation, the processor 3502 includes one or more instruction caches, one or more data caches, and one or more translation lookaside buffers (TLBs). Instructions in the instruction caches may be copies of instructions in the memory 3504 or the storage device 3406. In various embodiments, the memory 3504 is used for storing data, metadata, and programs for execution by the processor(s). The memory 3504 includes one or more of volatile and non-volatile memories, such as Random Access Memory (“RAM”), Read-Only Memory (“ROM”), a solid state disk (“SSD”), Flash memory, Phase Change Memory (“PCM”), or other types of data storage. In various embodiments, the memory 3504 is internal or distributed memory.

The storage device 3506 includes storage for storing data or instructions. As an example and not by way of limitation, storage device 3506 includes a non-transitory storage medium described above. In various embodiments, the storage device 3506 includes at least one storage chosen from a hard disk drive (HDD), a floppy disk drive, flash memory, an optical disc, a magneto-optical disc, magnetic tape, or a Universal Serial Bus (USB) drive or a combination of two or more of these. In various embodiments, the storage device 3506 includes removable or non-removable (or fixed) media, where appropriate. The storage device 3506 may be internal or external to the computing device 3514. In one or more embodiments, the storage device 3506 is non-volatile, solid-state memory. In other various embodiments, the storage device 3506 includes read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these.

The I/O interface 3508 allows a user to provide input to, receive output from, and otherwise transfer data to and receive data from computing device 3514. In various embodiments, the I/O interface 3508 includes a mouse, a keypad or a keyboard, a touch screen, a camera, an optical scanner, network interface, modem, other known I/O devices or a combination of such I/O interfaces. The I/O interface 3508 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 3508 is configured to provide graphical data to a display for presentation to a user. The graphical data may be representative of one or more graphical user interfaces and/or any other graphical content as may serve a particular implementation.

The communication interface 3510 includes hardware, software, or both. In any event, the communication interface 3510 provides one or more interfaces for communication (such as, for example, packet-based communication) between the computing device 3514 and one or more other computing devices or networks. As an example and not by way of limitation, the communication interface 3510 may include a network interface controller (NIC) or network adapter for communicating with an Ethernet or other wire-based network or a wireless NIC (WNIC) or wireless adapter for communicating with a wireless network, such as a WI-FI.

Additionally or alternatively, in various embodiments, the communication interface 3510 is configured to facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the communication interface 3510 may facilitate communications with a wireless PAN (WPAN) (such as, for example, a BLUETOOTH® WPAN), a WI-FI network, a WI-MAX network, a cellular telephone network (such as, for example, a Global System for Mobile Communications (GSM) network), or other suitable wireless network or a combination thereof.

Additionally, the communication interface 3510 is configured to facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

The communication infrastructure 3512 includes hardware, software, or both that couple components of the computing device 3514 to each other. As an example and not by way of limitation, the communication infrastructure 3512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

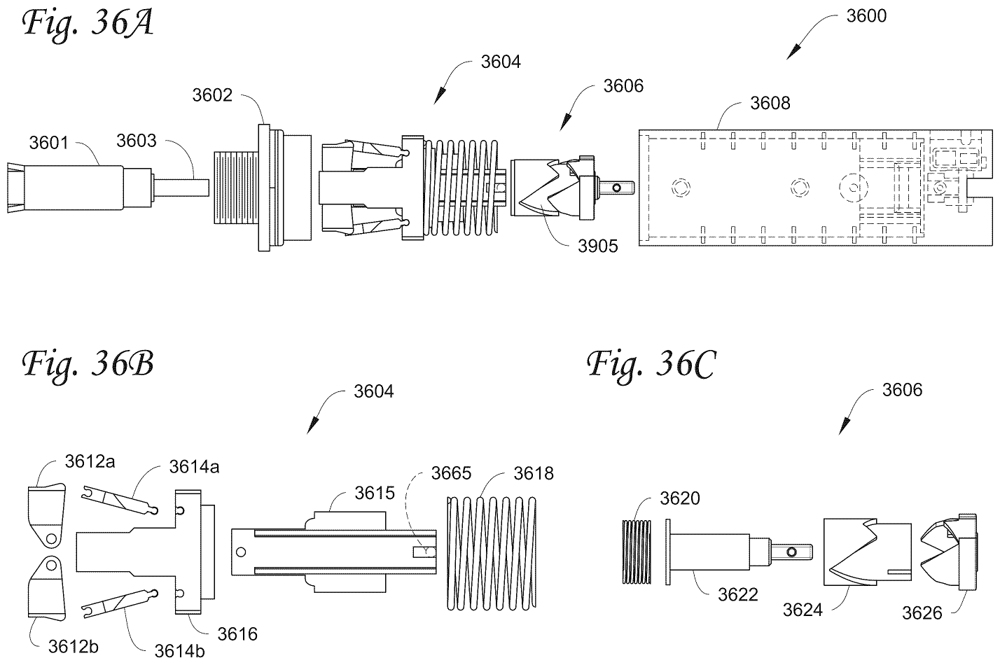

FIGS. 36A-C illustrate side explosion views of portions of a universal pen cap 3600 ready to receive a variety of different dosing pens (e.g., the different dosing pens shown in FIG. 2) having various sizes including the dosing pen 3601 according to another embodiment of the present disclosure. As shown in FIG. 36A, the universal pen cap 3600 includes an adjustable collar 3602, a clamping mechanism 3604, a clicker mechanism 3606 and an optional inner tube 3608. The optional inner tube 3608 can be configured to house at least the clamping mechanism 3604 and the clicker mechanism 3606.

The adjustable collar 3602 is configured to receive a variety of different dosing pens including the dosing pen 3601. The dosing pen 3601 includes a needle and/or needle cover 3603. In some embodiments, the adjustable collar 3602 can be similar to the collar 1502 shown in FIG. 15.

As shown in FIG. 36B, the clamping mechanism 3604 includes two pen clamps 3612a,b, two clamping links 3614a,b, a spring well 3616, a pen datum 3615, and a clamp spring 3618 attached at one end to the spring well 3616. The clamping links 3614a,b along with the clamp spring 3618 can operate similarly to the actuating arms 304 shown in FIGS. 3A-3B to encompass, engage and disengage the dosing or injection pen 3601. In some embodiments, use of a single clamp spring 3618 as opposed to biasing elements 310 on each of the actuating arms 304 can reduce the overall form factor and size of the clamping mechanism 3604.

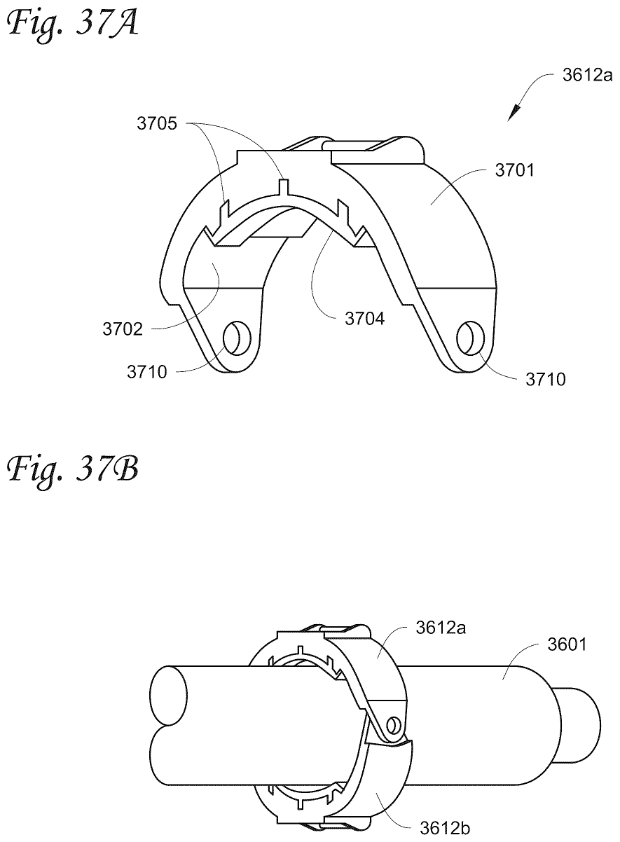

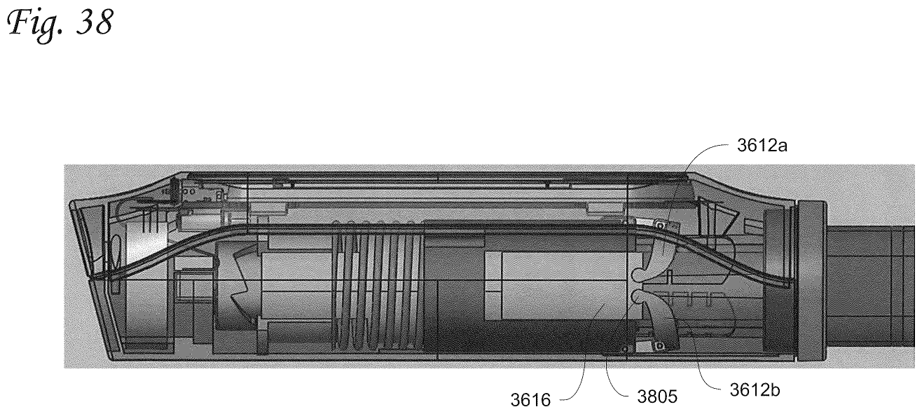

In various embodiments, the pen clamps 3612a,b are attached to the spring well 3616 via a hinge connection. Accordingly, the pen clamps 3612a,b are configured to swing radially inward toward a center longitudinal axis of the universal pen cap 3600 and engage (e.g., contact) the dosing pen 3601 as the dosing pen 3601 is inserted into the universal pen cap 3600. In response to the pen clamps 3612a,b swinging radially inward, the clamping links 3614a,b push the spring well 3616 along the longitudinal axis and compress the clamp spring 3618. As shown in FIGS. 37A-B, each of the pen clamps 3612a,b include a rigid body 3701 and an elastic member 3704 attached to a bottom surface 3702 of the rigid body 3701. The rigid body 3701 can be formed of, for example, a rigid plastic. The elastic member 3704 can be formed of an elastomer material. The elastic member 3704 is configured to allow a variety of dosing pens (including the dosing pen 3601) of varying diameters to have a secure fit within the pen clamps 3612a,b. In some embodiments, the pen clamps 3612a,b may be formed using a two-shot injection molding process. The two-shot injection molding process can create undercuts 3705 that allow for an increased contact surface area between the rigid body 3701 and the elastic member 3704 and thereby increase a mechanical bond strength between the rigid body 3701 and the elastic member 3704. Opposite ends of each of the pen clamps 3612a,b include openings 3710 to accommodate pins (not shown) that can be used for attachment of the pen clamps 3612a,b to the spring well 3616. In another embodiment shown in FIG. 38, opposite ends of the pen clamps 3612a,b can include a ball 3805 that is configured to attach to a socket (not shown) formed in the spring well 3616 using a ball and socket method. By using a ball and socket method to attach the pen clamps 3612a,b (via the ball protrusion 3805) to the spring well 3616 as opposed to pins via openings 3710, as shown in FIGS. 37A-B, the reliability of the pen clamps 3612a,b can be improved for its life cycles of dosing pen insertions and removals.

Returning to FIG. 36B, in various embodiments, the clamping links 3614a,b are coupled to the pen clamps 3612a,b at a longitudinal end of the each of the clamping links 3614a,b. For example, the clamping links 3614a,b in various embodiments can be coupled to the pen clamps 3612a,b via a hinge connection. In various embodiments, the clamp spring 3618 is oriented along a longitudinal axis of the universal pen cap 3600 and is configured to compress responsive to radial movement of the pen clamps 3612a,b.

In some embodiments, at least a portion of the pen datum 3615 is encompassed within the clamp spring 3618. The pen datum 3615 is configured to move along the longitudinal axis of the universal pen cap 3600 within the clamp spring 3618 in engagement with the clicker mechanism 3606. Longitudinal movement of the pen datum 3615 occurs upon insertion of the dosing pen 3601 through the adjustable collar 3602 and the spring well 3616 and into and through the pen datum 3615. In some embodiments, the pen datum 3615 is shaped to allow at least a portion of the needle and/or needle cover 3603 to pass there through. In some embodiments, the pen datum 3615 can include a dead stop feature (not shown) configured to limit the amount of the dosing pen 3601 that is capable of entering into the pen datum 3615. The pen datum 3615 includes two longitudinally extending arms 3665 that form an opening that allows components of the clicker mechanism 3606 to pass therethrough. The pen datum 3615 includes a pen datum protrusion 3670 that protrudes radially inward of the pen datum 3615 and positioned along the longitudinal length of the pen datum 3615 that is configured to ride along a guide path 3905 of the clicker mechanism 3606. In some embodiments, each of the longitudinally extending arms 3665 of the pen datum 3615 includes a pen datum protrusion 3670 that protrudes radially inward of the pen datum 3615. Each of the pend datum protrusions 3670 is configured to ride along the guide path 3905.

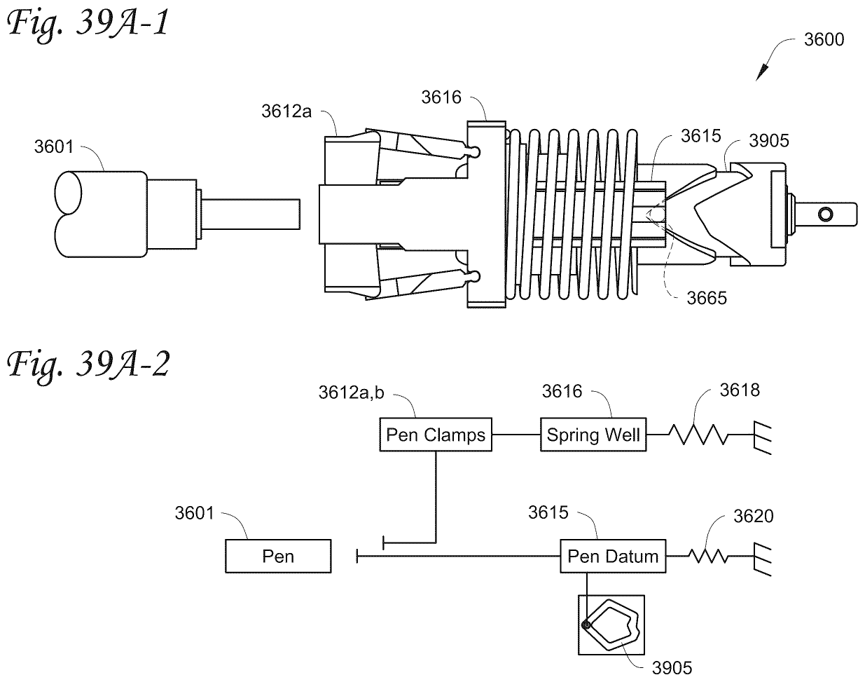

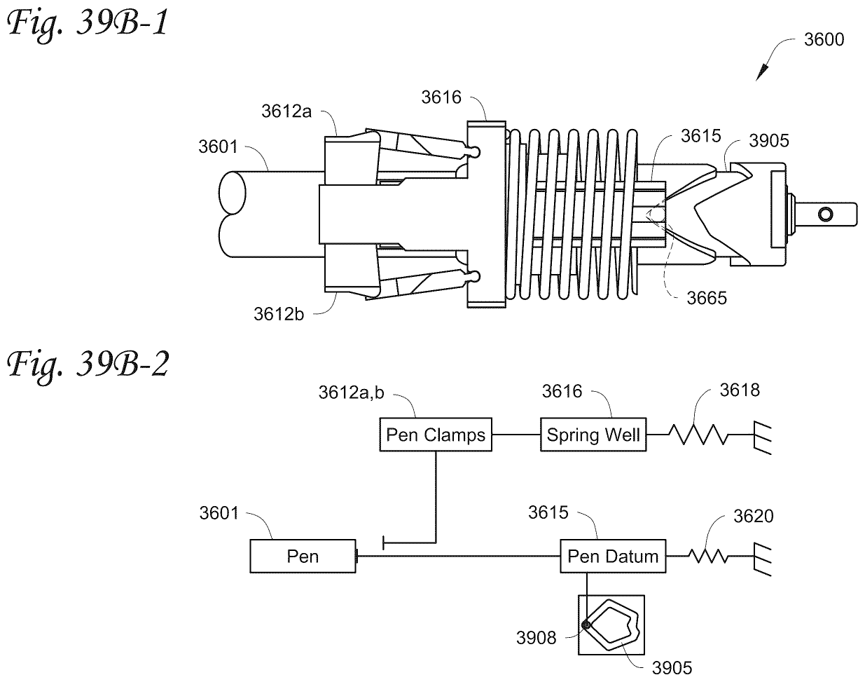

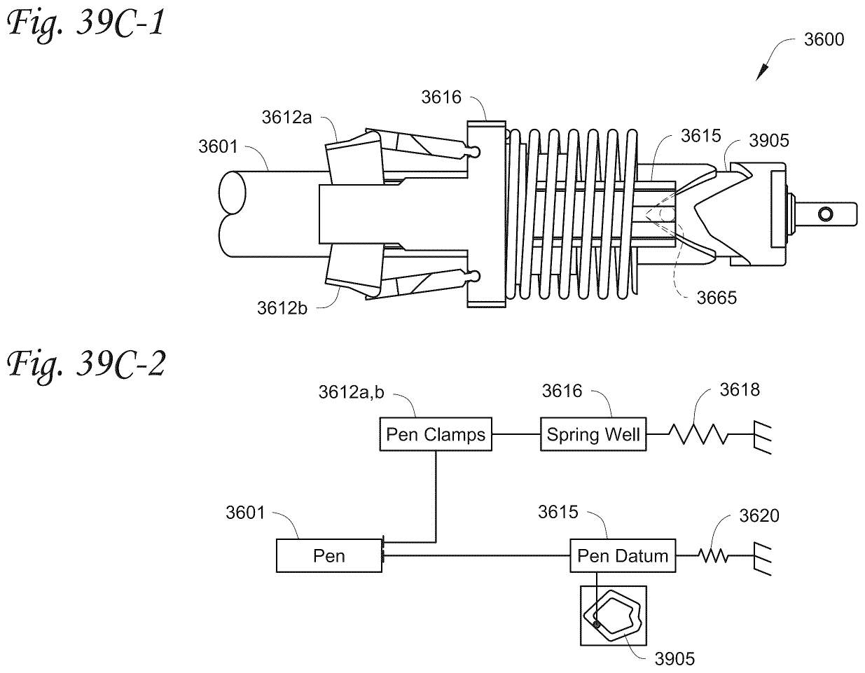

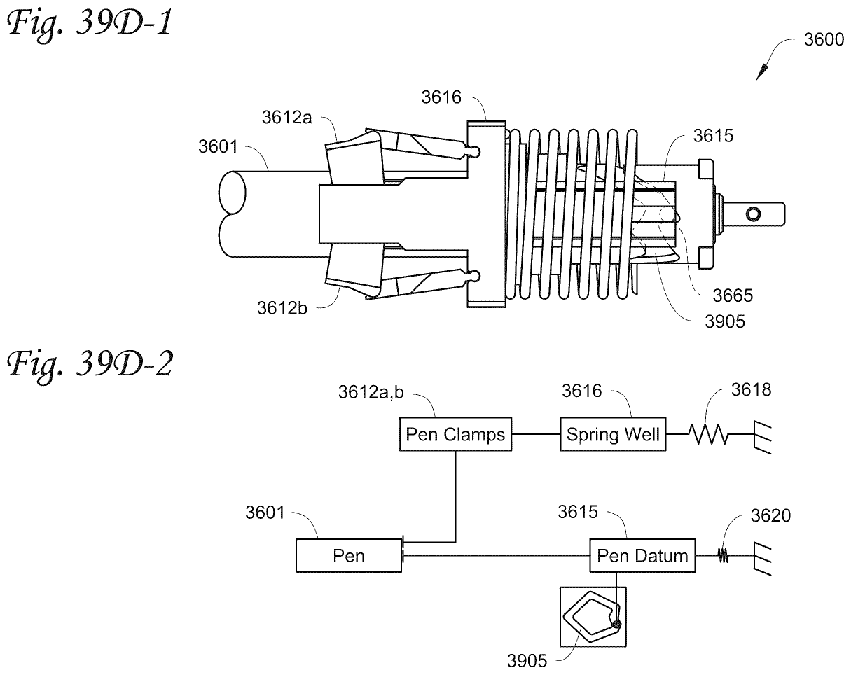

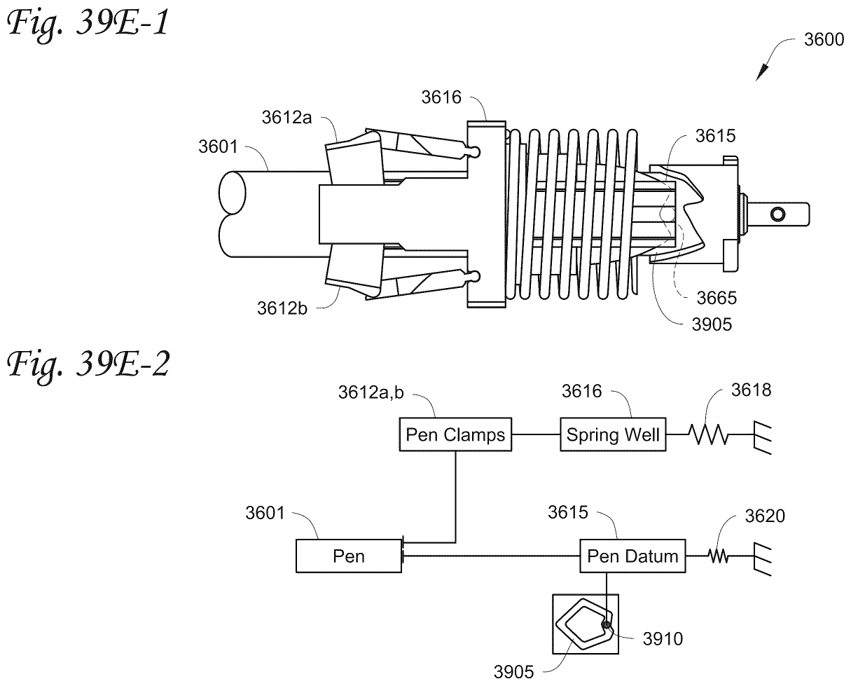

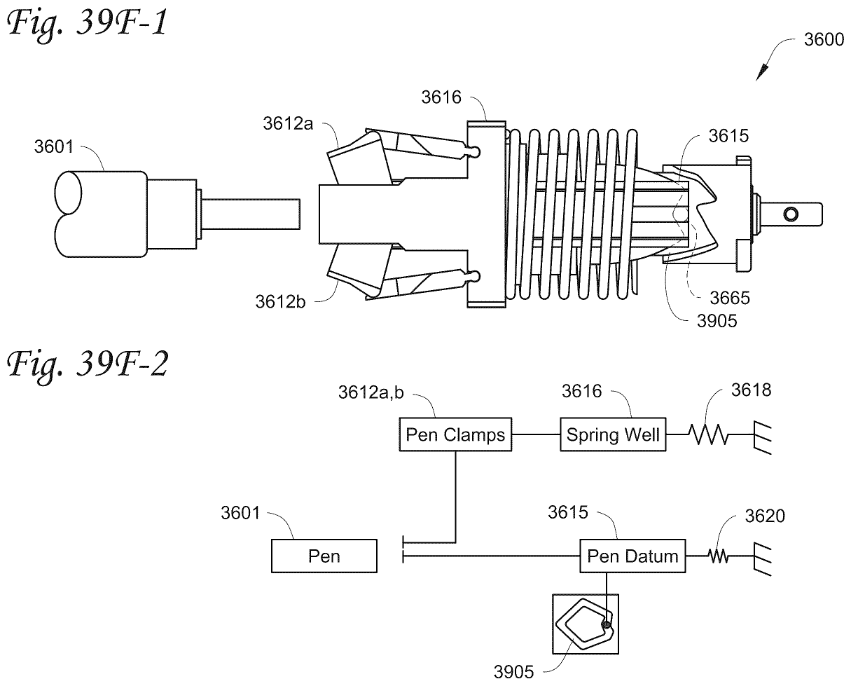

As shown in FIG. 36C, the clicker mechanism 3606 includes a bias member 3620 (e.g., a spring, without limitation), a clicker retainer 3622, and a clicker portion made up of a first clicker body 3624 and a second clicker body 3626. In some embodiments, at least a portion of the needle and/or needle cover 3603 of the dosing pen 3601 is configured to pass through the pen datum 3615 and into the clicker retainer 3622. The pen datum 3615 has an opening along the longitudinal axis that is configured to allow the bias member 3620, the clicker retainer 3622 and at least the first clicker body 3624 to pass therethrough. The clicker retainer 3622 is configured to support the first and second clicker bodies 3624, 3626 thereon. The first and second clicker bodies 3624, 3626 are configured to form the guide path 3905 that wraps around the clicker bodies 3624, 3626. In some embodiments, the guide path 3905 is shaped as a cardioid with zig-zags or ramps wrapping around the clicker bodies 3624, 3626. By using a cardioid shaped guide path 3905 with the clicker mechanism 3606, the form factor and size of the clicker mechanism 3606 can be reduced in comparison, for example, to the cam element 402 and the guide element 412 shown in FIGS. 5A-C. It will be appreciated that the first and second clicker bodies 3624, 3626 are configured to rotate in the same direction during operation of the clicker mechanism 3606. The pen datum 3615 is configured to move forward and backward along a longitudinal axis of the universal pen cap 3600 over the bias member 3620, the clicker retainer 3622 and the first clicker body 3624. The linear travel of the pen datum 3615 along the longitudinal axis of the universal pen cap 3600 causes the inwardly protruding pen datum protrusion(s) 3670 to travel along the guide path 3905 formed by the first and second clicker bodies 3624, 3626 and push the first and second clicker bodies 3624, 3626 to rotate the first and second clicker bodies 3624, 3626 and to compress the bias member 3620. As shown in FIG. 36A, the guide path 3905 can include zig-zags or ramps that the pen datum protrusion(s) 3670 is configured to travel along. Operation of the clicker mechanism 3606 is discussed in more detail below with respect to FIGS. 39A-E.

FIGS. 39A-F illustrate operation of the clicker mechanism 3606 in various operating states as the dosing pen 3601 is inserted into the universal pen cap 3600. FIG. 39A-1 illustrates a portion of the universal pen cap 3600 when the dosing pen 3601 is not in contact with the pen datum 3615 or another part of the clamping mechanism 3604 or the clicker mechanism 3606 (State 0). The pen datum protrusion(s) 3670 rest at a start position 3908 of the guide path 3905. As shown in FIG. 39A-2, both the clamp spring 3618 and the bias member 3620 are in their steady state positions.

FIG. 39B-1 illustrates a portion of the universal pen cap 3600 when the dosing pen 3601 begins to contact the pen datum 3615 but the insertion of the dosing pen 3601 has not started to compress both the clamp spring 3618 and the bias member 3620 (State 1). The pen datum protrusion(s) 3670 continue to rest at the start position 3908 of the guide path 3905. As shown in FIG. 39B-2, both the clamp spring 3618 and the bias member 3620 remain in their steady state positions.

FIG. 39C-1 illustrates a portion of the universal pen cap 3600 when insertion of the dosing pen 3601 has advanced the pen datum 3615 relative to the spring well 3616 (State 2). The clamping links 3614a,b are configured to rotate about the pen datum 3615 and begin to contact an outer perimeter of the dosing pen 3601. As shown in FIG. 39C-2, the clamp spring and optionally the bias member 3620 is configured to compress an amount dependent on the diameter of the dosing pen 3601 being inserted into the universal pen cap 3600. Advancement of the pen datum 3615 has caused the bias member 3620 to start to compress. Also, the pen datum protrusion(s) 3670 have advanced along the ramps 3675 of the guide path 3905 as the first and second clicker bodies 3624, 3626 rotate based on compression of the bias member 3620.

FIG. 39D-1 illustrates a portion of the universal pen cap 3600 when the dosing pen 3601 is fully inserted and further travel is limited by the clicker mechanism 3606 (State 2.5). This position defines the maximum user push force required to latch the dosing pen 3601 into the universal pen cap 3600. As shown in FIG. 39D-2, the pen datum protrusion(s) 3670 have further advanced along the guide path 3905 and caused (along with the dosing pen 3601) compression of both the bias member 3620 and the clamp spring 3618. Advancement of the pen datum 3615 and thus the pen datum protrusion(s) 3670 also causes the first and second clicker bodies 3624, 3626 to rotate. In some embodiments, the radial flexures 4005 on the second clicker body 3626 may be configured to provide haptic and auditory feedback to a user by being forced radially inward by the teeth 4010 on the first clicker body 3624 and then snap radially outward when State 2.5 is reached. In some embodiments, at State 2.5, the bias member 3620 is at or near its maximum compression.

FIG. 39E-1 illustrates a portion of the universal pen cap 3600 when the user has released the dosing pen 3601 and allows the bias member 3620 to move the pen datum 3615 forward (State 3). State 3 defines a clamping force (and thus a retention force) applied to the dosing pen 3601 by the clamping mechanism 3604 and the clicker mechanism 3606. It will be appreciated that the retention force may also be dependent on a coefficient of friction between the dosing pen 3601 and the pen clamps 3612a,b. The coefficient of friction between the dosing pen 3601 and the pen clamps 3612a,b can vary based on the geometry of the dosing pen 3601. As shown in FIG. 39E-2, the pen datum protrusion(s) 3670 are locked into their final state within the clicker mechanism 3606 at a locked position 3910 of the guide path 3905. At locked position 3910, both the bias member 3620 and the clamp spring 3618 are compressed, however the bias member 3620 is not as compressed as it was at State 2.5. Once at State 3 (where the pen datum protrusion(s) 3670 are locked into their final state), the user can remove the dosing pen 3601 from the universal pen cap 3600 by pushing the dosing pen 3601 into the universal pen cap 3600. Pushing the dosing pen 3601 into the universal pen cap 3600 will release the pen datum protrusion(s) 3670 from the locked position 3910 and release the clamp spring 3618 and the bias member 3620 to return the pen datum protrusion(s) 3670 back to the start position 3908 of the guide path 3905.

FIG. 39F-1 illustrates a portion of the universal pen cap 3600 upon a safety removal action of the dosing pen 3601 from the universal pen cap 3600 (State 4). State 4 is defined as the pen datum protrusion(s) 3670 are being locked into their final state within the clicker mechanism 3906, but the dosing pen 3601 is no longer in the universal pen cap 3600. This can occur when the user removes the dosing pen 3601 from the universal pen cap 3600 by pulling the dosing pen 3601 out as opposed to pushing the dosing pen 3601 into the universal pen cap 3600 after the pen datum protrusion(s) 3670 are locked into their final state. As shown in FIG. 39F-2, the pen datum protrusion(s) 3670 remain locked into their final state within the clicker mechanism 3606 at the locked position 3910 of the guide path 3905 and the clamp spring 3618 and the bias member 3620 remain compressed even though the dosing pen 3601 has been removed. In some embodiments, the user can reset the clicking mechanism 3606 back to State 0 by pushing a dosing pen into the universal pen cap 3600 where it will contact the now closed pen clamps 3612a,b. The user continues to insert the dosing pen until the pen datum protrusion(s) 3670 reach a position of the guide path 3905 associated with State 2.5. The user can then release the dosing pen and the clicking mechanism will reset back to State 0.

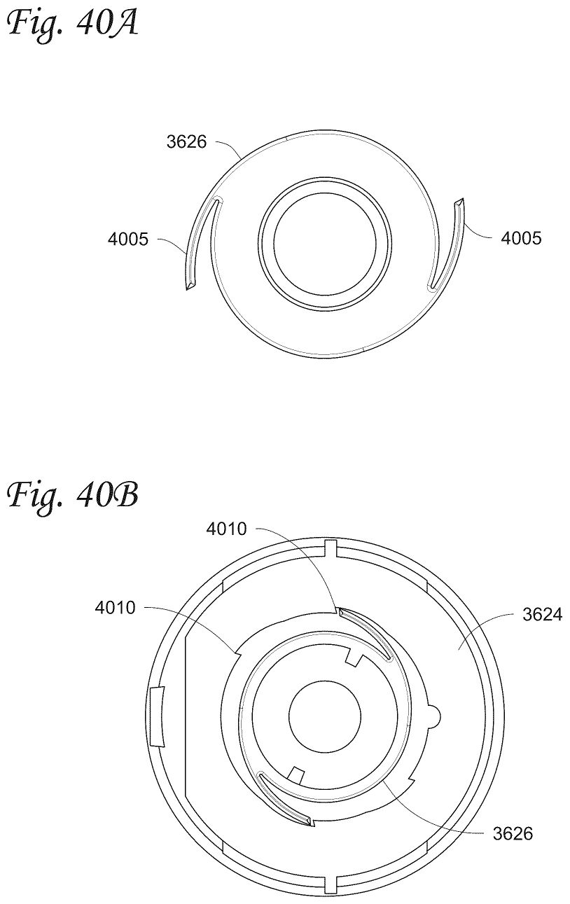

As shown in FIGS. 40A-B, the second clicker body 3626 can include radial haptic feedback flexures 4005 (see FIG. 40A) and the first clicker body 3624 can include teeth 4010 to provide haptic and auditory feedback to a user while inserting the dosing pen 3601 into the universal pen cap 3600. For example, movement of the pen datum 3615 can cause the radial haptic feedback flexures 4005 to be forced radially inward by the teeth 4010 and then snap radially outward upon the clicker mechanism 3606 reaching a designated state. In some embodiments, the pen datum 3615 can cause the radial haptic feedback flexures 4005 to be forced radially inward by the teeth 4010 and then snap radially outward upon the clicker mechanism 3606 reaching State 2.5 as shown in FIGS. 39D-1,2.

In some embodiments, the overall size (e.g., radial size) of the universal pen cap 3600 can be reduced by: decreasing a wall thickness of a housing of the universal pen cap 3600 that houses the clamping mechanism 3604 and the clicking mechanism 3606; and/or removing the optional inner tube 3608 (which used to provide ingress protection).

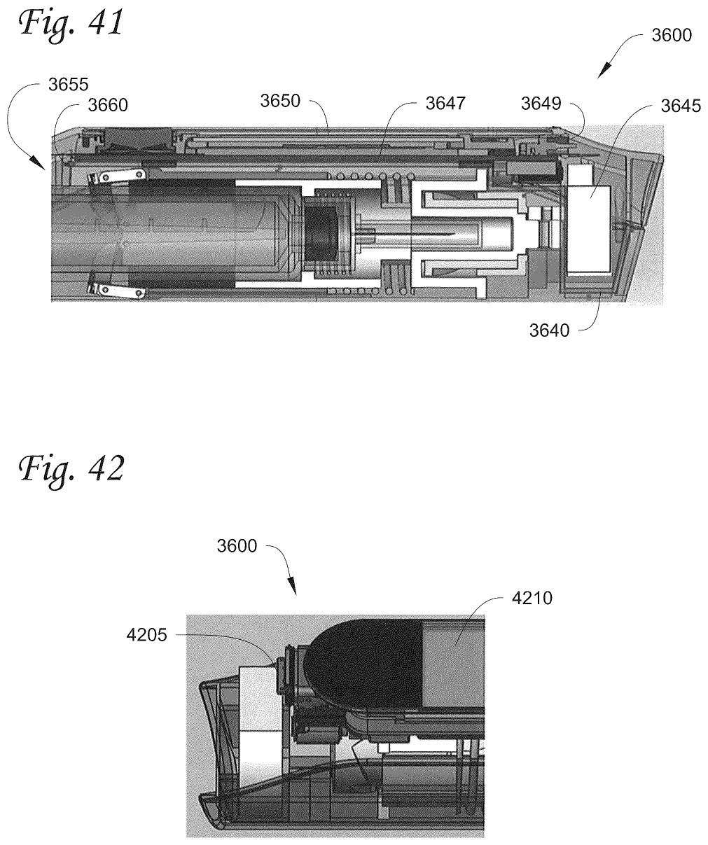

In some embodiments, as shown in FIG. 41, the universal pen cap 3600 can include an ingress wall 3640 protecting electrical components (e.g., a battery 3645, a printed circuit board 3647, a charging port 3649, a display 3650, etc.) of the universal pen cap 3600 from foreign substances entering at or near an open end or mouth 3655 of the universal pen cap 3600, particularly at a location 3660 between the printed circuit board 3647 and the open end 3655. The ingress wall 3640 can run along the longitudinal length of the universal pen cap 3600 and surround, for example, a portion of a battery 3645 used for powering the universal pen cap 3600. In some embodiments the ingress wall 3650 can be attached to an upper housing of the universal pen cap 3600.

In some embodiments, as shown in FIG. 42, the universal pen cap 3600 can include a charging port 4205 positioned adjacent to a pen cap display 4210. By placing the charging port 4205 adjacent to the pen cap display 4210, the charging port 4205 can be protected by the ingress wall 3650 (see FIG. 41).

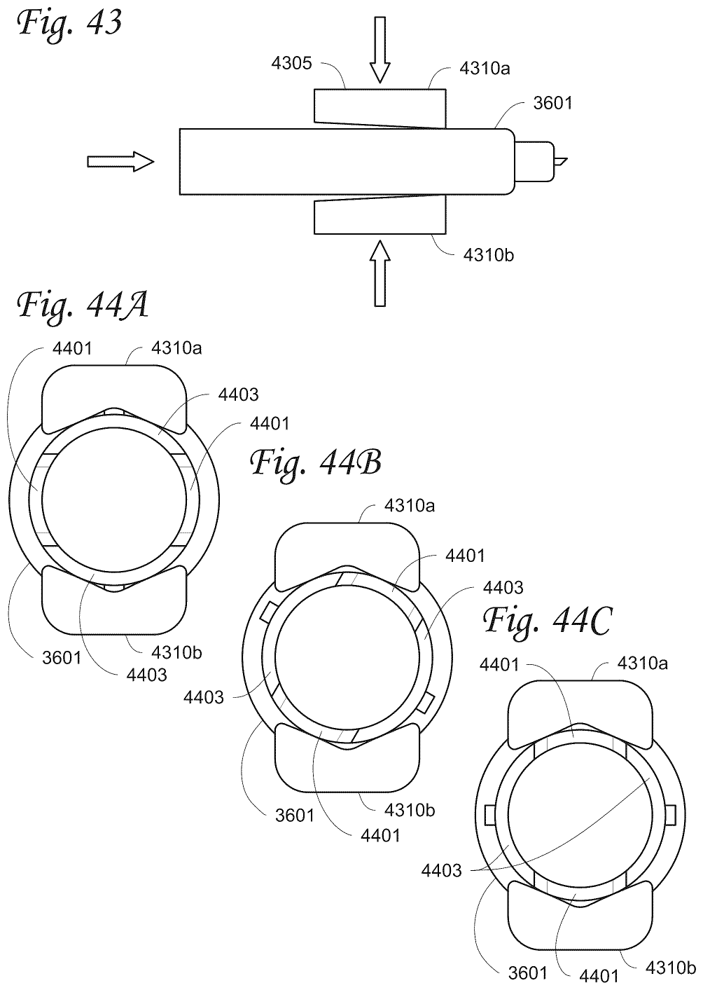

FIG. 43 illustrates a frictional clasp 4305 that can be used in place of the pen clamps 3612a,b shown in FIGS. 36 and 37. The frictional clasp 4305 is configured to radially clamp a dosing pen (e.g., the dosing pen 3601) by two frictional shoes 4310a, b. In some embodiments, the frictional shoes 430 la,b can be spring biased contacts. In this configuration a user directly opposes the frictional force provided by the frictional shoes 4310a,b on both insertion and removal of the dosing pen 3601. Accordingly, the frictional shoes 4310a,b are configured to apply radial force to retain the dosing pen 3601 within a universal pen cap (e.g., the universal pen cap 3600). The frictional shoes 4310a,b can be configured to come into contact with various points of the dosing pen depending on the geometry of the dosing pen. The frictional shoes 4310a,b are tapered at an angle such that the frictional clasp 4305 can always be in contact tangent to the dosing pen irrespective of the rotational orientation of the dosing pen. As shown in FIGS. 44A-C, frictional force of the frictional clasp 4305 on the dosing pen 3601 may vary based on the orientation of the dosing pen 3601. For example, the dosing pen 3601 has a rigid body 4403 and display or cutout portions 4401 that can provide, for example, a view of a dosing substance (e.g., insulin) housed within the dosing pen 3601. However, these display or cutout portions 4401 can provide a reduced frictional contact with the frictional shoes 4301a,b. For example, FIG. 44A shows that each of the frictional shoes 4310a,b form two contact points on the rigid body 4403 of the dosing pen 3601 without the frictional shoes 4310a,b spanning the display or cutout portions 4401. The display or cutout portions 4401 do not span across the frictional shoes 4310a,b. FIG. 44B shows that each of the frictional shoes 4310a,b form one contact point on the rigid body 4403 with the other contact point on the display or cutout portions 4401. FIG. 44C shows that the display or cutout portions 4401 span each of the frictional shoes 4310a,b, while still allowing the frictional shoes 4310a,b to form two contact points on the rigid body 4403.

As used herein, the singular forms following “a,” “an,” and “the” are intended to include the plural forms as well, unless the context clearly indicates otherwise.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

As used herein, any relational term, such as “first,” “second,” etc., is used for clarity and convenience in understanding the disclosure and accompanying drawings, and does not connote or depend on any specific preference or order, except where the context clearly indicates otherwise.

As used herein, the term “substantially” in reference to a given parameter, property, act, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measure of the given parameter, as well as variations resulting from manufacturing tolerances, etc.).

The embodiments of the disclosure described above and illustrated in the accompanying drawing figures do not limit the scope of the invention, since these embodiments are merely examples of embodiments of the invention, which is defined by the appended claims and their legal equivalents. Any equivalent embodiments are intended to be within the scope of this invention. Indeed, various modifications of the present disclosure, in addition to those shown and described herein, such as alternative useful combinations of the content features described, may become apparent to those skilled in the art from the description. Such modifications and embodiments are also intended to fall within the scope of the appended claims and legal equivalents.

Additional non-limiting embodiments of the disclosure include:

Embodiment 1: A pen cap for use with injection pens, the pen cap comprising: one or more adaptable elements configured to removably couple the pen cap to a plurality of different geometries of a plurality of different injection pens.

Embodiment 2: The pen cap according to Embodiment 1, wherein the plurality of different injection pens comprises at least two different injection pens with different geometries.

Embodiment 3: The pen cap according to Embodiments 1 and 2, wherein the one or more adaptable elements comprise: at least one actuator; and a plurality of arms operably coupled to the at least one actuator, wherein the plurality of arms are configured to rotate at least a portion thereof radially inward toward a center longitudinal axis of the pen cap responsive to actuation of the at least one actuator.

Embodiment 4: The pen cap according to Embodiments 1 to 3, wherein the one or more adaptable elements comprise a tube having a plurality of consecutive segments, wherein an inner diameter of the tube incrementally decreases along a center longitudinal axis of the pen cap with each segment of the plurality of consecutive segments including a respective inner diameter.

Embodiment 5: The pen cap according to Embodiments 1 to 4, further comprising an outer frame element including an outer wall, wherein the one or more adaptable elements comprise: a first aperture formed in the outer wall; at least one catch member coupled to the outer frame element and configured to extend through the first aperture and engage an injection pen when an injection pen is inserted into the second aperture of the outer frame element; and a biasing member configured to push the at least one catch member radially inward toward a center longitudinal axis of the pen cap.

Embodiment 6: The pen cap according to Embodiments 1 to 5, wherein the one or more adaptable elements comprise an outer wall including a frusto-conical cavity including a frusto-conical shape formed therein, the frusto-conical cavity including a narrowing inner diameter along a center longitudinal axis of the pen cap with a larger diameter end of the frusto -conical shape at an open end of the outer wall.

Embodiment 7: The pen cap according to Embodiments 1 to 6, further comprising: an outer shell including an outer wall shaped to at least partially surround a longitudinal end of an injection pen, the outer wall including an open end formed therein; a slot formed in the outer wall extending radially through the outer wall and axially along a portion of the outer wall; and wherein the one or more adaptable elements comprise a sleeve element configured to be removably coupled to the longitudinal end of an injection pen of the plurality of injection pens, the sleeve element comprising a radially extending member sized and shaped to slide along the slot when the injection pen is inserted into the pen cap and is adapted to interface with the slot to removably secure the injection pen to the outer shell.

Embodiment 8: The pen cap according to Embodiments 1 to 7, wherein the one or more adaptable elements comprise: a collet comprising a plurality of elongated members oriented circumferentially around a center longitudinal axis of the pen cap and extending in an axial direction relative to the center longitudinal axis of the pen cap, each of the elongated members including a tapered width along a longitudinal length thereof; and an outer sleeve disposed about the plurality of elongated members, the outer sleeve and the plurality of elongated members being configured for relative movement therebetween with the outer sleeve sliding relative to the radially outermost surfaces of the plurality of elongated members based on the relative movement therebetween.

Embodiment 9: The pen cap according to Embodiments 1 to 8, wherein the one or more adaptable elements comprises: a collar including a radially inner surface; a plurality of sloped protrusions extending radially inward from the radially inner surface, a thickness of each of the plurality of sloped protrusions increasing in a circumferential direction, and the plurality of sloped protrusions being evenly spaced circumferentially around the radially inner surface of the collar; and a plurality of roller elements, each roller element being disposed adjacent to a respective sloped protrusion of the plurality of sloped protrusions, the plurality of roller elements configured to contact an outer radial surface of the injection pen, wherein the plurality of roller elements and the plurality of sloped protrusions are adapted for relative movement therebetween in the circumferential direction.

Embodiment 10: The pen cap according to Embodiments 1 to 9, wherein the one or more adaptable elements comprise: a tapered collet, the tapered collet comprising a plurality of recesses formed within radially outer surfaces of the tapered collet, the plurality of recesses oriented relative to one another in a helical pattern, the tapered collet defining a frusto -conical aperture configured to receive at least a portion of an injection pen of the plurality of different injection pens; an annular collar comprising a radially inner surface defining a central axial aperture, the annular collar configured to receive the tapered collet though the central axial aperture and comprising one or more protrusions extending radially inward from the radially inner surface, the one or more protrusions sized, shaped, and positioned to be received into respective recesses of the plurality of recesses and adapted to slide along the respective recesses during operation, wherein translation of the annular collar in a first axial direction along the tapered collet causes at least a portion of the tapered collet to flex radially inward toward a longitudinal axis of the pen cap; and an outer frame member mounted to a radially outer surface of the annular collar.

Embodiment 11: The pen cap according to Embodiments 1 to 10, wherein the one or more adaptable elements comprises: an annular drive gear, the annular drive gear defining a central aperture for receiving at least a portion of an injection pen of the plurality of different injection pens and at least a portion of the annular drive gear is configured to rotate circumferentially about a longitudinal axis of the pen cap; an annular receiving gear operably engaged with the annular drive gear and configured to rotate about an axis thereof, the annular receiving gear including a central aperture formed therein; and an engagement member disposed within the central aperture, wherein the annular receiving gear is adapted to rotate to cause the engagement member to translate along the axis thereof.

Embodiment 12: A pen cap for interfacing with injection pens, the pen cap comprising: means for removably coupling the pen cap to a plurality of different geometric shapes of a plurality of different injection pens.

Embodiment 13: The pen cap according to Embodiment 12, wherein the plurality of different injection pens comprise at least two different injection pens with different geometries.

Embodiment 14: The pen cap according to Embodiments 12 and 13, wherein the means comprises: at least one actuator; and a plurality of arms operably coupled to the at least one actuator, wherein the plurality of arms are configured to rotate at least a portion thereof radially inward toward a center longitudinal axis of the pen cap responsive to actuation of the at least one actuator.

Embodiment 15: The pen cap according to Embodiments 12 to 14, wherein the means comprises a tube having a plurality of consecutive segments, wherein an inner diameter of the tube incrementally decreases along a center longitudinal axis of the pen cap with each segment of the plurality of consecutive segments including a respective inner diameter.

Embodiment 16: The pen cap according to Embodiments 12 to 15, further comprising an outer frame element including an outer wall, wherein the means comprises: an aperture formed in the outer wall; at least one catch member coupled to the outer frame element and configured to extend through the aperture and engage an injection pen when an injection pen is inserted into the aperture of the outer frame element; and a biasing member configured to push the at least one catch member radially inward toward a center longitudinal axis of the pen cap.

Embodiment 17: The pen cap according to Embodiments 12 to 16, wherein the means comprises an outer wall including a frusto-conical cavity including a frusto-conical shape formed therein, the frusto-conical cavity including a narrowing inner diameter along a center longitudinal axis of the pen cap with a larger diameter end of the frusto-conical shape at an open end of the outer wall.

Embodiment 18: The pen cap according to Embodiments 12 to 17, further comprising: An outer shell including an outer wall shaped to at least partially surround a longitudinal end of an injection pen, the outer wall including an open end formed therein; A slot formed in the outer wall extending radially through the outer wall and axially along a portion of the outer wall; and wherein the means comprises a sleeve element configured to be removably coupled to the longitudinal end of an injection pen of the plurality of injection pens, the sleeve element comprising a radially extending member sized and shaped to slide along the slot when the injection pen is inserted into the pen cap and is adapted to interface with the slot to removably secure the injection pen to the outer shell.

Embodiment 19: The pen cap according to Embodiments 12 to 18, wherein the means comprises: a collet comprising a plurality of elongated members oriented circumferentially around a center longitudinal axis of the pen cap and extending in an axial direction relative to the center longitudinal axis of the pen cap, each of the elongated members including a tapered width along a longitudinal length thereof; and an out sleeve disposed about the plurality of elongated members, the outer sleeve and the plurality of elongated members being configured for relative movement therebetween with the outer sleeve sliding relative to the radially outermost surfaces of the plurality of elongated members based on the relative movement therebetween.

Embodiment 20: The pen cap according to Embodiments 12 to 19, wherein the means comprises a lock ring comprising: a collar including a radially inner surface; a plurality of sloped protrusions extending radially inward from the radially inner surface, a thickness of each of the plurality of sloped protrusions increasing in a circumferential direction, and the plurality of sloped protrusions being evenly spaced circumferentially around the radially inner surface of the collar; and a plurality of roller elements, each roller element being disposed adjacent to a respective sloped protrusion of the plurality of sloped protrusions, the plurality of roller elements configured to contact an outer radial surface of the injection pen, wherein the plurality of roller elements and the plurality of sloped protrusions are adapted for relative movement therebetween in the circumferential direction.

Embodiment 21: The pen cap according to Embodiments 12 to 20, wherein the means comprises: a tapered collet, the tapered collet comprising a plurality of recesses formed within radially outer surfaces of the tapered collet, the plurality of recesses oriented relative to one another in a helical pattern, the tapered collet defining a frusto-conical aperture configured to receive at least a portion of an injection pen of the plurality of different injection pens; and an annular collar comprising a radially inner surface defining a central axial aperture, the annular collar configured to receive the tapered collet through the central axial aperture and comprising one or more protrusions extending radially inward from the radially inner surface, the one or more protrusions sized, shaped, and positioned to be received into respective recesses of the plurality of recesses and adapted to slide along the respective recesses during operation, wherein translation of the annular collar in a first axial direction along the tapered collet causes at least a portion of the tapered collet to flex radially inward toward a longitudinal axis of the pen cap; and an outer frame member mounted to a radially outer surface of the annular collar.

Embodiment 22: The pen cap according to Embodiments 12 to 21, wherein the means comprises: an annular drive gear, the annular drive gear defining a central aperture for receiving at least a portion of an injection pen of the plurality of different injection pens and at least a portion of the annular drive gear is configured to rotate circumferentially about a longitudinal axis of the pen cap; an annular receiving gear operably engaged with the annular drive gear and configured to rotate about an axis thereof, the annular receiving gear including a central aperture formed therein; and an engagement member disposed within the central aperture, wherein the annular receiving gear is adapted to rotate to cause the engagement member to translate along the axis thereof.

Embodiment 23: A pen cap for use with injection pens, the pen cap comprising: one or more adaptable elements configured to removably couple the pen cap to a plurality of different geometries of a plurality of different injection pens; and an electromechanical actuator coupled to at least one of the one or more adaptable elements and configured to actuate the one or more adaptable elements to adapt the one or more adaptable elements to a given geometry of a given injection pen.

Embodiment 24: The pen cap according to Embodiment 23, wherein the plurality of different injection pens is at least two different injection pens with different geometries.

Embodiment 25: The pen cap according to Embodiments 23 and 24, wherein the electromechanical actuator comprises at least one actuator chosen from a servo actuator and a solenoid actuator.

Embodiment 26: The pen cap according to Embodiments 23 to 25, further comprising an outer frame element including an outer wall, wherein the one or more adaptable elements comprise: an aperture formed in the outer wall; and at least one catch member coupled to the outer frame element and configured to extend through the aperture and be translated radially inward and outward through the aperture, wherein the electromechanical actuator is configured to translate the at least one catch member radially inward to engage an injection pen responsive to the injection pen being inserted into the aperture of the outer frame element.

Embodiment 27: The pen cap according to Embodiments 23 to 26, wherein the one or more adaptable elements comprises: an outer frame element defining an aperture sized and shaped to receive longitudinal ends of the plurality of injection pens; wherein the electromechanical actuator includes a solenoid actuator and wherein the one or more adaptable elements comprises: one or more engagement elements; a compression element at least partially disposed within the solenoid actuator and adapted to cause the one or more engagement elements to translate along a longitudinal axis of the solenoid actuator, the solenoid actuator positioned such that a longitudinal axis of the solenoid actuator is substantially orthogonal to a longitudinal axis of the pen cap; and wherein the compression element is configured to apply an inward radial force to the one or more engagement elements relative to the longitudinal axis of the pen cap responsive to actuation of the solenoid actuator.

Embodiment 28: The pen cap according to Embodiments 23 to 27, wherein the one or more adaptable elements comprises a clamp apparatus, the clamp apparatus comprising: a first arm; and a second arm, rotatably coupled to the first arm, wherein the electromechanical actuator is coupled to at least one arm chosen from the first arm and the second arm and is configured to rotate at least one arm chosen from the second arm relative to the first arm and the first arm relative to the second arm.

Embodiment 29: The pen cap according to Embodiment 28, further comprising a torsion spring configured to keep the clamp apparatus in a disengaged configuration until actuation of the electromechanical actuator.

Embodiment 30: The pen cap according to Embodiments 23 to 29, wherein the one or more adaptable elements comprises: a collet comprising a plurality of elongated members oriented circumferentially around a center longitudinal axis of the pen cap and extending in an axial direction relative to the center longitudinal axis of the pen cap, each of the elongated members including a tapered width along a longitudinal length thereof; an outer sleeve disposed about the plurality of elongated members, the outer sleeve and the plurality of elongated members being configured for relative movement therebetween with the outer sleeve sliding relative to the radially outermost surfaces of the plurality of elongated members based on the relative movement therebetween; and wherein the electromechanical actuator is configured to cause the relative movement between the outer sleeve and the plurality of elongated members.

Embodiment 31: The pen cap according to Embodiments 23 to 30, wherein the electromechanical actuator is configured to draw power only during periods of configuration-changing events of the pen cap.

Embodiment 32: The pen cap according to Embodiment 31, wherein a configuration-changing event comprises a transition from a first configuration of the pen cap not intended to engage an injection pen of the plurality of injection pens to a second configuration intended to engage the injection pen.

Embodiment 33: The pen cap according to Embodiments 23 to 32, further comprising a sensor coupled to the electromechanical actuator and configured to provide data to the electromechanical actuator, wherein the electromechanical actuator is configured to adjust the pen cap from a first configuration to a second configuration responsive to the data received from the sensor.

Embodiment 34: The pen cap according to Embodiment 33, wherein the data is indicative of a sensed insertion of an injection pen of the plurality of injection pens into the pen cap.

Embodiment 35: The pen cap according to Embodiments 33 and 34, wherein the data is indicative of a sensed attempted removal of the pen cap from an injection pen of the plurality of injection pens.

Embodiment 36: The pen cap according to Embodiments 33 to 35, wherein the sensor comprises at least one type of sensor chosen from a motion sensor, proximity sensor, a pressure sensor, and an optical sensor.

Embodiment 37: The pen cap according to Embodiments 23 to 36, further comprising an actuation device coupled to the electromechanical actuator and configured to adjust the pen cap from a first configuration to a second configuration responsive to actuation of the actuation device.

Embodiment 38: The pen cap according to Embodiment 37, wherein the actuation device comprises an input chosen from a button and a switch.

Embodiment 39: A pen cap for interfacing with injection pens, the pen cap comprising: means for removably coupling the pen cap to a plurality of different geometric shapes of a plurality of different injection pens; and an electromechanical actuator operably coupled to at least one element of the means and configured to at least partially effectuate operation of the means.

Embodiment 40: The pen cap according to Embodiment 39, wherein the plurality of different injection pens comprise at least two different injection pens with different geometries.

Embodiment 41: The pen cap according to Embodiments 39 and 40, wherein the electromechanical actuator comprises at least one actuator chosen from a servo actuator and a solenoid actuator.

Embodiment 42: The pen cap according to Embodiments 39 to 41, further comprising an outer frame element including an outer wall, wherein the means comprises: an aperture formed in the outer wall; and at least one catch member coupled to the outer frame element and configured to extend through the aperture and be translated radially inward and outward through the aperture, wherein the electromechanical actuator is configured to translate the at least one catch member radially inward to engage an injection pen responsive to the injection pen being inserted into the aperture of the outer frame element.

Embodiment 43: The pen cap according to Embodiments 39 to 42, wherein the means comprises: an outer frame element defining an aperture sized and shaped to receive longitudinal ends of the plurality of injection pens; wherein the electromechanical actuator includes a solenoid actuator and wherein the means comprises: one or more engagement elements; a compression element at least partially disposed within the solenoid actuator and adapted to cause the one or more engagement elements to translate along a longitudinal axis of the solenoid actuator, the solenoid actuator positioned such that a longitudinal axis of the solenoid actuator is substantially orthogonal to a center longitudinal axis of the pen cap; and wherein the compression element is configured to apply an inward radial force to the one or more engagement elements relative to the center longitudinal axis of the pen cap responsive to actuation of the solenoid actuator.

Embodiment 44: The pen cap according to Embodiments 39 to 43, wherein the means comprises a clamp apparatus, the clamp apparatus comprising: a first arm; and a second arm, rotatably coupled to the first arm, wherein the electromechanical actuator is coupled to at least one arm chosen from the first arm and the second arm and is configured to rotate at least one arm chosen from the second arm relative to the first arm and the first arm relative to the second arm.

Embodiment 45: The clamp apparatus according to Embodiment 44, further comprising a torsion spring configured to keep the clamp apparatus in a disengaged configuration until actuation of the electromechanical actuator.

Embodiment 46: The pen cap according to Embodiments 39 to 45, wherein the means comprises: a collet comprising a plurality of elongated members oriented circumferentially around a center longitudinal axis of the pen cap and extending in an axial direction relative to the center longitudinal axis of the pen cap, each of the elongated members including a tapered width along a longitudinal length thereof; an outer sleeve disposed about the plurality of elongated members, the outer sleeve and the plurality of elongated members being configured or relative movement therebetween with the outer sleeve sliding relative to the radially outermost surfaces of the plurality of elongated members based on the relative movement therebetween; and wherein the electromechanical actuator is configured to cause the relative movement between the outer sleeve and the plurality of elongated members.

Embodiment 47: The pen cap according to Embodiments 39 to 46, wherein the electromechanical actuator is configured to draw power only during periods of configuration-changing events of the pen cap.

Embodiment 48: The pen cap according to Embodiments 39 to 47, further comprising a sensor coupled to the electromechanical actuator and configured to provide data to the electromechanical actuator, wherein the electromechanical actuator is configured to adjust the pen cap from a first configuration to a second configuration responsive to the data received from the sensor.

Embodiment 49: The pen cap according to Embodiment 48, wherein the data is indicative of a sensed insertion of an injection pen of the plurality of injection pens into the pen cap.

Embodiment 50: The pen cap according to Embodiments 48 and 49, wherein the data is indicative of a sensed attempted removal of the pen cap from an injection pen of the plurality of injection pens.

Embodiment 51: The pen cap according to Embodiments 48 to 50, wherein the sensor comprises at least one type of sensor chosen from a motion sensor, proximity sensor, a pressure sensor, and an optical sensor.

Embodiment 52: The pen cap according to Embodiments 39 to 51, further comprising an actuation device coupled to the electromechanical actuator and configured to adjust the pen cap from a first configuration to a second configuration responsive to actuation of the actuation device.

Embodiment 53: The pen cap according to Embodiment 52, wherein the actuation device comprises an input chosen from a button and a switch.

Embodiment 54: A method of actuating an electromechanical pen cap comprising: detecting a pen cap event using one or more sensors; actuating a clasping mechanism responsive to the event.

Embodiment 55: The method according to Embodiment 54, further comprising: detecting an attempted insertion of an injection pen using the one or more sensors; detecting that the clasping mechanism is open; detecting a completed insertion of the injection pen into the pen cap; and actuating the clasping mechanism to a closed position.

Embodiment 56: The method according to Embodiments 54 and 55, further comprising: detecting a force indicative of an attempted removal of an injection pen from the pen cap; detecting that the clasping mechanism is closed; actuating the clasping mechanism to an open position responsive to detecting that the force is greater than a predetermined threshold; and confirming the clasping mechanism is in an open position.

Embodiment 57: A pen cap for interfacing with medical injection pens, the pen cap comprising: one or more adjustable floor elements configured to adjust a distance an injection pen is insertable into the pen cap; wherein the one or more adjustable floor elements comprise a floor block element defining a cavity configured to accommodate a geometry of the medical injection pens.

Embodiment 58: The pen cap according to Embodiment 57, wherein the one or more adjustable floor elements further comprise: an electromechanical actuator; a threaded screw element operably coupled to the electromechanical actuator; an outer tube element configured to accommodate the floor block element; wherein the floor block element comprises a receiving threaded portion configured to receive the threaded screw element.

Embodiment 59: The pen cap according to Embodiments 57 and 58, wherein the one or more adjustable floor elements further comprise: a body including an annular shape with an outer circumferential surface and an inner circumferential surface defining a cavity; a plurality of ridges disposed circumferentially around at least part of the outer circumferential surface; one or more flexible arms disposed within the cavity and extending in an axial direction adjacent to the inner circumferential surface and cantilevered over at least part of the inner circumferential surface.

Embodiment 60: A pen cap for use with injection pens, the pen cap comprising: one or more adaptable elements configured to removably couple the pen cap to a plurality of different geometries of a plurality of different injection pens.

Embodiment 61: The pen cap of embodiment 60, wherein the plurality of different injection pens comprises at least two different injection pens with different geometries.

Embodiment 62: he pen cap of embodiment 60, wherein the one or more adaptable elements includes a clamping mechanism and a clicker mechanism, wherein the clicker mechanism is configured to removably couple any of the plurality of injection pens by mechanically adapting the clamping mechanism and the clicker mechanism to a given injection pen having a given geometry.

Embodiment 63: The pen cap of embodiment 60, wherein the clamping mechanism includes a clamp spring, a spring well accommodating the clamp spring oriented along a longitudinal axis of the pen cap, a pen datum configured to move along the longitudinal axis of the pen cap within a portion of the spring well and the clamp spring, a pen clamp movably attached to the spring well via a hinge connection so as to swing radially inward toward the longitudinal axis of the pen cap, and a clamping link attaching the clamp link to the spring well, wherein the pen clamp is configured to compress the clamp spring, via the clamping link and the spring well, as the injection pen is inserted into the pen cap.

Embodiment 64: The pen cap of embodiment 63, wherein the clicker mechanism includes a bias member oriented along a longitudinal axis of the pen cap, a clicker portion, and a clicker retainer supporting the clicker portion, wherein the clicker portion forms a guide path that wraps around the clicker portion, wherein the wherein the clicker portion is configured to rotate as the pen datum moves along the longitudinal axis of the pen cap.

Embodiment 65: The pen cap of embodiment 64, wherein the guide path is shaped as a cardioid wrapping around the clicker portion.

Embodiment 66: The pen cap of embodiment 64, wherein the clicker portion includes a first clicker body and a second clicker body configured to rotate in the same direction as the pen datum moves along the longitudinal axis of the pen cap.

Embodiment 67: The pen cap of embodiment 66, wherein the first clicker body includes one or more teeth and the second clicker body includes one or more radial flexures, wherein the one or more radial flexures are configured to provide haptic and/or auditory feedback to a user by being forced radially inward by the one or more teeth during rotation of the clicker portion and then snapping radially outward upon the clicker mechanism reaching a desired operation state.

Embodiment 68: The pen cap of embodiment 63, wherein the pen clamp includes a ball protrusion that is configured to movably attach to the spring well via a ball and socket method.

Embodiment 69: The pen cap of embodiment 63, wherein the pen clamp includes a rigid body and an elastic member attached to a bottom surface of the rigid body, wherein the rigid body includes undercuts for the elastic member to fit within to increase a contact surface area between the rigid body and the elastic member.

Embodiment 70: The pen cap of embodiment 63, wherein the pen clamp includes two frictional shoes configured to apply a radial force to the given injection pen being inserted into the pen cap.

Embodiment 71. The pen cap of embodiment 60, further comprising an ingress wall protecting electronic components of the pen cap from foreign substances.

Embodiment 72: The pen cap of embodiment 64, wherein the clicker mechanism is configured to operate in a plurality of operation states as the given injection pen is inserted into the pen cap.

Embodiment 73: The pen cap of embodiment 72, wherein the plurality of operation states includes a locked state in which the pen datum is locked in the clicker mechanism and the given injection pen is inserted into the pen cap.

Embodiment 74: The pen cap of claim 72, wherein the plurality of operation states includes a safety removal action state in which the pen datum remains locked in the clicker mechanism as the given injection pen is forcibly pulled out of the pen cap.

What is claimed is:

1. A pen cap for use with injection pens, the pen cap comprising:
one or more adaptable elements configured to removably couple the pen cap to a plurality of different geometries of a plurality of different injection pens.

2. The pen cap of claim 1, wherein the plurality of different injection pens comprises at least two different injection pens with different geometries.

3. The pen cap of claim 1, wherein the one or more adaptable elements includes a clamping mechanism and a clicker mechanism,
wherein the clicker mechanism is configured to removably couple any of the plurality of injection pens by mechanically adapting the clamping mechanism and the clicker mechanism to a given injection pen having a given geometry.

4. The pen cap of claim 3, wherein the clamping mechanism includes a clamp spring, a spring well accommodating the clamp spring oriented along a longitudinal axis of the pen cap, a pen datum configured to move along the longitudinal axis of the pen cap within a portion of the spring well and the clamp spring, a pen clamp movably attached to the spring well via a hinge connection so as to swing radially inward toward the longitudinal axis of the pen cap, and a clamping link attaching the clamp link to the spring well,
wherein the pen clamp is configured to compress the clamp spring, via the clamping link and the spring well, as the injection pen is inserted into the pen cap.

5. The pen cap of claim 4, wherein the clicker mechanism includes a bias member oriented along a longitudinal axis of the pen cap, a clicker portion, and a clicker retainer supporting the clicker portion, wherein the clicker portion forms a guide path that wraps around the clicker portion, wherein the wherein the clicker portion is configured to rotate as the pen datum moves along the longitudinal axis of the pen cap.

6. The pen cap of claim 5, wherein the guide path is shaped as a cardioid wrapping around the clicker portion.

7. The pen cap of claim 5, wherein the clicker portion includes a first clicker body and a second clicker body configured to rotate in the same direction as the pen datum moves along the longitudinal axis of the pen cap.

8. The pen cap of claim 7, wherein the first clicker body includes one or more teeth and the second clicker body includes one or more radial flexures, wherein the one or more radial flexures are configured to provide haptic and/or auditory feedback to a user by being forced radially inward by the one or more teeth during rotation of the clicker portion and then snapping radially outward upon the clicker mechanism reaching a desired operation state.

9. The pen cap of claim 4, wherein the pen clamp includes a ball protrusion that is configured to movably attach to the spring well via a ball and socket method.

10. The pen cap of claim 4, wherein the pen clamp includes a rigid body and an elastic member attached to a bottom surface of the rigid body, wherein the rigid body includes undercuts for the elastic member to fit within to increase a contact surface area between the rigid body and the elastic member.

11. The pen cap of claim 4, wherein the pen clamp includes two frictional shoes configured to apply a radial force to the given injection pen being inserted into the pen cap.

12. The pen cap of claim 1, further comprising an ingress wall protecting electronic components of the pen cap from foreign substances.

13. The pen cap of claim 5, wherein the clicker mechanism is configured to operate in a plurality of operation states as the given injection pen is inserted into the pen cap.

14. The pen cap of claim 13, wherein the plurality of operation states includes a locked state in which the pen datum is locked in the clicker mechanism and the given injection pen is inserted into the pen cap.

15. The pen cap of claim 13, wherein the plurality of operation states includes a safety removal action state in which the pen datum remains locked in the clicker mechanism as the given injection pen is forcibly pulled out of the pen cap.
